(19) United States
(12) Patent Application Publication
(10) Pub. No.: US 2024/0075100 A1
(43) Pub. Date: Mar. 7, 2024

(54) Compositions Containing Protein Loaded Exosome and Methods for Preparing and Delivering the Same

(71) Applicant: ILIAS BIOLOGICS INC., Daejeon (KR)

(72) Inventors: Chulhee Choi, Daejeon (KR); Nambin Yim, Daejeon (KR); Won Do Heo, Daejeon (KR); Seung-Wook Ryu, Daejeon (KR); Hojun Choi, Daejeon (KR); Kyungsun Choi, Daejeon (KR)

(73) Assignee: ILIAS BIOLOGICS INC., Daejeon (KR)

(21) Appl. No.: 18/506,575

(22) Filed: Nov. 10, 2023

Related U.S. Application Data
(63) Continuation of application No. 16/887,029, filed on May 29, 2020, now Pat. No. 11,872,193.
Application No. 16/887,029 is a continuation of application No. 15/803,338, filed on Nov. 3, 2017, now Pat. No. 10,702,581.
Application No. 15/803,338 is a continuation-in-part of application No. PCT/KR2017/011070, filed on Sep. 30, 2017.
Application No. PCT/KR2017/011070 is a continuation-in-part of application No. PCT/KR2016/004750, filed on May 4, 2016.

(30) Foreign Application Priority Data
May 4, 2015 (KR) 10-2015-0062604
Aug. 27, 2015 (KR) 10-2015-0120934
Sep. 30, 2016 (KR) 10-2016-0126335
Sep. 30, 2016 (KR) 10-2016-0126921
Sep. 30, 2016 (KR) 10-2016-0126961
Oct. 4, 2016 (KR) 10-2016-0127486
Oct. 13, 2016 (KR) 10-2016-0132616
Feb. 10, 2017 (KR) 10-2017-0018637

(51) Int. Cl. A61K 38/17; A61K 47/69; C07K 14/705; C07K 16/18; C07K 16/28; C12N 15/62; C12N 15/85; C12P 21/02

(52) U.S. Cl. CPC A61K 38/177; A61K 47/6917; C07K 14/705; C07K 14/70596; C07K 16/18; C07K 16/28; C12N 15/62; C12N 15/85; C12P 21/02; A61K 38/00; C07K 2317/80; C07K 2319/03; C07K 2319/70

(57) ABSTRACT

The present invention relates to a method for the mass-production of exosome comprising a cargo protein, a vector for preparing the exosome, exosome including a cargo protein prepared by the method, and a method for loading the cargo protein to cytosol by using the exosome prepared thereby. According to the method for preparing an exosome comprising a cargo protein provided by the present invention, the exosome loaded with a cargo protein can be produced with a high yield, so that it can be used broadly in the treatment of disease using the exosome.

### CROSS-REFERENCE TO RELATED APPLICATIONS

This application is a continuation of U.S. patent application Ser. No. 15/803,338 filed Nov. 3, 2017, which is a continuation-in-part of PCT/KR2016/004750 filed May 4, 2016, and a continuation-in-part of PCT/KR2017/011070, filed Sep. 30, 2017, wherein PCT/KR2016/004750 claims the benefit of priority from Korean Patent Application No. 10-2015-0062604 filed May 4, 2015, Korean Patent Application No. 10-2015-0120934 filed Aug. 27, 2015, and Korean Patent Application No. 10-2016-0055687 filed May 4, 2016, and wherein PCT/KR2017/011070 claims benefit from Korean Patent Application No. 10-2016-0126335 filed Sep. 30, 2016, Korean Patent Application No. 10-2016-0126921 filed Sep. 30, 2016, Korean Patent Application No. 10-2016-0126961 filed Sep. 30, 2016, Korean Patent Application No. 10-2016-0127486 filed Oct. 4, 2016, Korean Patent Application No. 10-2016-0132616 filed Oct. 13, 2016, and Korean Patent Application No. 10-2017-0018637 filed Feb. 10, 2017, the contents of each of which are incorporated herein by reference.

### FIELD OF THE INVENTION

The present invention relates to compositions containing protein loaded exosome, methods for preparing exosome loaded with a cargo protein using a photo-specific binding protein, and a method for delivering the cargo protein to cytosol using the exosome prepared thereby.

### BACKGROUND OF THE INVENTION

The human body is composed of about 200 kinds of 100 trillion cells, in which the physiological activity is regulated by the action of various proteins to maintain life.

Cells are surrounded by membranes in bilayer structure composed of phospholipids, which block the entry of foreign substances into cells. Most of the protein drugs which have developed so far cannot pass through the cell membrane to enter the cell and can act on the outside of the cell or act on a receptor on the cell membrane to deliver the signal into the cell in order to show physiological effect.

Cytosol has lots of proteins which interact with each other to regulate physiological activity. So, if only a protein drug can be delivered inside the cell, that is, inside the cytosol, the cell activity would be controlled more effectively.

Recently, studies have been actively going on to establish a method for delivering a cargo protein directly into cells via cell membrane. When a recombinant protein of a cargo protein and protein transduction domains (PTDs), the peptide that passes through the cell membrane, is prepared and administered, it can enter the cytosol through the cell membrane (FIG. 1). PTD is exemplified by HIV-1 TAT, HSV VP22, Antp, dfTAT, and Hph-1. A fusion protein prepared by combining the PTDs and a cargo protein is produced as a recombinant protein and at this time a separation process is required. However, this process is problematic in that the protein refolding is not performed properly, the activity is decreased, the protein is nonspecifically transferred, the risk of causing an immune reaction in vivo is large, the cost is high, and the yield is low.

The cargo protein conjugated with various nanoparticles can enter the cytosol through the cell membrane by endocytosis (FIG. 2). At this time, the nanoparticles are exemplified by Gold NP, Liposome NP, Magnetic NP, and Polymeric NP, etc. The separation of the nanoparticles from the cargo protein occurs mostly in lysosome in the cell, so the cargo protein is decomposed inside lysosome to lose its activity. Or the nanoparticles are difficult to be separated from the cargo protein in cytosol and toxicity of the nanoparticles can be another problem.

Exosome is a small vesicle with a membrane structure in the size of 50˜200 nm, which is secreted out of the cell with containing protein, DNA, and RNA for intercellular signaling.

Exosome was first found in the process of leaving only hemoglobin in the red blood cells by eliminating intracellular proteins at the last stage of red blood cell maturation. From the observation under electron microscope, it was confirmed that exosome is not separated directly from plasma membrane but discharged extracellular from the intracellular specific zone, called multi-vesicular bodies (MVBs). That is, when MVBs are fused with plasma membrane, such vesicles are discharged outside of the cell, which are called exosome (FIG. 3).

It has not been clearly disclosed the molecular mechanism of the exosome generation. However, it is known that various immune cells including B-lymphocytes, T-lymphocytes, dendritic cells, megakaryocytes, and macrophages, stem cells, and tumor cells produce and secrete exosomes when they are alive.

Exosome contains various intracellular proteins, DNA, and RNA. The substances secreted out of the cells contained in these exosomes can be reintroduced into other cells by fusion or endocytosis and serve as intercellular messengers. By analyzing such substances that are secreted out of the cell as included in exosome, specific disease can be diagnosed.

Exosome also includes various types of microRNAs. A method for diagnosing a disease by detecting the presence or absence and the abundance thereof has been reported (KR 10-2010-0127768A). International Patent Publication No. WO2009-015357A describes a method for predicting and diagnosing a specific disease by detecting exosome in the cancer patient originated samples (blood, saliva, tears, etc.). In particular, the exosome obtained from a patient having a specific disease (lung disease) is analyzed and the relationship between a specific microRNA and lung disease is specifically described. Studies have been still going on to establish a method to diagnose kidney disease, in addition to lung disease, by using a specific protein included in exosome.

Exosome might also include antigens. In antigen presenting cells (APC), antigen peptide is loaded in MHC (major histocompatibility complex) class II molecule in the intracellular compartment having a membrane structure including polycystic bodies. Therefore, the exosome originated therefrom also has the antigen peptide-MHC class II complex. So, exosome acts as an immunogen carrier to present antigen peptide to CD4+ T lymphocytes and thereby can induce immune response such as T lymphocyte proliferation. The molecules that are able to stimulate immune response such as MHC class I and heat-shock proteins (HSPs) are concentrated in exosome, so that exosome can be used to increase or decrease immune response for the treatment of cancer or auto-immune disease.

### SUMMARY OF THE INVENTION

The present invention provides compositions containing exosome loaded with a cargo protein.

In another embodiment, the present invention provides a method for preparing the exosome loaded with a cargo protein using a photo-specific binding protein.

In a further embodiment, the present invention provides a method of delivering the cargo protein to cytosol using the exosome.

### BRIEF DESCRIPTION OF THE DRAWINGS

FIG. 1 illustrates the method for delivering a cargo protein through a recombinant protein of a cargo protein and protein transduction domains (PTDs) (Steven R. et al. Protein transduction: unrestricted delivery into all cells Trends in Cell Biology, 2000).

FIG. 2 illustrates the method for delivering a cargo protein to cytosol using a complex of nanoparticles and a cargo protein via endocytosis (Munish Chanana et al. Physicochemical properties of protein-coated gold nanoparticles in biological fluids and cells before and after proteolytic digestion. Angew. Chem. Int. Ed. 2013).

FIG. 3 illustrates the process in which exosome is separated and released from multi-vesicular bodies (MVBs) (Graca Raposo and Willem Stoorvogel. Extracellular vesicles: Exosomes, microvesicles, and friends. Cell Biology 200(4), 373-383, 2013).

FIG. 4 illustrates the process of treating cancer by delivering siRNA in vivo through the targeted exosome (Alvarez-Erviti, L. et al. Delivery of siRNA to the mouse brain by systemic injection of targeted exosomes. Nature Biotechnology 29, 341-345, 2011).

FIG. 5 illustrates the preparation process of optogenetically-designed protein-carrying exosomes (EXPLORs) according to the present invention.

FIG. 6 illustrates the process of separating the fusion protein of a cargo protein and a photo-specific binding protein in the inside of exosome when the light irradiation on EXPLORs is stopped.

FIG. 7 illustrates the changes in the intracellular location of mCherry protein according to the blue light irradiation in the transformed HEK293T cells introduced with CIBN-EGFP-CD9 gene and mCherry-CRY2 gene.

FIG. 8 illustrates the experimental procedure of obtaining EXPLORs according to the present invention.

FIG. 9 illustrates the results of measuring the changes of the content of a cargo protein (mCherry protein) captured in exosome according to the intensity of blue light.

FIG. 10 illustrates the results of investigation of the introduction of a cargo protein in target cells after treating the target cells (HT1080) with exosome containing the cargo protein (mCherry), wherein the left indicates the target cells not-treated with exosome and the right indicates the target cells treated with exosome.

FIG. 11 is a set of a fluorescence image (a) illustrating the results of investigation of the introduction of a cargo protein in target cells after treating the target cells (HT1080) with exosome containing the cargo protein (mCherry); and a graph (b) illustrating the results of comparison of the ratio of apoptotic cells induced by the treatment of exosome.

FIG. 12 illustrates the changes in the intracellular location of mCherry protein according to the blue light irradiation in the transformed HEK293T cells introduced with GIGANTEA-EGFP-CD9 gene and mCherry-FKF1LOV.

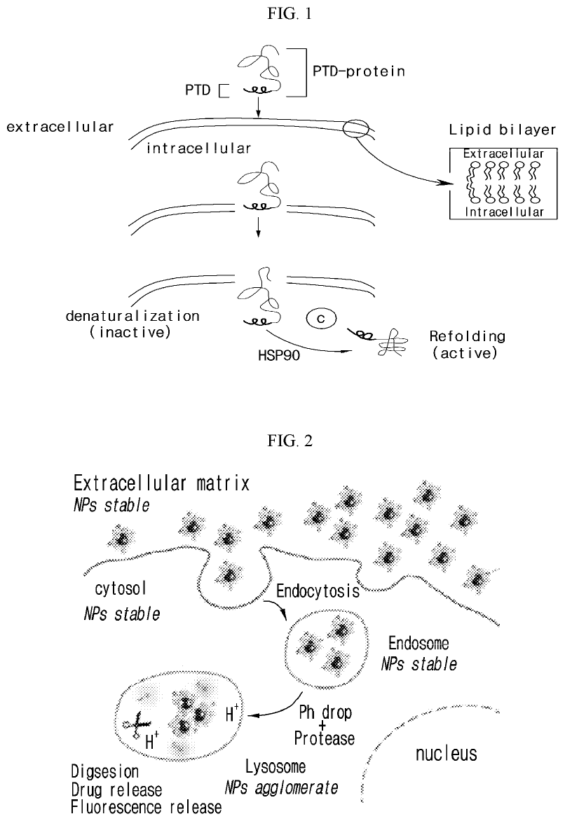

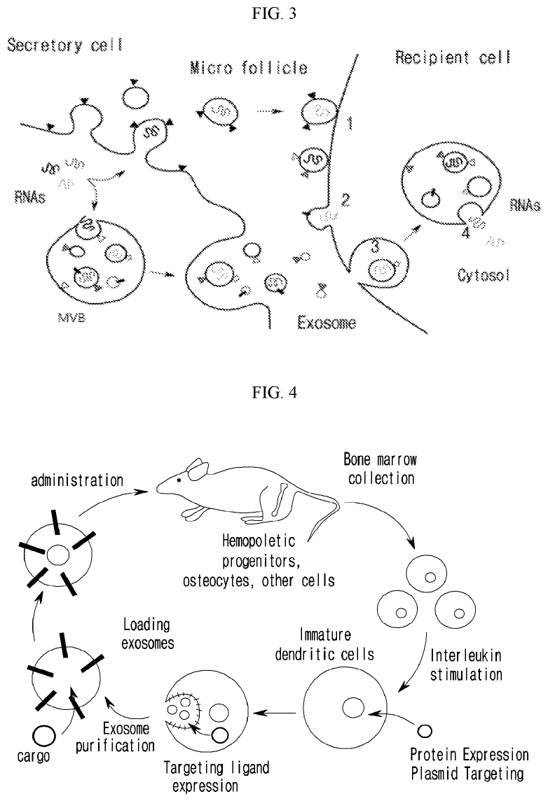

FIG. 13 illustrates the expression of the Luciferase-mCherry fusion protein measured by fluorescence imaging (a) and the luciferase activity (b) and the number of molecules in the production cells (c):
Control: HEK293T cells treated with nothing;
OVER: HEK293T cells introduced with Luciferase-mCherry-CRY2 alone;
XP: HEK293T cells introduced with XPACK-Luciferase-mCherry by using XPACK (Systems Biosciences), the commercial vector designed for exosome loading technique;
EXPLOR: HEK293T cells introduced with Luciferase-mCherry-CRY2 and CIBN-EGFP-CD9 according to the present invention.

FIG. 14 illustrates the luciferase activity (a) and the number of molecules (b) in the produced exosome:
NEG: exosome produced in the HEK293T cells treated with nothing;
OVER: exosome produced in the HEK293T cells introduced with Luciferase-mCherry-CRY2;
XP: exosome produced in the HEK293T cells introduced with XPACK-Luciferase-mCherry by using XPACK (Systems Biosciences), the commercial vector designed for exosome loading technique;
EXPLOR: exosome produced in the HEK293T cells introduced with Luciferase-mCherry-CRY2 and CIBN-EGFP-CD9 according to the present invention;
ON: exosome produced by culturing under the irradiation of 200 μW blue light for 72 hours,
OFF: exosome produced by culturing under the light-free condition for 72 hours.

FIG. 15 illustrates the loading efficiency of a cargo protein in the exosome produced above.

FIG. 16 illustrates the transfer efficiency of a cargo protein into the target cells (HeLa) using exosome:
Control: exosome produced in the HEK293T cells treated with nothing;
OVER: exosome produced in the HEK293T cells introduced with Luciferase-mCherry-CRY2;
XP: exosome produced in the HEK293T cells introduced with XPACK-Luciferase-mCherry by using XPACK (Systems Biosciences), the commercial vector designed for exosome loading technique;
EXPLOR: exosome produced in the HEK293T cells introduced with Luciferase-mCherry-CRY2 and CIBN-EGFP-CD9 according to the present invention;
ON: exosome produced by culturing under the irradiation of 200 μW blue light for 72 hours,
OFF: exosome produced by culturing under the light-free condition for 72 hours.

FIG. 17 illustrates the location of the expression of Luciferase-mCherry-CRY2 and CIBN-EGFP-CD9 in HEK293T cells, indicating they share the same position for the expression.

FIG. 18 illustrates the location of the expression of Cre-mCherry-CRY2 and CIBN-EGFP-CD9 in HEK293T cells, indicating they share the same position for the expression.

FIG. 19A illustrates that treatment with Cre: EXPLOR induced the expression of ZsGreen in HT1080 cells transiently transfected with pCAG-loxP-STOP-loxP-ZsGreen (Scale bars, 40 μm):
Negative: EXPLOR: no cre-loaded exosome as negative control;
Cre: EXPLOR: Cre-loaded exosome; and
pcMV-Cre: pCMV-Cre vector transfection as positive control.

FIG. 19B illustrates that treatment with Cre: EXPLOR induced the expression of ZsGreen in HeLa cells transiently transfected with pCAG-loxP-STOP-loxP-ZsGreen (Scale bars, m):
Negative: EXPLOR: no Cre-loaded exosome as negative control;
Cre: EXPLOR: Cre-loaded exosome; and
pcMV-Cre: pCMV-Cre vector transfection as positive control.

FIG. 20 illustrates that treatment with Cre: EXPLOR induced the expression of ZsGreen in primary rat embryonic neuron transiently transfected with pCAG-loxP-STOP-loxP-ZsGreen (Scale bars, 100 μm):
Control: EXPLOR: no Cre-loaded exosome as negative control; and
Cre: EXPLOR::Cre-loaded exosome.

FIG. 21 illustrates that treatment with Cre:EXPLOR induced the expression of ZsGreen in transgenic mice having pCAG-loxP-STOP-loxP-eNpHR3.0-EYFP gene (Scale bars, 500 μm):
Control: EXPLOR: no Cre-loaded exosome as negative control;
Cre: EXPLOR: Cre-loaded exosome;
Hip: hippocampus; and
Th: thalamus.

FIG. 22 illustrates the results of immunohistochemistry for NEuN/GFAP in transgenic mice having pCAG-loxP-STOP-loxP-eNpHR3.0-EYFP gene
Pink: neuronal-specific nuclear protein; NEuN, positive neurons; and
Red: glial fibrillary acidic protein; GFAP, positive astrocyte cells.
Objective lens, 40×. Scale bar, 20 m.

FIG. 23 illustrates the location of the expression of Cas9-mCherry-CRY2 and CIBN-EGFP-CD9 in HEK293T cells, indicating they share the same position for the expression.

FIG. 24 illustrates the generation of DNA constructs used for the production of Cas9-loaded exosome.

FIG. 25 illustrates the results of measuring the content of a cargo protein (CRISPR-Cas9 protein) captured in exosome.

FIG. 26 illustrates the location of the expression of GBA-mCherry-CRY2 and CIBN-EGFP-CD9 in HEK293T cells, indicating they share the same position for the expression.

FIG. 27 illustrates the expression of endogenous GBA and GBA-mcherry-CRY2 fusion protein in HEK293T cell transiently transfected with GBA-mCh-CRY2 and CIBN-EGFP-CD9, rat primary astrocyte, human primary astrocyte and Gaucher fibroblast.

FIG. 28 illustrates the results of measuring the content of a cargo protein (GBA protein) captured in exosome.

FIG. 29 illustrates the results of measuring the enzymatic activity of 3-glucocerebrosidase, a cargo protein (GBA protein) captured in exosome.
Exo-Naive: HEK293T-derived exosome
Exo-GBA: exosome including β-glucocerebrosidase

FIG. 30 illustrates the results of treatment of GBA-exosomes to Gaucher disease patient-derived fibroblasts, indicating treatment with GBA-exosomes significantly induced the enzymatic activity in β-glucocerebrosidase-deficient cells.

FIG. 31 illustrates the generation of DNA constructs used for the production of PTEN-loaded exosome and cells stably expressing PTEN-loaded exosome.

FIG. 32 illustrates the location of the expression of luciferase-mCherry-CRY2 and CIBN-EGFP-CD9 in HEK293T cells, indicating they share the same position for the expression.

FIG. 33 illustrates the results of measurement of quantitative luciferase activity based on the number of luciferase molecules.

FIG. 34 illustrates the location of the expression of PrxI/II-mCherry-CRY2 and CIBN-EGFP-CD9 in HEK293T cells, indicating they share the same position for the expression.
Prx I: peroxiredoxin I
Prx II: peroxiredoxin II

FIG. 35 illustrates the protective effect of PrxI/II-loaded exosomes in H₂O₂-induced oxidative stress and cytotoxicity.
None: H₂O₂-treated group;
Cre: EXPLOR: Cre-loaded exosome
Prx I: EXPLOR: PrxI-loaded exosomes; and
Prx II: EXPLOR: PrxII-loaded exosomes.

FIG. 36 illustrates the location of the expression of MyoD-mCherry-CRY2 and CIBN-EGFP-CD9 in HEK293T cells, indicating they share the same position for the expression.

FIG. 37 illustrates the results of treatment of MyoD-loaded exosomes to adipose-derived stem cells and, indicating treatment with MyoD-exosomes (clone #A6) induced the proliferation of cells after 6 days.

FIG. 38 illustrates the generation of cells stably expressing p53-loaded exosome.

FIG. 39 illustrates the results of measuring the content of a cargo protein (p53 protein) captured in exosome.
Stable cell: cells stably expressing p53-loaded exosome.
mCherry: mCherry-loaded exosome
p53: p53-loaded exosome

FIG. 40 illustrates the results of measurement of transcriptional activity of p53 using luciferase reporter gene, indicating treatment with p53-loaded exosomes induced transcriptional activity of p53 in doxorubicin-treated HeLa cells.

FIG. 41 illustrates the generation of DNA constructs used for the production of HMGB1-loaded exosome and cells stably expressing HMGB1-loaded exosome.

FIG. 42 illustrates the location of the expression of srIκB-mCherry-CRY2 and CIBN-EGFP-CD9 in HEK293T cells, indicating they share the same position for the expression.

FIG. 43 illustrates that treatment with srIkB-mCherry:EXPLORs significantly reduced tumor necrosis factor-α-induced translocation and DNA binding of the p65 subunit of NF-κB in HeLa cells.

FIG. 44 illustrates the analysis of disease progression after administration of srIkB-loaded exosomes to rheumatoid arthritis animal model.

FIG. 45 illustrates the survival curve of groups treated with srIkB-loaded exosomes in LPS-induced sepsis model.
No exosome: only LPS treated group
Naive exosome: group treated with HEK293T-derived exosome
srIkB exosome: group treated with srIkB-loaded exosomes

FIG. 46 illustrates the location of the expression of pYSTAT3 intrabody-mCherry-CRY2 and CIBN-EGFP-CD9 in HEK293T cells, indicating they share the same position for the expression.

FIG. 47 illustrates the intracellular delivery of pYSTAT3 intrabody to target cells using pYSTAT3 intrabody-loaded exosomes.

FIG. 48 illustrates location of the expression of Bax-mCherry-CRY2 and CIBN-EGFP-CD9 in HEK293T cells, indicating they share the same position for the expression.

FIG. 49 illustrates that treatment with Bax-loaded exosome induced a rapid release of cytochrome c from the mitochondria in HeLa cells FIG. 50 illustrates location of the expression of AIMP-mCherry-CRY2 and CIBN-EGFP-CD9 in HEK293T cells, indicating they share the same position for the expression.

FIG. 51 illustrates the results of measuring the content of a cargo protein (AIMP protein) captured in exosome.

FIG. 52 illustrates location of the expression of mCherry-CRY2 and CIBN-EGFP-CD9 in HEK293T cells, indicating they share the same position for the expression.

FIG. 53 illustrates DNA deletion by Cre recombinase.

FIG. 54 illustrates difference between Cas9 and Cpf1 protein.

### DETAILED DESCRIPTION OF THE INVENTION

The present invention provides compositions containing exosome loaded with a cargo protein.

In another embodiment, the present invention provides a method for preparing the exosome loaded with a cargo protein using a photo-specific binding protein.

In a further embodiment, the present invention provides a method of delivering the cargo protein to cytosol using the exosome.

In another embodiment, the present invention provides a method for the mass-production of exosome containing a fusion protein composed of an exosome specific marker and a cargo protein.

The present invention provides a method for the mass-production of exosome containing a cargo protein separated from the membrane of exosome by using a photo-specific binding protein pair.

The present invention also provides a vector for preparing exosome which is usable for the preparation of the exosome.

The present invention further provides a method to introduce a cargo protein in cytosol by using the exosome above.

In one embodiment, the present invention provides pharmaceutical compostions containing exosomes loaded with a cargo proteins and a method for preparing the same.

In a preferred embodiment, the cargo protein is super-repressor-IκB protein inhibiting NF-κB, Bax (Bcl-2-associated X protein), Peroxiredoxin I, Peroxiredoxin II, Cre recombinase, Cas9 (CRISPR associated protein 9), Cpf1 (CRISPR from Prevotella and Francisella 1) or GBA (β-glucocerebrosidase).

The present invention provides exosome comprising a cargo protein which can be used for the treatment of various diseases in vivo by delivering the cargo protein. For example, exosome can be prepared to include a protein or siRNA having an anticancer activity and then treated to cancer cells for cancer treatment (FIG. 4).

For the exosomes containing a cargo protein used for the treatment of disease, the exosomes needs to be prepared efficiently to have proper load of the cargo protein. Korean Patent Publication No. 2004-0015508 describes a method for preparing exosome comprising a specific antigen. Precisely, it describes a method of discharging a cargo protein by using exosome, wherein a gene encoding a specific antigen is inserted in a host cell line and a protein of the introduced gene is stably expressed in the cell line which is discharged extracellularly through exosome, and a method using the exosome as a vaccine.

However exosome is formed naturally within the cells. So, even though a gene encoding a cargo protein is inserted in the cell producing exosome endogenously, it is very difficult to prepare exosome comprising the expressed protein in it thereby.

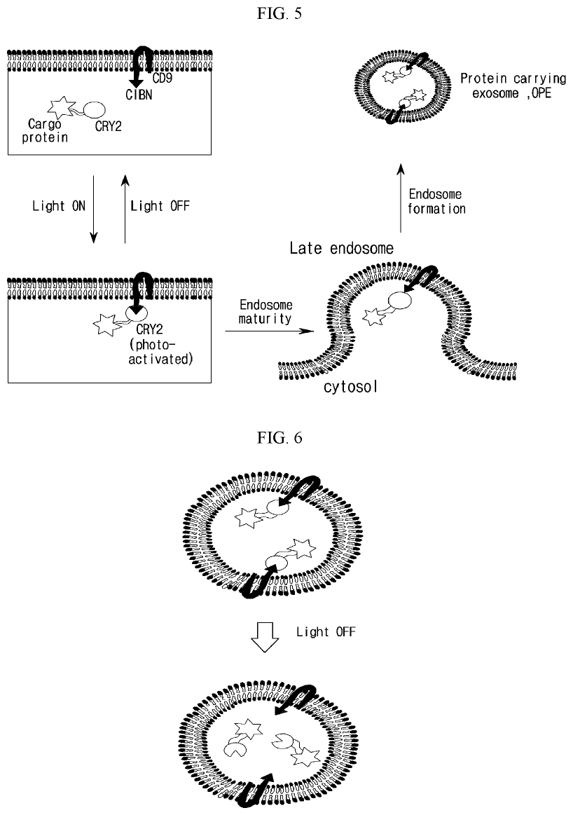

The present invention provides methods for preparing exosome comprising a cargo protein more efficiently. As a result, the inventors succeeded in preparing exosome comprising a cargo protein efficiently by expressing a fusion protein composed of an exosome specific marker and a cargo protein massively in the cell producing exosome endogenously at a high concentration (FIG. 5).

The cargo protein is attached on the membrane of exosome, according to the method above. So, the fusion protein composed of a pair of an exosome specific marker and a cargo protein is expressed in the cell producing exosome at a high concentration, followed by irradiation to induce the linkage of the fusion protein. Then, the fusion protein is introduced inside the exosome by the action of the exosome specific marker. When the irradiation is terminated after the introduction, the fusion protein is separated into a cargo protein and a photo-specific binding protein inside the exosome. As a result, the exosome containing a free cargo protein separated from the fusion protein can be prepared efficiently (FIG. 6).

The cargo proteins loaded in the exosome in the present invention includes, but not limited to, natural or non-natural proteins, truncated form or mutated form. Examples of the cargo proteins are listed, but not limited to, in the following table.

TABLE 1

| Classification | Sub-Class | Example |
|---|---|---|
| Enzymes | Proteases | MMPs and TIMP (tissue |
| | (extracellular & | inhibitor metalloproteases) |
| | intracellular) and | Caspases and their inhibitors |
| | their inhibitors | Cathepsins and their inhibitors |
| | Nucleases | Cre recombinase |
| | | CRISPR/cas9 |
| | | Caspase-activated DNase |
| | hydrolytic enzymes | Lysosomal enzymes including |
| | | Beta-glucocerebrosidase |
| | Kinases and | Mitogen activated kinases: |
| | phosphatase | p38 MAP kinase |
| | | Inhibitor kappa B kinase (IKK) |
| | | PTEN phosphatase |
| | | Janus kinase |
| | others | Ubiquitin ligase |
| | | luciferase |
| | | peroxiredoxins |
| Transcription | Transcription | NF-kB/super repressor IkB |
| factors | factors and | MyoD |
| | their inhibitors | Tbx18 (T-box transcription |
| | | factor 18) |
| | | p53 |
| | | HMGB1 (High mobility |
| | | group box 1 protein) |
| Antibodies | Antibodies and | pYSTAT3 intrabody |
| | associated peptides | |
| others | unclassified | Pro-apoptotic proteins: Bax |
| | | Anti-apoptotic proteins: BcL-xL |
| | | Multifunctional signal |
| | | molecules: AIMP |
| | | (Aminoacyl-tRNA synthetase- |
| | | interacting multifunctional |
| | | proteins) |
| | | Fluorescent proteins |
| | | (mCherry, GFP) |
| | | Nucleic acid-binding |
| | | proteins (ex. RNPs) |

<Enzymes>

Enzymes are biological catalytic molecules accelerating chemical reactions in living organisms. Enzymes bind to their substrates and facilitate the reaction rate by lowering its activation energy. Enzymes can be classified as follows; proteases, nucleases, hydrolytic enzymes, kinases, phosphatase and other types of enzymes.

The target proteins loaded in the exosomes in the present invention include enzymes and their regulators. Examples of the target proteins are listed, but not limited to, in the following description.

—Proteases and their Inhibitors

MMPs and TIMP

Matrix metalloproteinases (MMPs), also known as matrixins, are calcium-dependent zinc-containing endopeptidases. MMPs are capable of degrading all kinds of extracellular matrix proteins and known to be involved in the cleavage of cell surface receptors, the release of apoptotic ligands (such as the FAS ligand), and chemokine/cytokine inactivation. MMPs are also thought to play a major role in cell behaviors such as cell proliferation, migration (adhesion/dispersion), differentiation, angiogenesis, apoptosis, and host defense.

The matrix metalloproteinases are inhibited by specific endogenous tissue inhibitors of metalloproteinases (TIMPs), which comprise a family of four protease inhibitors: TIMP1, TIMP2, TIMP3 and TIMP4.

The balance of MMPs and TIMPs plays an important role in tissue remodeling associated with various physiological or pathological processes such as morphogenesis, angiogenesis, tissue repair, cirrhosis, arthritis, and metastasis. MMP-2 and MMP-9 are thought to be important in metastasis. MMP-1 is thought to be important in rheumatoid arthritis and osteoarthritis. Dysregulation of the balance between MMPs and TIMPs is also a characteristic of acute and chronic cardiovascular diseases.

The exosomes comprising MMPs and TIMPs are prepared by expressing a fusion protein composed of an exosome specific marker and a target protein massively in the cell producing the exosomes at a high concentration. The exosomes loaded with MMPs or TIMPs can be used to treat MMP-associated diseases including rheumatoid arthritis.

Caspases and their Inhibitors

Caspases (cysteine-aspartic proteases, cysteine aspartases or cysteine-dependent aspartate-directed proteases) are a family of protease enzymes playing essential roles in programmed cell death including apoptosis, pyroptosis and necroptosis. These forms of cell death are important for protecting an organism from stress signals and pathogenic attack. Caspases also have a role in inflammation, whereby it directly processes pro-inflammatory cytokines such as pro-IL1β. These are signaling molecules that allow recruitment of immune cells to an infected cell or tissue. There are other identified roles of caspases such as cell proliferation, tumor suppression, cell differentiation, neural development and axon guidance and ageing.

Caspase deficiency has been identified as a cause of tumor development. Tumor growth can occur by a combination of factors, including a mutation in a cell cycle gene which removes the restraints on cell growth, combined with mutations in apoptotic proteins such as Caspases that would respond by inducing cell death in abnormally growing cells.

Conversely, over-activation of some caspases such as caspase-3 can lead to excessive programmed cell death. This is seen in several neurodegenerative diseases where neural cells are lost, such as Alzheimer's disease. Caspases involved with processing inflammatory signals are also implicated in disease. Insufficient activation of these caspases can increase an organism's susceptibility to infection, as an appropriate immune response may not be activated. The integral role caspases play in cell death and disease has led to research on using caspases as a drug target. For example, inflammatory caspase-1 has been implicated in causing autoimmune diseases.

The exosomes comprising caspases and their inhibitors are prepared by expressing a fusion protein composed of an exosome specific marker and a target protein massively in the cell producing the exosomes at a high concentration. The exosomes loaded with caspases or their inhibitors can be used to treat caspase-associated diseases including neurodegenerative diseases or autoimmune diseases.

Cathepsins and their Inhibitors

Cathepsins are proteases found in all animals as well as other organisms. There are approximately a dozen members of this family, which are distinguished by their structure, catalytic mechanism, and which proteins they cleave. Most of the members become activated at the low pH found in lysosomes. Thus, the activity of this family lies almost entirely within those organelles.

Cathepsins have been implicated in cancer, stroke, Alzheimer's disease, arthritis, Ebola, COPD, chronic periodontitis, pancreatitis and several ocular disorders including keratoconus. Especially for cancer, cathepsin D is a mitogen and it attenuates the anti-tumor immune response of decaying chemokines to inhibit the function of dendritic cells. Cathepsin B and L are involved in matrix degradation and cell invasion.

The exosomes comprising cathepsins and their inhibitors are prepared by expressing a fusion protein composed of an exosome specific marker and a target protein massively in the cell producing the exosomes at a high concentration. The exosomes loaded with cathepsins or their inhibitors can be used to treat varying cathepsin-associated diseases including cancer and Alzheimer's disease.

Nucleases

Cre Recombinase

Cre recombinase is the protein isolated from P1 bacteriophage, and induces recombination by detecting two different loxP region. The loxP is DNA fragment with 34 bp and is composed of two 13 bp palindromic sequence on both extremes and 8 bp asymmetrical core spacer on middle. The Cre recombinase binds to palindromic sequence, change the spacer region of DNA after cutting, and then recombine DNA (FIG. 53). Excision or inversion of DNA sequence between two different lowP regions based on the directionality of spacer. The excision or inversion is occurred if direction of lowP region is same or reverse, respectively.

One of the representative examples of Cre recombinase utilization is the conditional knockout mouse which can inhibit mutated period and expressed tissues of specific gene. This technology is that eliminating specific target gene in some isolated cells by producing loxP inserted mouse between front and end of specific target gene, mating with Cre-expressing transgenic mouse, or directly treating Cre recombinase to specific cell. The conditional knockout mouse is efficient to confirm the function of specific gene by expressing such gene, which is lethal in early phase of embryo development, in late phase of embryo development or adult.

The present invention provides exosomes loaded with Cre recombinase protein and confirmed that Cre recombinase protein was delivered to the cytosol of the target cells. The results indicate that the exosome of the present invention loaded with Cre recombinase protein can be used for conditional gene manipulation.

CRISPR/Cas9

CRISPR-Cas9 is an RNA-based artificial restriction enzyme that makes DNA correction be possible by restricting specific region of genes. Recently, it is remarkably spotlighted as the key element of genetic engineering.

CRISPR, which is kind of palindromic sequence, is the abbreviated form of Clustered regularly-interspaced short palindromic repeats and first observed acquired immunity system of bacterium. Firstly, Cas9 protein recognizes and restricts invaded virus. Then the restricted virus sequence is inserted into CRISPR sequence, and combined virus and CRISPR sequence is transcribed as RNA. This RNA is used in formation of Cas9 complex. After this process, transcribed ‘CRISPR+virus sequence’ is combined with Cas9 and eliminates same invaded virus faster than Cas9 alone. This mechanism can be applied in genetic engineering by combining target sequence with Cas9 complex to restrict target sequence.

Cpf1 is protein with similar function with Cas9 protein from aforementioned engineered endonuclease CRISPR-Cas9 system. As shown in FIG. 54, Cpf1 recognizes protospace adjacent motif (PAM) sequence unlike Cas9. It can be used on the region unrecognized by Cas9, and especially it is more practical because short crispr RNA (crRNA) alone can be worked. In case of Cas9, tracrRNA is additionally needed.

The present invention provides exosomes loaded with Cas9 or Cpf1 protein and confirmed that Cas9 or Cpf1 protein was delivered to the cytosol of the target cells. The results indicate that the exosome of the present invention loaded with Cas9 or Cpf1 protein can be used for removing, adding or altering sections of the DNA sequence.

Caspase-Activated DNase

Caspase-Activated DNase (CAD) or DNA fragmentation factor subunit beta (DFFB) is a protein that is encoded by the DFFB gene in humans. It breaks up the DNA during apoptosis and promotes cell differentiation. It is usually an inactive monomer inhibited by ICAD. This is cleaved before dimerization.

Apoptosis is a cell death process that removes toxic and/or useless cells during mammalian development. The apoptotic process is accompanied by shrinkage and fragmentation of the cells and nuclei and degradation of the chromosomal DNA into nucleosomal units. DNA fragmentation factor (DFF) is a heterodimeric protein of 40-kD (DFFB) and 45-kD (DFFA) subunits. DFFA is the substrate for caspase-3 and triggers DNA fragmentation during apoptosis. DFF becomes activated when DFFA is cleaved by caspase-3. The cleaved fragments of DFFA dissociate from DFFB, the active component of DFF. DFFB has been found to trigger both DNA fragmentation and chromatin condensation during apoptosis.

The exosomes comprising Caspase-activated DNase are prepared by expressing a fusion protein composed of an exosome specific marker and a target protein massively in the cell producing the exosomes at a high concentration. The exosomes loaded with Caspase-activated DNase can be used to regulate apoptosis in diverse systems.

Hydrolytic Enzymes

Lysosomal Enzyme Including Beta-Glucocerebrosidase

Lysosomal storage disorder is the disease because of storage of materials degraded by lysosome according to the innate deficiency of lysosome. One of the common lysosomal storage disorder is Gaucher disease which is induced by genetic deficiency of β-glucocerebrosidase (GBA), lysosomal enzyme.

Lack of GBA induces malfunction on liver, spleen, and bone marrow, and so on by storing glucocerebrosidase/glucosylsphingosine on lysosome of macrophage. Also it induces hematologic abnormality such as anemia, thrombocytopenia, and leukopenia, gepatolientalny, osteoclasia, and central nerve injury, etc.

Present treatment of Gaucher disease is the enzyme replacement therapy injecting GBA analogue, cerezyme, by intravenous injection. However, these kinds of protein drugs have various disadvantage such as short half-life in blood, low efficiency because of antibody production, difficulty of delivery to lysosome, and the impossibility on applying neurogenic Gaucher disease, etc.

The present invention provides exosomes loaded with GBA (β-glucocerebrosidase) protein and confirmed that GBA (β-glucocerebrosidase) protein was delivered to the cytosol of the target cells. The results indicate that the exosome of the present invention loaded with GBA (β-glucocerebrosidase) protein can be used for treatment of Gaucher disease.

Kinases and Phosphatase

Mitogen Activated Kinases: p38 MAP Kinase

P38 mitogen-activated protein kinases are a class of mitogen-activated protein kinases (MAPKs) that are responsive to stress stimuli, such as cytokines, ultraviolet irradiation, heat shock, and osmotic shock. P38 MAP kinase are involved in cell differentiation, apoptosis and autophagy.

P38 MAP Kinase (MAPK) participates in a signaling cascade controlling cellular responses to cytokines and stress. P38 inhibitors are being sought for possible therapeutic effect on autoimmune diseases and inflammatory processes.

The exosomes comprising p38 MAPK and its inhibitor are prepared by expressing a fusion protein composed of an exosome specific marker and a target protein massively in the cell producing the exosomes at a high concentration. The exosomes loaded with p38 MAPK or its inhibitors can be used to treat p38 MAPK-associated diseases including autoimmune diseases.

Inhibitor Kappa B Kinase (IKK)

The IκB kinase (IKK) is an enzyme complex that is involved in propagating the cellular response to inflammation. The IκB kinase enzyme complex is part of the upstream NF-κB signal transduction cascade. The IκBα (inhibitor of kappa B) protein inactivates the NF-κB transcription factor by masking the nuclear localization signals (NLS) of NF-κB proteins and keeping them sequestered in an inactive state in the cytoplasm. IKK phosphorylates the inhibitory IκBα protein. This phosphorylation results in the dissociation of IκBα from NF-κB. NF-κB, which is now free, migrates into the nucleus and activates the expression of at least 150 genes; some of which are anti-apoptotic.

IκB kinase activity is essential for activation of members of the nuclear factor-kB (NF-κB) family of transcription factors, which play a fundamental role in lymphocyte immune-regulation. Activation of the canonical NF-κB pathway begins in response to stimulation by various pro-inflammatory stimuli, including lipopolysaccharide (LPS) expressed on the surface of pathogens, or the release of pro-inflammatory cytokines such as tumor necrosis factor (TNF) or interleukin-1 (IL-1). Following immune cell stimulation, a signal transduction cascade leads to the activation of the IKK complex, an event characterized by the binding of NEMO to the homologous kinase subunits IKK-α and IKK-β.

Though functionally adaptive in response to inflammatory stimuli, deregulation of NF-κB signaling has been exploited in various disease states. Increased NF-κB activity as a result of constitutive IKK-mediated phosphorylation of IκBα has been observed in the development of atherosclerosis, asthma, rheumatoid arthritis, inflammatory bowel diseases, and multiple sclerosis. Specifically, constitutive NF-κB activity promotes continuous inflammatory signaling at the molecular level that translates to chronic inflammation phenotypically. Furthermore, the ability of NF-κB to simultaneously suppress apoptosis and promote continuous lymphocyte growth and proliferation explains its intimate connection with many types of cancer.

The exosomes comprising IKK are prepared by expressing a fusion protein composed of an exosome specific marker and a target protein massively in the cell producing the exosomes at a high concentration. The exosomes loaded with IKK can be used to treat NF-κB-associated diseases including cancers.

PTEN Phosphatase

Phosphatase and tensin homolog (PTEN) is identified as a tumor suppressor protein. Mutations of this gene are a step in the development of many cancers. The protein contains a tensin-like domain as well as a catalytic domain similar to that of the dual specificity protein tyrosine phosphatases. Unlike most of the protein tyrosine phosphatases, this protein preferentially dephosphorylates phosphoinositide substrates. It negatively regulates intracellular levels of phosphatidylinositol-3, 4, 5-trisphosphate in cells and functions as a tumor suppressor by negatively regulating Akt/PKB signaling pathway.

PTEN loss or mutation is closely related with cancer, non-cancerous neoplasia and autism. Especially during tumor development, mutations and deletions of PTEN occur that inactivate its enzymatic activity leading to increased cell proliferation and reduced cell death. Frequent genetic inactivation of PTEN occurs in glioblastoma, endometrial cancer, and prostate cancer; and reduced expression is found in many other tumor types such as lung and breast cancer. Furthermore, PTEN mutation also causes a variety of inherited predispositions to cancer.

Mutations in the PTEN gene cause several other disorders that, like Cowden syndrome, are characterized by the development of non-cancerous tumors called hamartomas. These disorders include Bannayan-Riley-Ruvalcaba syndrome and Proteus-like syndrome. Together, the disorders caused by PTEN mutations are called PTEN hamartoma tumor syndromes, or PHTS. Mutations responsible for these syndromes cause the resulting protein to be non-functional or absent. The defective protein allows the cell to divide in an uncontrolled way and prevents damaged cells from dying, which can lead to the growth of tumors.

The exosomes comprising PTEN are prepared by expressing a fusion protein composed of an exosome specific marker and a target protein massively in the cell producing the exosomes at a high concentration. The exosomes loaded with PTEN can be used to treat varying types of cancers.

Janus Kinase

Janus kinase (JAK) is a family of intracellular, nonreceptor tyrosine kinases that transduce cytokine-mediated signals via the JAK-STAT pathway. Since members of the type I and type II cytokine receptor families possess no catalytic kinase activity, they rely on the JAK family of tyrosine kinases to phosphorylate and activate downstream proteins involved in their signal transduction pathways. After the receptor associates with its respective cytokine/ligand, it goes through a conformational change, bringing the two JAKs close enough to phosphorylate each other. The JAK auto-phosphorylation induces a conformational change within itself, enabling it to transduce the intracellular signal by further phosphorylating and activating transcription factors called STATs (Signal Transducer and Activator of Transcription). The activated STATs dissociate from the receptor and form dimers before translocating to the cell nucleus, where they regulate transcription of selected genes.

Some examples of the molecules that use the JAK/STAT signaling pathway are colony-stimulating factor, prolactin, growth hormone, and many cytokines. JAK inhibitors are under development for the treatment of psoriasis, rheumatoid arthritis, polycythemia vera, alopecia, essential thrombocythemia, ulcerative colitis, myeloid metaplasia with myelofibrosis and vitiligo.

The exosomes comprising JAK and its inhibitors are prepared by expressing a fusion protein composed of an exosome specific marker and a target protein massively in the cell producing the exosomes at a high concentration. The exosomes loaded with JAK or its inhibitors can be used to treat JAK-associated diseases including cancers.

Others

Ubiquitin Ligase

A ubiquitin ligase (also called an E3 ubiquitin ligase) is a protein that recruits an E2 ubiquitin-conjugating enzyme that has been loaded with ubiquitin, recognizes a protein substrate, and assists or directly catalyzes the transfer of ubiquitin from the E2 to the protein substrate. The ubiquitin is attached to a lysine on the target protein by an isopeptide bond. E3 ligases interact with both the target protein and the E2 enzyme, and so impart substrate specificity to the E2.

Ubiquitination by E3 ligases regulates diverse areas such as cell trafficking, DNA repair, and signaling and is of profound importance in cell biology. E3 ligases are also key players in cell cycle control, mediating the degradation of cyclins, as well as cyclin dependent kinase inhibitor proteins.

E3 ubiquitin ligases regulate homeostasis, cell cycle, and DNA repair pathways, and as a result, a number of these proteins are involved in a variety of cancers, including famously MDM2, BRCA1, and Von Hippel-Lindau tumor suppressor. For example, a mutation of MDM2 has been found in stomach cancer, renal cell carcinoma, and liver cancer (amongst others) to deregulate MDM2 concentrations by increasing its promoter's affinity for the Sp1 transcription factor, causing increased transcription of MDM2 mRNA.

The exosomes comprising Ubiquitin ligase are prepared by expressing a fusion protein composed of an exosome specific marker and a target protein massively in the cell producing the exosomes at a high concentration. The exosomes loaded with Ubiquitin ligase can be used to treat ubiquitination-associated diseases including cancers.

Luciferase

Luciferase is a generic term for the class of oxidative enzymes that produce bioluminescence, and is usually distinguished from a photoprotein. Luciferases are widely used in biotechnology, for microscopy and as reporter genes, for many of the same applications as fluorescent proteins. However, unlike fluorescent proteins, luciferases do not require an external light source, but do require addition of luciferin, the consumable substrate.

All luciferases are classified as oxidoreductases (EC 1.13.12.—), meaning they act on single donors with incorporation of molecular oxygen. Because luciferases are from many diverse protein families that are unrelated, there is no unifying mechanism, as any mechanism depends on the luciferase and luciferin combination. However, all characterized luciferase-luciferin reactions to date have been shown to require molecular oxygen at some stage.

In biological research, luciferase is commonly used as a reporter to assess the transcriptional activity in cells that are transfected with a genetic construct containing the luciferase gene under the control of a promoter of interest. Additionally, pro-luminescent molecules that are converted to luciferin upon activity of a particular enzyme can be used to detect enzyme activity in coupled or two-step luciferase assays. Such substrates have been used to detect caspase activity and cytochrome P450 activity, among others. Luciferase can also be used to detect the level of cellular ATP in cell viability assays or for kinase activity assays. Luciferase can act as an ATP sensor protein through biotinylation. Biotinylation will immobilize luciferase on the cell-surface by binding to a streptavidin-biotin complex. This allows luciferase to detect the efflux of ATP from the cell and will effectively display the real-time release of ATP through bioluminescence. Luciferase can additionally be made more sensitive for ATP detection by increasing the luminescence intensity by changing certain amino acid residues in the sequence of the protein.

Whole animal imaging (referred to as in vivo or, occasionally, ex vivo imaging) can be performed using luciferase-expressing cell line injection. Different types of cells (e.g. bone marrow stem cells, T-cells) can be engineered to express a luciferase allowing their non-invasive visualization inside a live animal using a sensitive charge-couple device camera (CCD camera). This technique has been used to follow tumorigenesis and response of tumors to treatment in animal models.

The present invention prepared exosomes loaded with luciferase protein and confirmed that luciferase protein was delivered to the cytosol of the target cells. The results indicate that the exosome of the present invention loaded with luciferase protein can be used for cell viability assay, kinase activity assay and whole animal imaging.

Peroxiredoxins

Peroxiredoxin (Prx) is representative antioxidant enzyme in cytoplasm and obtain 0.1 0.8% of water-soluble protein in mammalian cells. Prx has the role to reduce hydroperoxide to H₂O and ROH— by receiving 2e− in cells. Prx also involves in cell proliferation, differentiation, death, and cell signal transduction by participating the formation and elimination of H₂O₂ (nmol concentration). Prx is classified more specifically into 1-Cys Prx, or 2-Cys Prx based on the number of cysteine amino acid. Furthermore, 2-Cys prx is subdivided into ‘typical’ or ‘atypical’ based on structural, and mechanistic difference. All three Prx have difference in oxidation-reduction from second process of formation of Cys-SOH. Prx I-Prx IV are typical 2-Cys Prx, and Prx V is atypical 2-Cys Prc, and Prx VI is 1-Cys Prx. Some cases of 2-Cys Prx form oligomer.

Prx I, and II involve in activation of receptor-signaling pathway by regulating the concentration of H₂O₂ in cell generated by growth factor and TNF-α. Specifically, Prx II has the role in protecting cells from stimulus of cell-death inducing factor such as serum starvation, ceramide, and etoposide.

In normal cells, Prx I have the role to maintain activity of PTEN phosphatase by inhibiting its oxidation. However, in case of increased oxidative stress, the activity of PTEN is inhibited by H₂O₂ through separation of Prx from PTEN by irreversible oxidation. Consequently, it induces tumor through continuous activation of cell proliferating signal such as Akt.

It has a significant relation with disease that quantitative change of Prx in cell. During the process of cancer development, arteriosclerosis, respiratory inflammation, osteoporosis, obesity, and degenerative dementia, quantitative change of reactive oxygen species has a close connection.

The present invention provides exosomes loaded with Peroxiredoxin I or Peroxiredoxin II protein and confirmed that Peroxiredoxin I or Peroxiredoxin II protein was delivered to the cytosol of the target cells. The results indicates that the exosome of the present invention loaded with Peroxiredoxin I or Peroxiredoxin II protein can be used for treatment of reactive oxygen-related diseases.

<Transcription Factors>

Transcription factors are proteins regulating mRNA transcription from DNA in eukaryotes. Transcription factors are associated with the basal transcription regulation, organism development, response to intercellular signals or environment, cell cycle control and pathogenesis.

The target proteins loaded in the exosomes in the present invention include transcription factors and their regulators (enhancers or inhibitors). Examples of the target proteins are listed, but not limited to, in the following description.

Transcription Factors and their Regulators

NF-kB Regulator, Super-Repressor IkB

NF-κB is the major transcription factor inducing the inflammatory response, and regulates the expression of inflammatory-related genes in various types of cells especially immune cells. Therefore, it can be effective therapeutic strategy for incurable chronic inflammatory disease such as rheumatoid arthritis, sepsis, and psoriasis that selectively inhibits the overactive NF-κB signaling pathway in immune cells. In addition, activation of NF-κB has the role that inhibits apoptosis by increasing the expression of anti-apoptotic factors. From this role, continuous activation of NF-κB signaling pathway in cancer is the cause for anticancer drug resistance and then decreases the therapeutic effects of anticancer drugs.

Most NF-κB is on inactive phase by binding with IκB, which is the inhibitory protein of NF-κB, in normal cells. IκB Kinase (IKK) complex activated by various stimuli such as TNF-α and LPS phosphorylates IκB. The phosphorylated IκB is then ubiquitinated and finally degraded by proteasome. Through degradation of IκB, NF-κB (p50/p65) bound on IκB passes through nuclear membrane. After passing, it activates mRNA transcription by binding on the promotor region of target genes in nucleus. This is the important element of immune response that induces transcription of cytokine and inflammatory mediator such as iNOS, COX-2, NO, PGE2, TNF-α, and IL-1 (Lappas et al., Biol. Reprod. 67:668673, 2002).

Super-repressor IκB which is S32A and S36A mutant form of IκB can continuously inhibit NF-κB because it is not phosphorylated by IκB Kinase and degraded by proteasome. Therefore, it has the great potential as treatment for various inflammatory diseases.

The present invention provides exosomes loaded with Super-repressor IκB protein and confirmed that Super-repressor IκB protein was delivered to the cytosol of the target cells. The results indicate that the exosome of the present invention loaded with Super-repressor IκB protein can be used for treatment of inflammatory diseases.

MyoD

MyoD is a protein that plays critical role in regulating muscle differentiation. MyoD belongs to a family of proteins known as myogenic regulatory factors (MRFs). MyoD is known to have binding interactions with hundreds of muscular gene promoters and to permit myoblast proliferation. Also, one of the main functions of MyoD is to remove cells from the cell cycle by enhancing the transcription of p21 and myogenin.

The exosomes comprising MyoD protein are prepared by expressing a fusion protein composed of an exosome specific marker and a target protein massively in the cell producing the exosomes at a high concentration. The exosomes loaded with MyoD can be used to treat myoblast-associated diseases.

Tbx18 (T-Box Transcription Factor 18)

Tbx18 codes for a member of an evolutionarily conserved family of transcription factors that plays a crucial role in embryonic development. Tbx18 is characterized by the presence of the DNA-binding T-box domain and it belongs to the vertebrate specific Tbx1 sub-family. Tbx18 acts as a transcriptional repressor by antagonizing transcriptional activators in the T-box family. Tbx18 is required in various developmental process in tissues and organs, including the heart and coronary vessels, the ureter and the vertebral column. It is also required for sinoatrial node (SAN) head area.

Tbx18 transduction is a method of turning on genes in heart muscle cells as a treatment for certain cardiac arrhythmias. In a healthy heart, sinoatrial nodal cells act as the heart's pacemaker and cause the heart to beat in a regular rhythm. The problem in sick sinus syndrome is that SA node is not functioning properly and is causing an irregular heartbeat. Expression of Tbx18 using adenovirus into atrial myocytes converts atrial muscle cells into SA node cells that initiate the heartbeat. Tbx18 can be a one of many forms of gene therapy that can cure cardiac arrhythmias.

The exosomes comprising Tbx18 protein are prepared by expressing a fusion protein composed of an exosome specific marker and a target protein massively in the cell producing the exosomes at a high concentration. The exosomes loaded with Tbx18 protein can be used for the treatment of sick sinus syndrome.

p53

Tumor protein p53 is known as the guardian of the genome because it conserves the stability of genome by preventing genome mutation. p53 can activate DNA repair proteins when DNA has sustained damage. In addition, p53 can arrest growth by holding the cell cycle at the G1/S regulation point on DNA damage recognition. Upon DNA damage and it is irreparable, p53 can induce apoptosis. Lastly, p53 is essential for the senescence response to short telomeres. p53 becomes activated in response to myriad stressors, including DNA damage, oxidative stress, osmotic shock, ribonucleotide depletion and deregulated oncogene expression.

If the p53 is damaged, tumor suppression is severely compromised. People who inherit only one functional copy of p53 gene will most likely develop tumors in early adulthood. Increasing the amount of p53 may seem a solution for treatment of tumors or prevention of their spreading.

The exosomes comprising p53 protein are prepared by expressing a fusion protein composed of an exosome specific marker and a target protein massively in the cell producing the exosomes at a high concentration. The exosomes loaded with p53 protein can be used for the treatment of varying types of cancers.

HMGB1 (High Mobility Group Box 1 Protein)

HMGB1 is among the most important chromatin proteins like histones. In the nucleus, HMGB1 interacts with nucleosomes, transcription factors and histones. This nuclear protein organizes the DNA and regulates transcriptions. After binding, HMGB1 bends DNA, which facilitates the binding of other proteins. It also interacts with nucleosomes to loosen packed DNA and remodel the chromatin.

HMGB1 is secreted by immune cells through leaderless secretory pathway. Activated macrophages and monocytes secrete HMGB1 as a cytokine mediator of inflammation. Antibodies that neutralize HMGB1 confer protection against damage and tissue injury during arthritis, colitis, ischemia, sepsis, etc.

The exosomes comprising HMGB1 protein are prepared by expressing a fusion protein composed of an exosome specific marker and a target protein massively in the cell producing the exosomes at a high concentration. The exosomes loaded with HMGB1 protein can be used for the treatment of inflammatory diseases.

NeuroD1

Neurogenic differentiation1, also called β2, is a transcription factor of the NeuroD-type. It mediates transcriptional activation by binding to E box-containing promoter consensus core sequences 5′-CANNTG-3′. It is contributed to the regulation of several cell differentiation pathways. It promotes the formation of early retinal ganglion cells, inner ear sensory neurons and granule cells forming either the cerebellum or the dentate gyrus cell layer of the hippocampus, endocrine islet cells of the pancreas and enteroendocrine cells of the small intestine.

The exosomes comprising NeuroD1 protein are prepared by expressing a fusion protein composed of an exosome specific marker and a target protein massively in the cell producing the exosomes at a high concentration. The exosomes loaded with NeuroD1 protein can be used for the regulation of neuron development.

Tumor-associated macrophages (TAMs) are a type of cell belonging to the macrophage lineage. They are found in close proximity or within tumor masses. TAMs are derived from circulating monocytes or resident tissue macrophages, which form the major leukocytic infiltrate found within the stroma of many tumor types. TAMs have been linked to poor prognosis in breast cancer, ovarian cancer, types of glioma and lymphoma; better prognosis in colon and stomach cancers and both poor and better prognoses in lung and prostate cancers.

TAMs are classified into two major phenotypes, M1 and M2. M1 TAMs suppress cancer progression, while M2 TAMs promote it. Several transcription factors are associated with the transition of M2 macrophage to M1 macrophage. The target proteins loaded in the exosomes in the present invention include transcription factors associated with the M2 to M1 conversion of macrophage. Examples of the target proteins are listed, but not limited to, in the following description.

IRF5

IRF5 is a member of the interferon regulatory factor, a group of transcription factor. It has role in virus-mediated activation of interferon and modulation of cell growth, differentiation, apoptosis and immune system activity. IRF5 work by directly interacting with DNA or with other proteins.

IRF5 acts as a molecular switch that controls whether macrophages will promote or inhibit inflammation. Blocking the production of IRF in macrophage can help treat a wide range of autoimmune disease and upregulating IRF5 levels can help treat people whose immune system are weak or damaged.

The exosomes comprising IRF5 protein are prepared by expressing a fusion protein composed of an exosome specific marker and a target protein massively in the cell producing the exosomes at a high concentration. The exosomes loaded with IRF5 protein can be used for macrophage transition from M2 to M1 for the treatment of varying types of cancers.

IRF3

IRF3 is a member of the interferon regulatory factors, a group of transcription factor. IRF3 includes functional domains, nuclear export signal, a DNA-binding domain, a C-terminal IRF association domain and several regulatory sites. It is found in an inactive form in the cytoplasm of uninfected cells. Upon viral infection, double stranded RNA or toll-like receptor signaling, it is phosphorylated by IKBKE and TBK1 kinases. This leads to dimerization and nuclear localization. IRF3 can activate distinct gene expression programs in macrophages.

The exosomes comprising IRF3 protein are prepared by expressing a fusion protein composed of an exosome specific marker and a target protein massively in the cell producing the exosomes at a high concentration. The exosomes loaded with IRF3 protein can be used for macrophage transition from M2 to M1 for the treatment of varying types of cancers.

STAT1

Signal transducer and activator of transcription 1 is a transcription factor, member of the STAT protein family. STAT1 can be activated by several ligands such as interferon alpha, interferon gamma, epidermal growth factor, platelet derived growth factor or interleukin 6.

Following type I IFN binding to cell surface receptors, JAK gets activated and phosphorylates STAT1 and STAT2. STATs dimerize and associate with ISGF3G/IRF-9 to form a complex termed ISGF3 transcription factor. ISGF3 binds to the IFN stimulated response element to activate the transcription of IFN-stimulated genes.

In response to type II IFN, STAT1 is tyrosine and serine phosphorylates. It forms a homodimer and binds to IFN gamma activated sequence to drive the expression of target genes, inducing a cellular antiviral state.

The exosomes comprising STAT1 protein are prepared by expressing a fusion protein composed of an exosome specific marker and a target protein massively in the cell producing the exosomes at a high concentration. The exosomes loaded with STAT1 protein can be used for the treatment of varying types of cancers.

SOCS3

Suppression of cytokine signaling is a member of STAT-induced STAT inhibitor. STAT-induced STAT inhibitors are cytokine-inducible negative regulators of cytokine signaling. SOCS3 is induced by various cytokines like IL6, IL10, and IFN-gamma.

Overexpression of SOCS3 inhibits insulin signaling in adipose tissue and liver but not in muscle. But deletion of SOCS3 in the skeletal muscle of mice protects against the obesity. SOCS3 also contributes to both leptin resistance and insulin resistance as a result of increased ceramide synthesis. Study shows that removal of the SOCS gene prevents against insulin resistance in obesity. SOCS3 protein can bind to JAK2 and inhibits the activity of JAK2.

The exosomes comprising SOCS3 protein are prepared by expressing a fusion protein composed of an exosome specific marker and a target protein massively in the cell producing the exosomes at a high concentration. The exosomes loaded with SOCS3 protein can be used for the treatment of varying types of cancers.

<Antibodies>

Antibodies are the proteins that recognize and bind to their specific antigen via the Fab's variable region on the tip of the “Y”-shaped antibody. Antibodies can suppress the activity of the target antigen proteins by binding to them.

The target proteins loaded in the exosomes in the present invention include antibodies and antibody-associated peptides. Examples of the target proteins are listed, but not limited to, in the following description.

Antibodies and Associated Peptides

pYSTAT3 Intrabody

STATs (Signal Transducer and Transcriptions) are transcription factors that have been identified: STAT1, STAT2, STAT3, STAT4, STAT5 (STAT5A and STAT5B), and STAT6. STAT3 proteins have C-terminal transactivation domain (Tyrosine 705 and Serine 727 residues for the major phosphorylation sites of STAT3). Tyrosine phosphorylation and subsequent dimerization of STAT3 promote the transportation to the nucleus and transcriptional activation.

The JAK/STAT3 signaling pathway is identified in growth factor-induced activation of interferon signaling and involved in proliferation, differentiation, apoptosis, angiogenesis, oncogenesis and immunity. Therefore, STAT3 proteins can be a good target as a single agent or combination therapeutics for development of anticancer drugs.

The exosomes of the present invention loaded with pYSTAT3 intrabody was prepared and confirmed that pYSTAT3 intrabody was delivered to the cytosol of the target cells. The results indicates that the exosome of the present invention loaded with pYSTAT3 intrabody can be used for treatment of cancer.

<Others>

Apoptosis-Associated Proteins

Apoptosis (programmed cell death) is the process for eliminating damaged cells by various factors and abnormal apoptosis induces tumorigenicity. Based on this reason, researches about inducing apoptosis of tumor have actively progressed as tumor-eliminating strategy. Condensation of chromatin by cell atrophy, apoptotic body formation, and DNA fragmentation are the features of apoptosis. The apoptosis is induced by two different routes; one is the intrinsic pathway through mitochondria, and the other is the extrinsic pathway through death receptors. The apoptosis is regulated variously, for example activation of pro-apoptotic Bcl-2 family, segmentation of pro-caspase, and fragmentation of poly ADP-ribose polymerase (PARP), and so on. Especially caspases belonged to cysteine proteases are being pro-enzyme in normally proliferated cells and activated by apoptotic inducing signals, then has the significant role in apoptosis through involving cargo proteins such as PARP.

Most apoptotic stimuli induce the apoptosis of mammalian cells through the pathway controlled by members of Bcl-2 gene family which are coding homologous protein group including agonist and antagonist of apoptosis such as Bcl-2, and Bcl-xL. These members share the sequence homologous domain even though they are regulated discriminately. During apoptosis, the anti-apoptotic or pro-apoptotic effect of Bcl-2 and Bax (21% identity with Bcl-2 at the protein level) is regulated by homo- and heterodimers, which are differently formed by the ratio of Bcl-2 to Bax.

Pro-Apoptotic Proteins: Bax

Bax (Bcl-2-associated X protein) is the one of Bcl-2 protein family, so-called Bcl-2 like protein 4. Aforementioned Bax, which binds to the external membrane of mitochondria and its 4 residues of C-terminal protrude on intermembrane space of mitochondria, has the role to activate apoptosis. Specific information about aforementioned protein and base sequence of its gene is noticed on NCBI (GenBank: NM_001291428, NP_001278357, etc.).

Bax is the one of Bcl-2 gene family synthesizing pro-apoptotic protein. Bax is inhibited its transcription by mutant p53. It has well known that insertion or deletion of Bax base sequence is the cause of markedly decreased expression of Bax in cell lines of blood, colon, and rectal cancer.

It is known that Bax involves in apoptosis of neuron in development, homeostatic equilibrium of lymphatic and genital system, cell death by DNA damage, damage of ischemia reperfusion and so on.

The present invention provides exosomes loaded with Bax protein and confirmed that Bax protein was delivered to the cytosol of the target cells. The results indicates that the exosome of the present invention loaded with Bax protein can be used for treatment of cancer.

Anti-Apoptotic Proteins: Bcl-xL

B-cell lymphoma-extra-large (Bcl-xL), encoded by the BCL2-like 1 gene, is a transmembrane molecule in the mitochondria. It is a member of the Bcl-2 family of proteins, and acts as an anti-apoptotic protein by preventing the release of mitochondrial contents such as cytochrome c, which leads to caspase activation and ultimately, programmed cell death.

It is a well-established concept in the field of apoptosis that relative amounts of pro- and anti-survival Bcl-2 family of proteins determine whether the cell will undergo cell death; if more Bcl-xL is present, then pores are non-permeable to pro-apoptotic molecules and the cell survives. Similar to Bcl-2, Bcl-xL has been implicated in the survival of cancer cells by inhibiting the function of p53, a tumor suppressor.

The exosomes comprising Bcl-xL protein are prepared by expressing a fusion protein composed of an exosome specific marker and a target protein massively in the cell producing the exosomes at a high concentration. The exosomes loaded with Bcl-xL protein can be used for the regulation of apoptosis.

Etc.

Multifunctional signal molecules: AIMP (Aminoacyl-tRNA synthase-interacting multifunctional proteins)

Aminoacyl tRNA synthase complex-interacting multifunctional protein 1 (AIMP1) is a non-catalytic component of the multi-synthase complex. Stimulates the catalytic activity of cytoplasmic arginyl-tRNA synthase. Possesses inflammatory cytokine activity. Negatively regulates TGF-beta signaling through stabilization of SMURF2 by binding to SMURF2 and inhibiting its SMAD7-mediated degradation. Involved in glucose homeostasis through induction of glucagon secretion at low glucose levels. Promotes dermal fibroblast proliferation and wound repair.

Plays a role in angiogenesis by inducing endothelial cell migration at low concentrations and endothelial cell apoptosis at high concentrations. Induces maturation of dendritic cells and monocyte cell adhesion. Modulates endothelial cell responses by degrading HIF-1A through interaction with PSMA7.

Aminoacyl tRNA synthase complex-interacting multifunctional protein 2 (AIMP2) is required for assembly and stability of the aminoacyl-tRNA synthase complex. Mediates ubiquitination and degradation of FUBP1, a transcriptional activator of MYC, leading to MYC down-regulation which is required for alveolar type II cell differentiation. Accumulates in brains affected by autosomal-recessive juvenile Parkinsonism, idiopathic Parkinson disease and diffuse Lewy body disease.

The exosomes comprising AIMP1 and AIMP2 proteins are prepared by expressing a fusion protein composed of an exosome specific marker and a target protein massively in the cell producing the exosomes at a high concentration. The exosomes loaded with AIMP1 or AIMP2 protein can be used for multifunctional regulation.

Fluorescent Proteins (mCherry, GFP)

Fluorescent proteins are members of a structurally homologous class of proteins that share the unique property of being self-sufficient to form a visible wavelength chromophore from a sequence of 3 amino acids within their own polypeptide sequence. It is common research practice for biologists to introduce a gene (or a gene chimera) encoding an engineered fluorescent protein into living cells and subsequently visualize the location and dynamics of the gene product using fluorescence microscopy.

The most popular applications of fluorescent proteins involve exploiting them for imaging of the localization and dynamics of specific organelles or recombinant proteins in live cells. For imaging of a specific organelle, standard molecular biology techniques are used to fuse the gene encoding the fluorescent protein to a cDNA encoding a protein or peptide known to localize to that specific organelle. This fusion is done such that the chimeric gene will be expressed as a single polypeptide, creating a covalent link between the targeting motif and the fluorescent protein. A plasmid containing the chimeric gene under control of a suitable promoter is used to transfect mammalian cells that then express the gene to produce the corresponding chimeric protein. The chimera localizes to the target organelle and thus renders it fluorescent. Through the use of fluorescence microscopy, the morphology, dynamics, and distribution of the organelle can be imaged as a function of time.

mCherry is a monomeric fluorescent construct with peak excitation/emission at 587 nm/610 nm, respectively. It is resistant to photobleaching and is stable. It matures quickly, with a t0.5 of 15 minutes, allowing it to be visualized soon after translation.

The green fluorescent protein (GFP) is a protein composed of 238 amino acid residues (26.9 kDa) that exhibits bright green fluorescence when exposed to light in the blue to ultraviolet range. Although many other marine organisms have similar green fluorescent proteins, GFP traditionally refers to the protein first isolated from the jellyfish Aequorea Victoria. The GFP from A. Victoria has a major excitation peak at a wavelength of 395 nm and a minor one at 475 nm. Its emission peak is at 509 nm, which is in the lower green portion of the visible spectrum.

The present invention prepared exosomes loaded with mCherry or GFP protein and confirmed that mCherry or GFP protein was delivered to the cytosol of the target cells. The results indicate that the exosome of the present invention loaded with mCherry or GFP protein can be used for imaging of the localization and dynamics of exosomes and linked proteins in live cells or animal.

Nucleic Acid-Binding Proteins (Ex. RNPs)

Nucleoproteins are any proteins that are structurally associated with nucleic acids, either DNA or RNA. A deoxyribonucleoprotein (DNP) is a complex of DNA and protein. The prototypical examples are nucleosomes, complexes in which genomic DNA is wrapped around clusters of eight histone proteins in eukaryotic cell nuclei to form chromatin. Protamines replace histones during spermatogenesis. DNPs in this kind of complex interact to generate a multiprotein regulatory complex in which the intervening DNA is looped or wound. The DNPs participate in regulating DNA replication and transcription.

A ribonucleoprotein (RNP) is a complex of RNA and protein. The enzyme telomerase, vault ribonucleoproteins, RNase P, hnRNP and small nuclear RNPs (snRNPs), and ribosomes are ribonucleoproteins. The RNPs play a role of protection. mRNAs never occur as free RNA molecules in the cell. They always associate with ribonucleoproteins and function as ribonucleoprotein complexes.

The exosomes comprising DNPs or RNPs are prepared by expressing a fusion protein composed of an exosome specific marker and a target protein massively in the cell producing the exosomes at a high concentration. The exosomes loaded with DNPs or RNPs can be used for genetic regulations or nucleic acid transportable exosomes.

The present invention confirmed that the cargo protein was successfully delivered to cytosol of a target cell by using the exosome containing the cargo protein therein, and thereby the present invention provides a method to treat disease using exosome by regulating intracellular signaling efficiently in cytosol.

Another object of the present invention is to provide a pharmaceutical composition for preventing or treating inflammatory diseases containing the exosome as an active ingredient.

Another object of the present invention is to provide a pharmaceutical composition for preventing or treating cancer containing the exosome as an active ingredient.

Another object of the present invention is to provide a pharmaceutical composition for preventing or treating oxygen-related diseases containing the exosome as an active ingredient.

Another object of the present invention is to provide a composition for producing a conditional knockout allele of a target gene containing the exosome as an active ingredient.

Another object of the present invention is to provide a DNA sequence manipulating composition containing the exosome as an active ingredient.

Another object of the present invention is to provide a pharmaceutical composition for preventing or treating Gaucher's disease containing the exosome as an active ingredient.

To develop an efficient method for preparing exosome containing a cargo protein, the present inventors studied with various attempts. In the course of our study, the inventors paid attention on exosome specific markers (CD9, CD63, CD81, and CD82). These markers belong to the tetraspanin family and are commonly 4-times penetration type membrane proteins. The present inventors predicted that when a cargo protein was conjugated on the membrane protein of exosome, the cargo protein would be relatively easily included in the inside of exosome.

By expressing the fusion protein composed of an exosome specific marker which is rich especially on exosome membrane and can penetrate cell membrane and a cargo protein in the cell producing exosome endogenously at a high concentration, exosome containing a cargo protein can be massively produced.

Particularly, the method for preparing exosome comprising a cargo protein of the present invention is characterized by introduction of polynucleotide encoding the fusion protein composed of an exosome specific marker and a cargo protein in the cell producing exosome.

At this time, in the prepared exosome, a cargo protein is fused with an exosome specific marker embedded in exosome membrane.

The said cargo protein is bound to the membrane protein of exosome and is not separated even after it arrives at the target cell. To solve this problem, various attempts have been made. As a result, a technique has been developed for the preparation of exosome comprising a cargo protein by conjugating a cargo protein temporarily to a marker protein. For example, a photo-specific binding protein such as CIBN and CRY2 can be used herein. Particularly, CIBN is expressed in a form fused with CD9, which is one of the marker proteins. In the meantime, a gene encoding the fusion protein of CRY2 and a cargo protein is introduced in the cell producing exosome. The CIBN-CD9 fusion protein expressed in the exosome production cell can be included due to CD9. At this time, when the cell is irradiated with blue LED light, CRY2 domain of the cargo protein-CRY2 fusion protein expressed in the exosome production cell is bound to the CD9 fused CIBN domain. As a result, the reversible ‘cargo protein-CRY2-CIBN-CD9 fusion protein’ is produced. This fusion protein can be included in the inside of exosome due to CD9. Once exosome containing a cargo protein therein is produced and the irradiation with the blue LED light is terminated, CIBN-CRY2 link is broken and thereby the cargo protein remains in exosome as being apart from the cell membrane of exosome, resulting in the preparation of exosome comprising the cargo protein (FIGS. 5˜10).

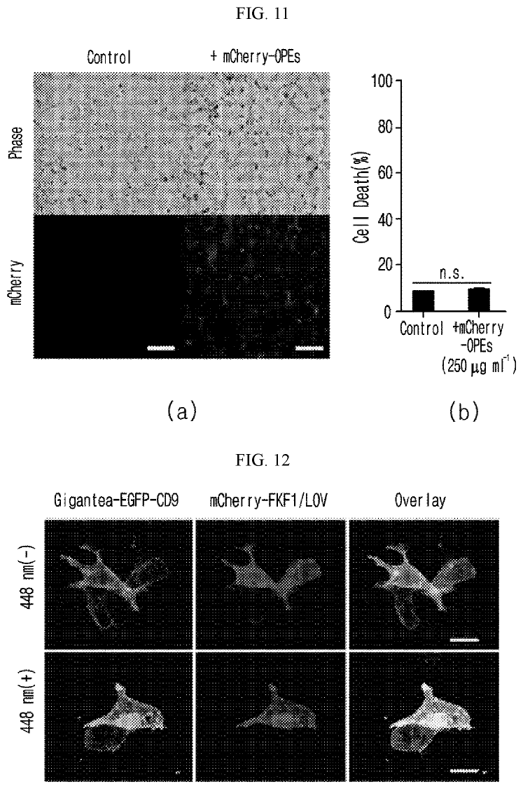

This kind of exosome prepared by the method of the present invention is completely different in its effect from the conventional exosome containing a target material. The conventional exosome is expressed as being fused onto an exosome specific marker in order to present a cargo protein inside of the exosome, so that the cargo protein, even though it is included in the inside of the exosome, it is not free and instead presented as being attached on the membrane of exosome, suggesting that the cargo protein cannot be separated from the membrane of exosome and therefore it can be delivered into a target cell only when the exosome is fused on the cell wall of the target cell. Moreover, even after the fusion onto the target cell, the cargo protein remains as being conjugated to the membrane of exosome. Therefore, the probability that the cargo protein exhibits its effect in the target cell is very low. However, the exosome of the present invention presents a cargo protein which resides as free and not being conjugated on the membrane of exosome. So, when such exosome enters cytosol by endocytosis of the target cell, it does not adhere to the membrane of exosome, and when the exosome is decomposed therein, the included cargo protein can be delivered in cytosol and is free to move in cytosol of the target cell, suggesting that the cargo protein is fully active with its physiological activity in the target cell cytosol (FIG. 11).

The binding level of the cargo protein to the marker protein can be changed according to the intensity of the light to be irradiated. Therefore, by regulating the intensity of the light, the concentration of the cargo protein collected in exosome can be controlled.

The method for preparing exosome containing a cargo protein by using a photo-specific binding protein has not been reported yet and was proposed first by the present inventors.

Particularly, the method for preparing exosome containing a cargo protein of the present invention is composed of the following steps: (a) introducing the polynucleopeptide encoding the fusion protein (fusion protein I) composed of an exosome specific marker and the first photo-specific binding protein and the polynucleotide encoding the fusion protein (fusion protein II) composed of a cargo protein and the second photo-specific binding protein that can be linked to the first photo-specific binding protein in the exosome production cell; (b) irradiating the exosome production cell with light that can cause the conjugation between the first photo-specific binding protein and the second photo-specific binding protein; and (c) terminating the irradiation after the production of exosome finished in the exosome production cell.

The term “exosome” in the present invention indicates a small vesicle with the plasma membrane structure, which is originated from an intracellular specific compartment called multi-vesicular bodies (MVBs) and is released or secreted from the cell.

In this invention, exosome plays a role as a carrier to deliver a cargo protein into a target cell or tissue by carrying the cargo protein in itself. At this time, the cargo protein carried by the exosome works for the target cell or tissue to help the treatment or diagnosis of a specific disease.

The term “exosome production cell” in this invention indicates the cell that is able to produce exosome.

In this invention, the exosome production cell is not limited but is preferably exemplified by B-lymphocyte, T-lymphocyte, dendritic cell, megakaryocyte, macrophage, stem cell, and tumor cell, etc. For example, in this invention, HEK293T cell that is a kind of immortalized cell line was used as the exosome production cell.

The term “exosome specific marker” in this invention indicates a protein which is rich on the membrane of exosome.

In this invention, the exosome specific marker is not limited but is preferably exemplified by CD9, CD63, CD81, and CD82, etc. For example, in a preferred embodiment of the present invention, CD9 was used as the exosome specific marker. CD9, CD63, CD81, and CD82 are 4-times penetration type membrane proteins that allow the cargo protein to be easily present in exosome when the cargo protein is bound to the membrane protein of the exosome.

The term “photo-specific binding protein” in this invention is also called photo-induced heterodimer formation protein or photo-induced homodimer formation protein, which indicates a protein that is able to form a heterodimer by combining with different proteins or to form a homodimer by combining with another protein in the same kind when the light of a specific wavelength is irradiated.

In this invention, the photo-specific binding protein is not limited but is preferably exemplified by the photo-induced heterodimer formation protein or CIB (cryptochrome-interacting basic-helix-loop-helix protein), CIBN (N-terminal domain of CIB), PhyB (phytochrome B), PIF (phytochrome interacting factor), FKF1 (Flavinbinding, Kelch repeat, F-box 1), GIGANTEA, CRY (cryptochrome), and PHR (phytolyase homologous region), etc.

In particular, when the photo-specific binding protein is the photo-induced heterodimer formation protein, two types of photo-specific binding protein (the first and the second photo-specific binding proteins) can be used. When the first photo-specific binding protein is CIB or CIBN, the second photo-specific binding protein can be CRY or PHR. When the first photo-specific binding protein is PhyB, the second photo-specific binding protein can be PIF. When the first photo-specific binding protein is GIGANTEA, the second photo-specific binding protein can be FKF1.

For example, in a preferred embodiment of the present invention, CIBN was used as the first photo-specific binding protein, and CRY2 was used as the second photo-specific binding protein. The wavelength of the light used herein was the blue light with 460˜490 nm. The intensity of the light was 20˜50 W.

In the meantime, in order to confirm the expression and to find out the location of the first fusion protein composed of the exosome specific marker and the first photo-specific binding protein expressed therein, a marker protein can be fused thereto. For example, in a preferred embodiment of the invention, the fluorescent protein EGFP was inserted in the first fusion protein wherein CIBN and CD9 or GIGANTEA and CD are linked together. So, the expression pattern (expression and expression level) and the intracellular location of the first fusion protein can be investigated by the expression of the first fusion protein as harboring the fluorescent protein EGFP.

The term “cargo protein” in this invention indicates a protein which is expressed as a fusion protein conjugated with the second photo-specific binding protein to locate the cargo protein inside the exosome.

In this invention, the cargo protein can be carried by exosome after being expressed in cells. The cargo protein is not limited but is preferably a disease treating protein or disease diagnosing protein. For example, in a preferred embodiment of the present invention, mCherry with fluorescence was used as the cargo protein.

An example of the cargo proteins in the present invention is selected from, but not limited to, Matrix metalloproteinases (MMPs) proteins, Tissue inhibitor of metalloproteinases (TIMPs) proteins, caspases proteins, caspases inhibitory proteins, cathepsins proteins or cathepsin inhibitory proteins,
wherein,
MMPs proteins are such as, but not limited to, MMP1 protein (SEQ ID NO: 13);
TIMPs proteins are such as, but not limited to, TIMP1 protein (SEQ ID NO: 14), TIMP2 protein (SEQ ID NO: 15), TIMP3 protein (SEQ ID NO: 16), or TIMP4 protein (SEQ ID NO: 17);
caspases proteins are such as, but not limited to, casepase 1 protein (SEQ ID NO: 18), casepase 2 protein (SEQ ID NO: 19), casepase 3 protein (SEQ ID NO: 20), casepase 4 protein (SEQ ID NO: 21), casepase 5 protein (SEQ ID NO: 22), casepase 6 protein (SEQ ID NO: 23), casepase 7 protein (SEQ ID NO: 24), casepase 8 protein (SEQ ID NO: 25), casepase 9 protein (SEQ ID NO: 26), casepase 10 protein (SEQ ID NO: 27), casepase 11 protein (SEQ ID NO: 28), casepase 12 protein (SEQ ID NO: 29), casepase 13 protein (SEQ ID NO: 30), or casepase 14 protein (SEQ ID NO: 31);
caspases inhibitory proteins are such as, but not limited to, proteins inhibiting caspase proteins represented by SEQ ID NO: 18-31 or any proteins inhibiting caspase;
cathepsins proteins are such as, but not limited to, cathepsins A protein (SEQ ID NO: 32), cathepsins B protein (SEQ ID NO: 33), cathepsins C protein (SEQ ID NO: 34), cathepsins D protein (SEQ ID NO: 35), cathepsins E protein (SEQ ID NO: 36), cathepsins F protein (SEQ ID NO: 37), cathepsins G protein (SEQ ID NO: 38), cathepsins H protein (SEQ ID NO: 39), cathepsins K protein (SEQ ID NO: 40), cathepsins L1 protein (SEQ ID NO: 41), cathepsins L2 protein (SEQ ID NO: 42), cathepsins O protein (SEQ ID NO: 43), cathepsins S protein (SEQ ID NO: 44), cathepsins W protein (SEQ ID NO: 45), or cathepsins Z protein (SEQ ID NO: 46); and
cathepsin inhibitory proteins are such as, but not limited to, proteins inhibiting cathepsin proteins represented by SEQ ID NO: 32-46 or any protein inhibiting cathepsins.

Another example of the cargo proteins in the present invention is selected from, but not limited to, Cre recombinase, Cas protein, Caspase-activated DNase (CAD) proteins, β-glucocerebrosidase (GBA), p38 mitogen-activated protein kinases, Phosphatase and tensin homolog (PTEN), Janus kinase (JAK), ubiquitin ligase, luciferase, peroxiredoxin (Prx) I or II, protein inhibiting NF-κB, MyoD proteins, Tbx18 proteins, p53 proteins, High mobility group box 1 (HMGB1) proteins, neurogenic differentiation1 (Neuro-D1) proteins, Interferon regulatory factor 5 (IRF5) proteins, Interferon regulatory factor 3 (IRF3) proteins, Signal transducer and activator of transcription 1 (STAT1) proteins, Suppressor of cytokine signaling 3 (SOCS3) proteins, Signal transducer and activator of transcription 2 (STAT2) proteins, proteins inhibiting phosphorylated STAT3 (pYSTAT3), Bax (Bcl2-associated X protein), B-cell lymphoma-extra-large (Bcl-xX) proteins, Aminoacyl-tRNA synthase-interacting multifunctional proteins (AIMPs), mCherry proteins, green fluorescent proteins (GFP), or nucleoproteins binding to nucleic acid,
wherein,
Cre recombinase recombines the DNA between loxP sites by recognizing them in DNA and includes, but not limited, Cre recombinase represented by SEQ ID NO: 9;
Cas protein has endonuclease or nickase activity when it combines the complex with guide RNA. In some embodiment, Cas protein is Cas9 protein such as Cas protein represented by SEQ ID NO: 10, its mutant, or Cpf1 protein such as amino acids represented by SEQ ID NO: 11;
CAD protein is such as the amino acids represented by SEQ ID NO: 47;
β-glucocerebrosidase (GBA) is such as the amino acids represented by SEQ ID NO:12;
p38 mitogen-activated protein kinases (p38 MAPKs) proteins are such as p38-α or its mutants and include amino acids represented by SEQ ID NOs: 48-51;
Inhibitor kappa B kinase (IKK) proteins are such as the amino acids represented by SEQ ID NO: 83;
Nuclear factor-kappa B (NF-κB) proteins are such as the amino acids represented by SEQ ID NO: 84;
Phosphatase and tensin homolog (PTEN) proteins are such as the amino acids represented by SEQ ID NO: 52;
Janus kinase (JAK) proteins include JAK1, JAK2, JAK3 and TYK2, wherein JAK1 proteins are such as the amino acids represented by SEQ ID NO: 53, JAK2 proteins are such as the amino acids represented by SEQ ID NO: 54, JAK3 proteins are such as the amino acids represented by SEQ ID NO: 55, and TYK2 proteins are such as the amino acids represented by SEQ ID NO: 56; ubiquitin ligase proteins include c-CBL, PRKN, RBX1, TRAF2 and Mdm2, wherein ubiquitin ligase proteins are such as the amino acids represented by SEQ ID NO: 57 to 61;
luciferase proteins are such as the amino acids represented by SEQ ID NO: 62;
peroxiredoxin (Prx) I or II has the effect of inhibiting cytotoxicity from oxidative stress, wherein peroxiredoxin I is such as the amino acids represented by SEQ ID NO: 7, and peroxiredoxin II is the amino acids represented by SEQ ID NO: 8;
protein inhibiting NF-κB is super-repressor-IκB which inactivates NF-κB by binding with it in cytoplasm, wherein the super-repressor-IκB protein, which is S32A and S36A mutant form of IκB, is not phosphorylated by IκB Kinase (IKK) and consequently it can continuously inhibit NF-κB, and NF-κB inhibiting proteins are such as the amino acids represented by one of SEQ ID NO: 1 to 5, exemplified by IκB-α, IκB-β, IκB-ε, BCL-3 or their mutant;
MyoD proteins are such as the amino acids represented by SEQ ID NO: 63;
Tbx18 proteins are such as the amino acids represented by SEQ ID NO: 64;
p53 proteins are such as the amino acids represented by SEQ ID NO: 65;
High mobility group box 1 protein (HMGB1) proteins are such as the amino acids represented by SEQ ID NO: 66;
Neurogenic differentiation1 (Neuro-D1) proteins are such as the amino acids represented by SEQ ID NO: 67;
Interferon regulatory factor 5 (IRF5) proteins are such as the amino acids represented by SEQ ID NO: 68;
Interferon regulatory factor 3 (IRF3) proteins are such as the amino acids represented by SEQ ID NO: 69;
Signal transducer and activator of transcription 1 (STAT1) proteins are such as the amino acids represented by SEQ ID NO: 70;
Suppressor of cytokine signaling 3 (SOCS3) proteins are such as the amino acids represented by SEQ ID NO: 71;
Signal transducer and activator of transcription 2 (STAT2) proteins are such as the amino acids represented by SEQ ID NO: 72;
proteins inhibiting phosphorylated STAT3 (pySTAT3) including pySTAT3 intrabody antibody proteins, which binds to pySTAT3 to deactivate pySTAT3, and are such as the amino acids represented by SEQ ID NO: 73 or any proteins inhibiting pySTAT3;
Bax (Bcl2-associated X protein) is such as the amino acids represented by SEQ ID NO: 6;
B-cell lymphoma-extra-large (Bcl-xL) proteins are such as the amino acids represented by SEQ ID NO: 74;
Aminoacyl-tRNA synthase-interacting multifunctional proteins (AIMPs) include AIMP1 and AIMP2, wherein AIMP1 proteins are such as the amino acids represented by SEQ ID NO: 75 and AIMP2 proteins are such as the amino acid represented by SEQ ID NO: 76;
mCherry proteins are such as the amino acids represented by SEQ ID NO: 77;
green fluorescent protein (GFP) are such as the amino acids represented by SEQ ID NO: 78;
nucleoproteins binding to nucleic acids include deoxyribonucleoprotein (DNP) binding to DNA or ribonucleoprotein (RNP) binding to RNA, wherein DNP includes RBBP4 or NAP1L4 and RNP include Telomerase, Heterogenous nuclear ribonucleoprotein K (HNRNPK) and wherein nucleoproteines are such as the amino acids represented by SEQ ID NOs: 79-82, nucleosome, protamine, small nuclear RNPs (snRNPs) or mutants thereof, or any proteins binding to nucleic acid.

The term “culture” in this invention indicates a method to grow cells or microorganisms in a properly controlled environment.

In this invention, a transformant was cultured for 1˜3 days and then the medium was replaced with a serum-free medium, followed by further culture for 2˜5 days.

In this invention, the method for culturing the transformant is any of those well known to those in the art.

The said medium herein indicates a notified medium widely used for animal cell culture, which can be selected from the group consisting of commercially available serum-free media, protein-free media, and chemically defined media.

The serum-free media above are used for animal cell culture, which are free from bovine serum and are exemplified by SFM4CHO (HyClone) and EX-Cell (JHR Bioscience). Insulin like growth factor I (IGF-I), ethanolamine, ferric chloride, and phosphatidyl choline can be added to the media, but not always limited thereto.

The protein-free media above are animal cell culture media, from which animal originated proteins especially high molecular proteins in particular having the molecular weight of at least 10 kDa are eliminated. The protein-free media can be ProCHO (Lonza) and PF-CHO (HyClone), but not always limited thereto.

The chemically defined media above are animal cell culture media which do not include any animal originated components and instead have components all having defined chemical structures. The chemical defined media can be CDM4CHO (HyClone), PowerCHO2CD (Lonza), and CD-optiCHO (Life Technologies), but not always limited thereto.

The term “the first fusion protein” in this invention indicates the fusion protein made by binding between the exosome specific marker and the first photo-specific binding protein.

In this invention, the order of arrangement of the exosome specific marker and the first photo-specific binding protein contained in the first fusion protein is not limited as long as the first photo-specific binding protein is located in the direction toward the inside of exosome when the first fusion protein is expressed in the exosome production cell. For example, N-terminal of the first photo-specific binding protein can be conjugated to C-terminal of the exosome specific marker.

The exosome specific marker and the first photo-specific binding protein which compose the first fusion protein are linked directly each other or can be connected by a linker. The linker above is not limited as long as the first fusion protein is expressed in the exosome production cell with presenting the first photo-specific binding protein located in the direction toward the inside of exosome, but is preferably a peptide linker composed of amino acids and more preferably a flexible peptide linker. The peptide linker can be expressed by using an expression vector wherein the nucleic acids encoding the linker are connected with other nucleic acids encoding each domain in frame.

The term “the second fusion protein” indicates a fusion protein in which the second photo-specific binding protein and the cargo protein are combined.

In this invention, the order of arrangement of the second photo-specific binding protein and the cargo protein contained in the second fusion protein is not limited as long as the second fusion protein is located inside of exosome as being conjugated with the first photo-specific binding protein region of the first fusion protein in the exosome production cell. For example, N-terminal of the cargo protein can be conjugated to C-terminal of the second photo-specific binding protein.

The second photo-specific binding protein and the cargo protein which compose the second fusion protein are linked directly each other or can be connected by a linker. The linker above is not limited as long as the second fusion protein is located inside of exosome as being conjugated with the first photo-specific binding protein of the first fusion protein in the exosome production cell, but is preferably a peptide linker composed of amino acids and more preferably a flexible peptide linker. The peptide linker can be expressed by using an expression vector wherein the nucleic acids encoding the linker are connected with other nucleic acids encoding each domain in frame.

In addition, each fusion protein above can include a polypeptide having the sequence wherein at least one amino acid residues are different from those in the wild type amino acid sequence of each domain included therein. Amino acid exchange in proteins and polypeptides without changing the overall activity of a molecule is well known to those in the art. The most common exchange occurs between Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, and Asp/Gly. In addition, a protein having increased structural stability against heat or pH or increased protein activity due to mutation or modification of amino acid sequence can be included.

Lastly, the fusion protein above or the polypeptide of each domain comprising the fusion protein can be prepared by the chemical peptide synthesis method well informed to those in the art, or prepared by the following method. A gene encoding each domain is amplified by PCR (polymerase chain reaction) or synthesized by the conventional method well known to those in the art. The gene is cloned in an expression vector and expressed.

In the meantime, each fusion protein can be expressed in the exosome production cell by introducing a polynucleotide encoding each fusion protein in the exosome production cell. At this time, the polynucleotide is introduced in the exosome production cell by the conventional method well informed to those in the art. For example, an expression vector can be used for the

### INTRODUCTION

The term “expression vector” in this invention is a recombinant vector capable of expressing a target peptide in host cells. This vector indicates a gene construct containing essential regulators operably linked so as to express the gene insert. The expression vector includes expression control elements such as a start codon, a termination codon, a promoter, and an operator. The start codon and termination codon are generally understood as a part of the nucleotide sequence encoding a polypeptide. They are supposed to be working when a gene construct is introduced and to reside in a coding sequence in frame. The promoter of the vector can be constitutive or inductive.

The term “operably linked” in this invention indicates the status when the nucleic acid expression regulation sequence functioning as usual and the nucleic acid sequence encoding a cargo protein or RNA are linked by functional linkage. For example, a promoter is operably linked to a nucleic acid sequence encoding a protein or RNA in order to affect the expression of the coding sequence. The functional linkage with an expression vector can be achieved by the recombinant DNA technology well known to those in the art, and particularly the site-specific DNA cleavage and linkage can be achieved by using the conventional enzyme well known to those in the art.

The said expression vector can include a signal sequence for the discharge of a fusion polypeptide in order to promote the separation of a protein from the cell culture medium. A specific initiation signal might be necessary for the efficient translation of the inserted nucleic acid sequence. These signals contain ATG start codon and its neighboring sequences. In some cases, an exogenous translational control signal, which may include the ATG start codon, should be provided. These exogenous translational control signals and start codon can be various natural and synthetic sources. The expression efficiency can be increased by the introduction of appropriate transcription or translation enhancers.

In a preferred embodiment of the present invention, the expression vector is able to express a cargo protein conjugated with a tag in order to confirm the insertion of a cargo protein inside the exosome. The tag herein is to confirm the presence of a cargo protein, which can be conjugated to the region opposite to the region of the second photo-specific binding protein conjugation. For example, a fluorescent protein such as a red fluorescent protein and a green fluorescent protein is used as a tag to be conjugated to C-terminal of a cargo protein.

The cargo protein prepared as described above is expressed in the exosome production cell. Once exosome is produced, it is investigated whether or not the fluorescent protein tag is detected, by which the presence of the cargo protein in exosome can be confirmed.

The term “light” in this invention indicates the light to be irradiated in order to combine temporarily the first photo-specific binding protein and the second photo-specific binding protein expressed in the exosome production cell.

As described hereinbefore, the first photo-specific binding protein is expressed as the first fusion protein conjugated with the exosome specific marker, while the second photo-specific binding protein is expressed as the second fusion protein conjugated with the cargo protein. When the light is irradiated to the exosome production cell, the first photo-specific binding protein is combined with the second photo-specific binding protein, and as a result the fusion protein complex comprising the exosome specific marker—the first photo-specific binding protein—the second photo-specific binding protein—the cargo protein is formed temporarily. When exosome is produced in the exosome production cell, the cargo protein can be linked to the exosome due to the exosome specific marker. At this time, the cargo protein presents inside the exosome and when the irradiation with the light is stopped after the production of the exosome, the first photo-specific binding protein is separated from the second photo-specific binding protein and thereby the cargo protein included in the exosome is to be discharged together with the exosome as being a part of the exosome. It is preferred for the light to be irradiated to the cell intermittently rather than continually in order to deliver the cargo protein inside the exosome more efficiently. That is, when the light is irradiated intermittently, the conjugation and separation of the first photo-specific binding protein and the second photo-specific binding protein repeat so that the probability that the cargo protein is introduced into the exosome can be increased.

In the meantime, the wavelength of the light enough to induce the binding of the first photo-specific binding protein with the second photo-specific binding protein varies from the kinds of the first and the second photo-specific binding proteins. The wavelength of the light that induces the binding of the first photo-specific binding protein and the second photo-specific binding protein depends on the type of the proteins. So, the proper wavelength of the light can be selected as known to those in the art. For example, in order to link CRY2 to CIBN, the light with the wavelength of 460˜490 nm is preferred. If the light is irradiated less than 10 minutes, CRY2 and CIBN are separated from each other. When PhyB is combined with PIF, the light with the wavelength of 650 nm is irradiated for 10 minutes. When the light with the wavelength of 750 nm is irradiated for 5 minutes, PhyB and PIF are separated from each other. When FKF1 is combined with GIGANTEA, the light with the wavelength of 460 nm is irradiated for 30 minutes. In a preferred embodiment of the present invention, in order to induce the binding of CIBN and CRY2, the light with the wavelength of 460˜490 nm was irradiated.

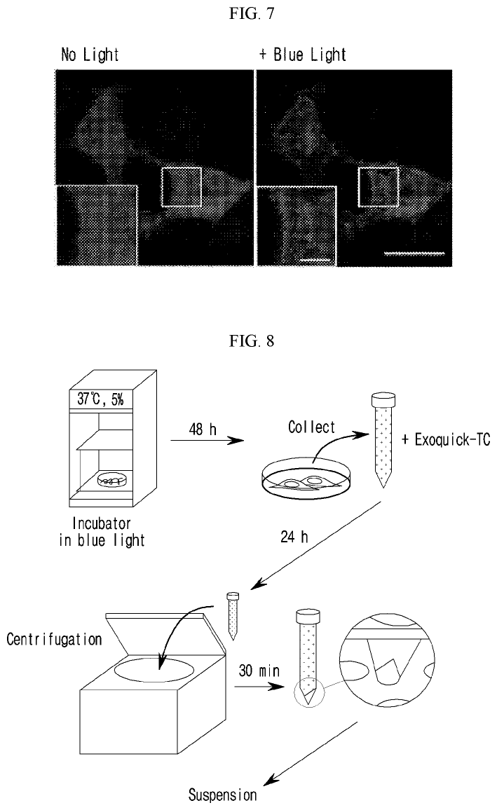

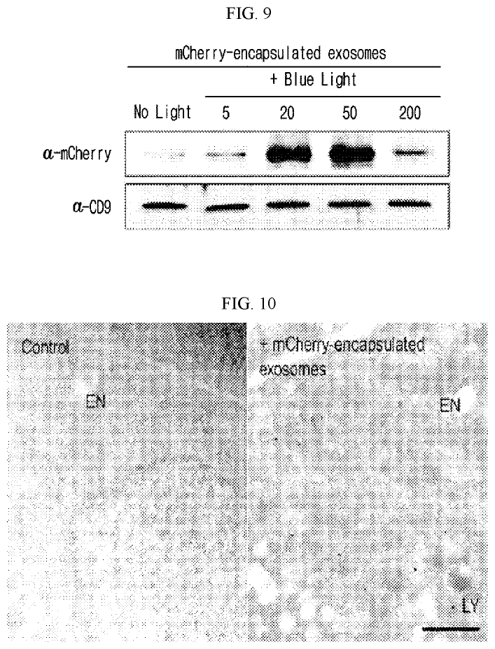

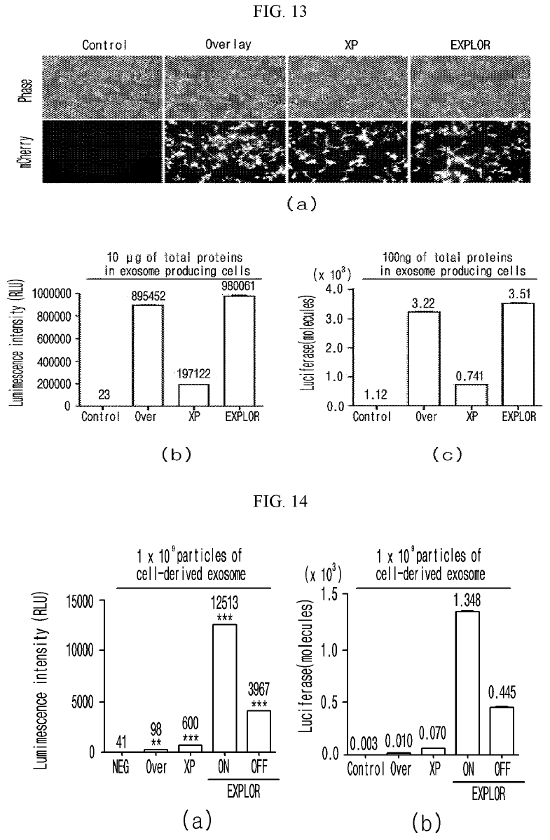

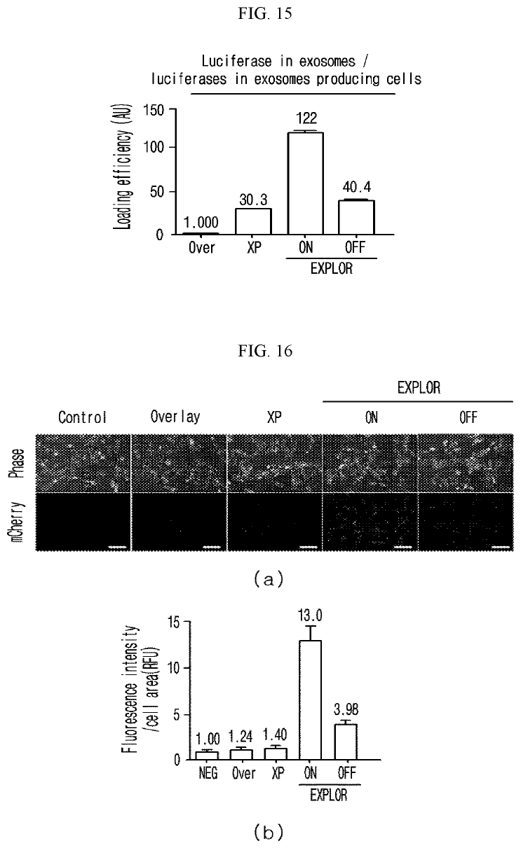

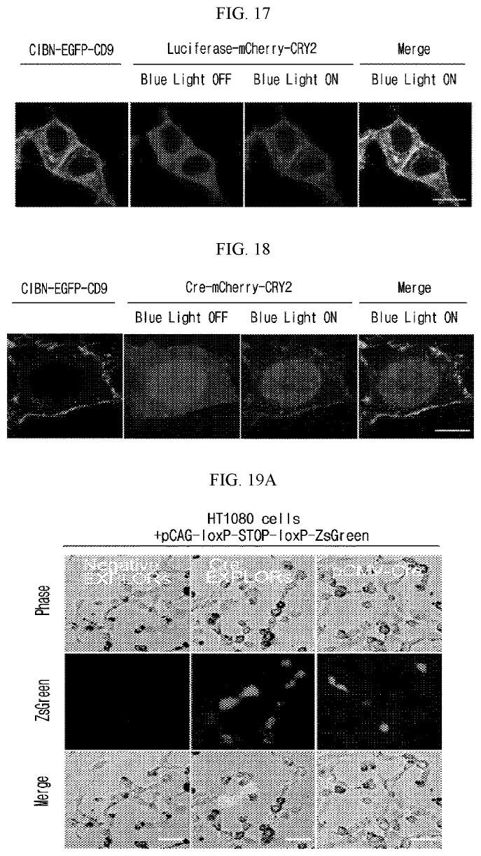

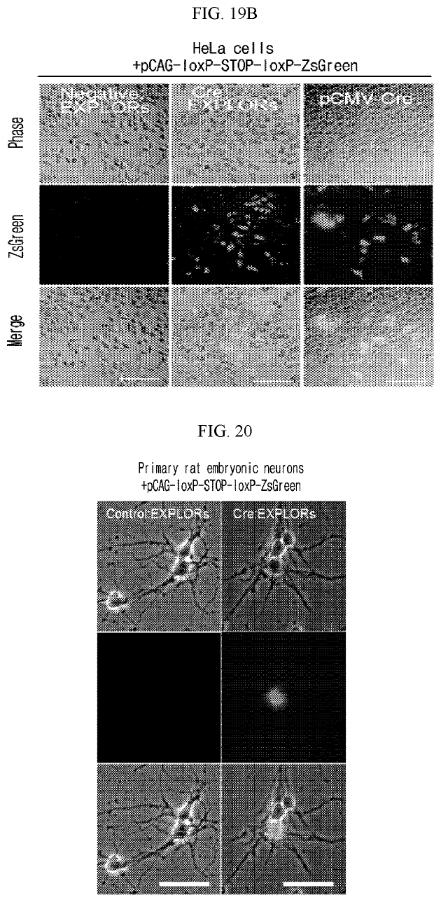

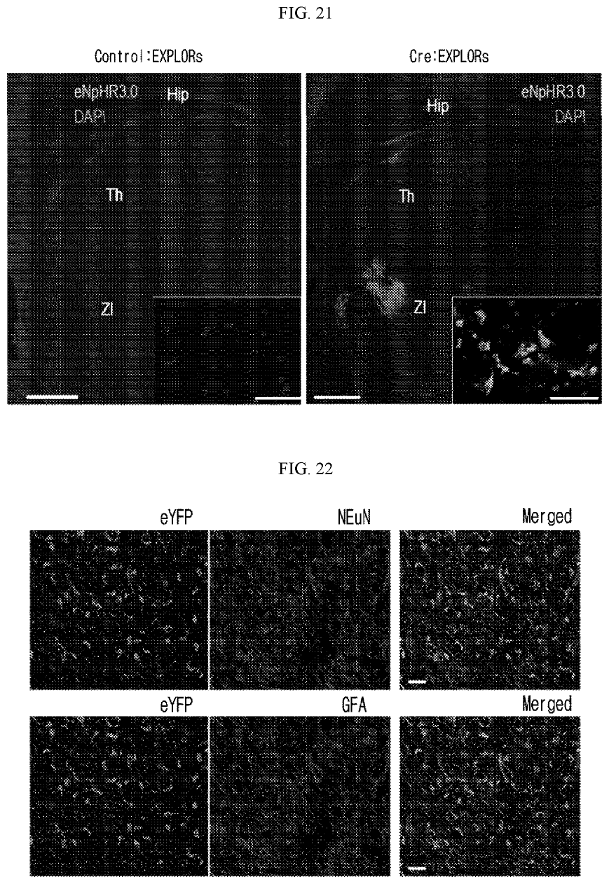

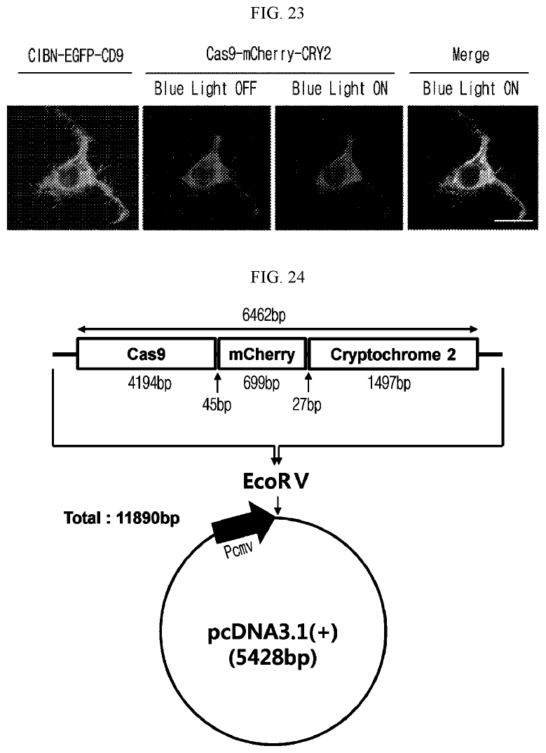

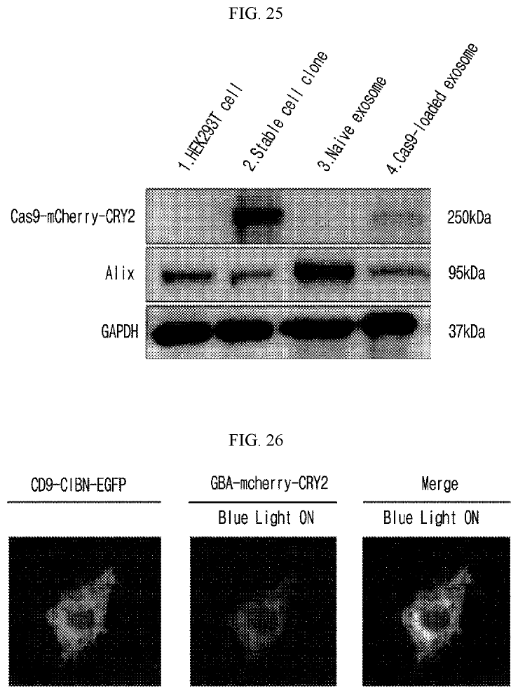

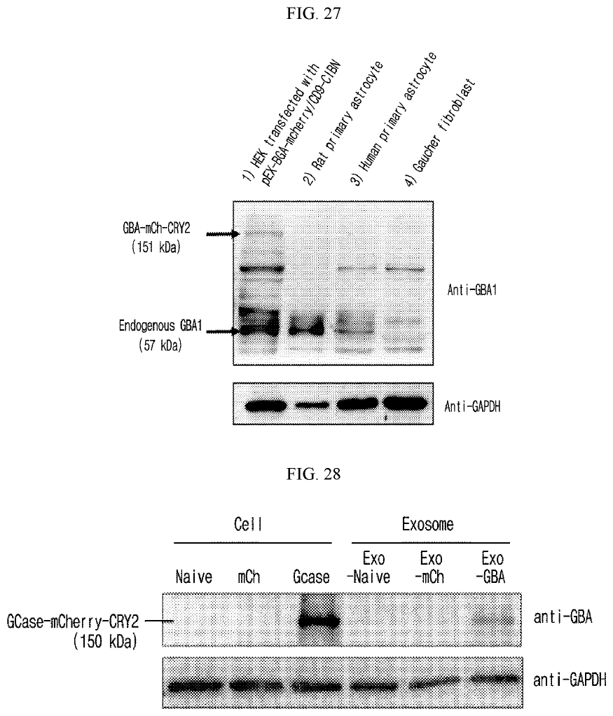

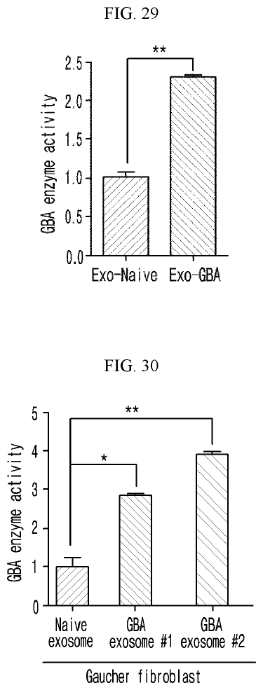

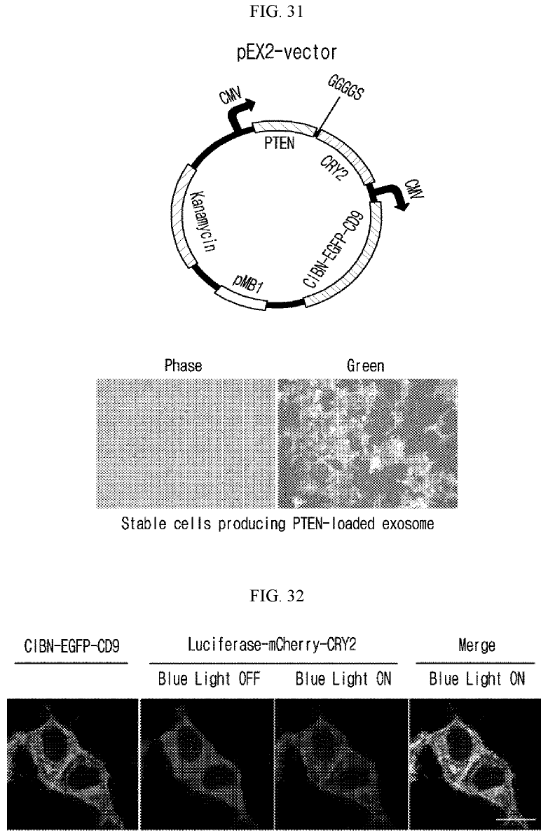

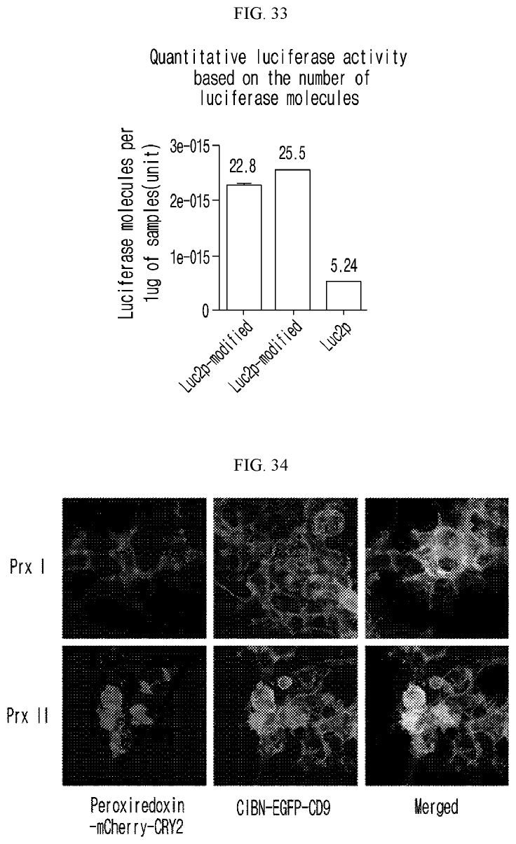

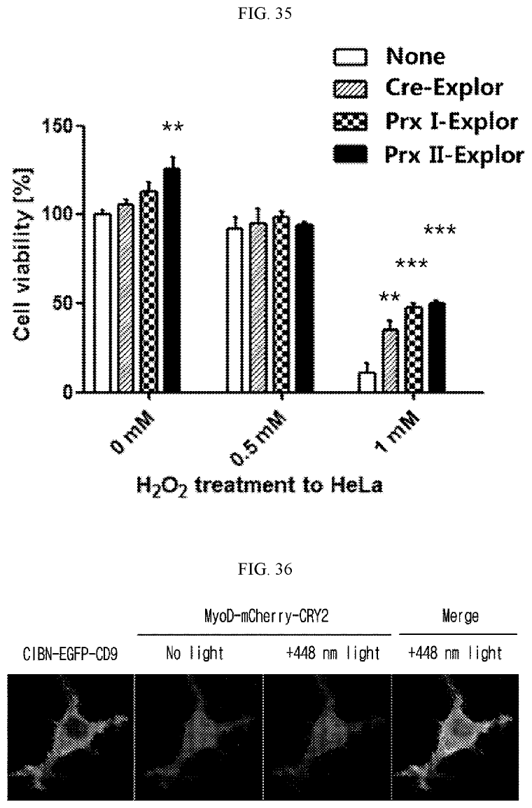

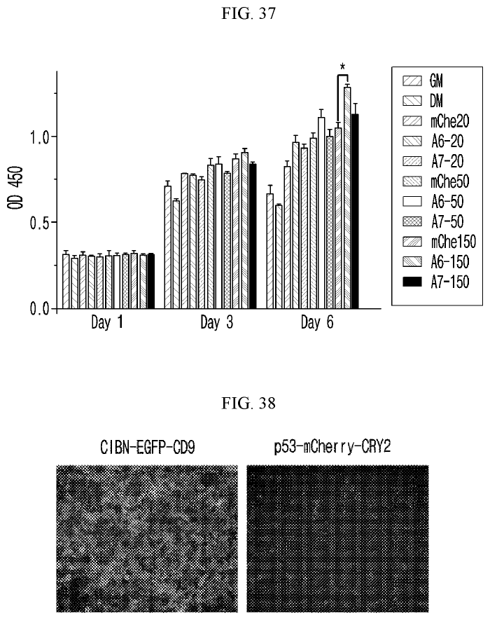

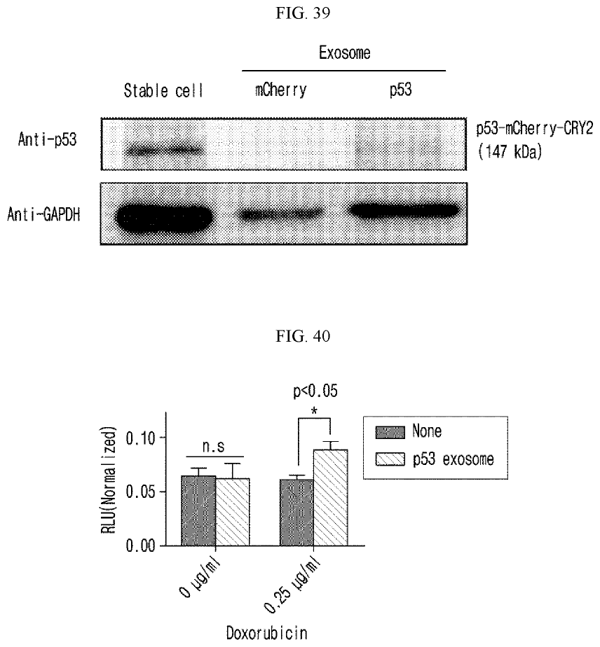

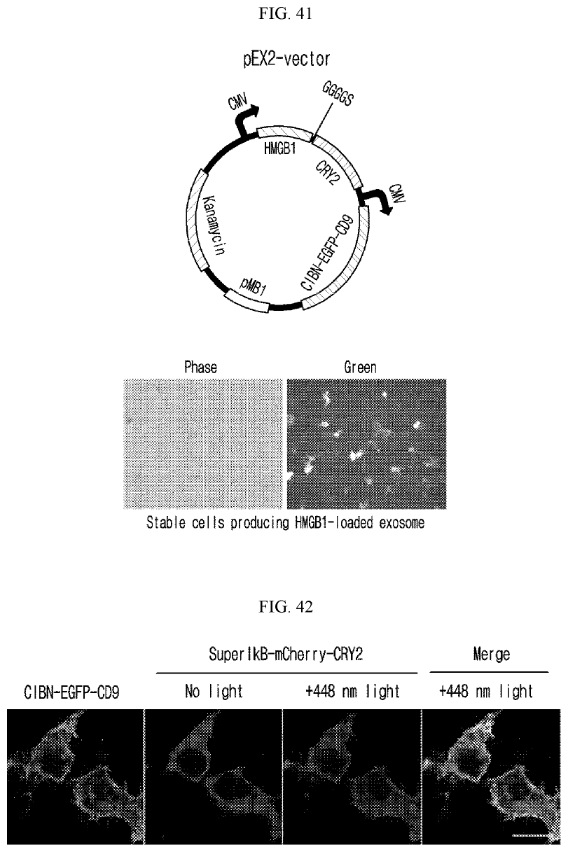

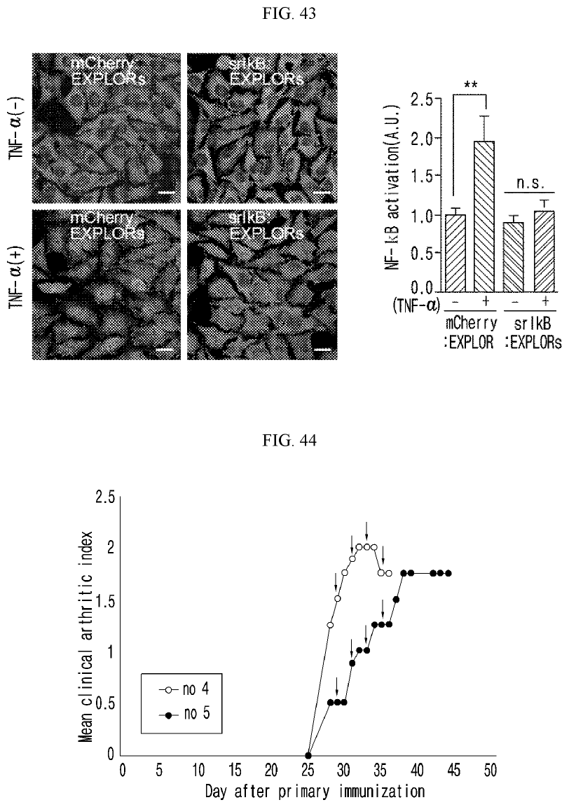

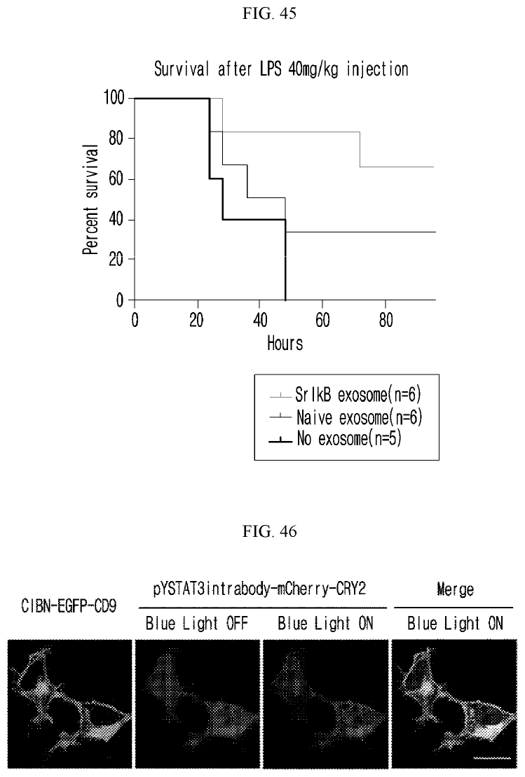

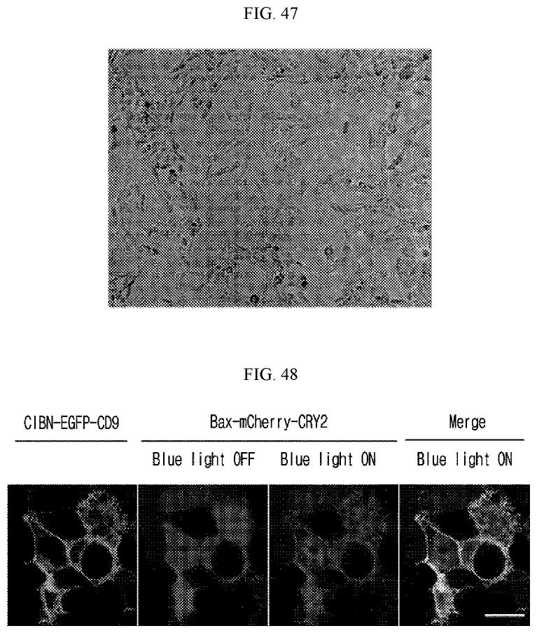

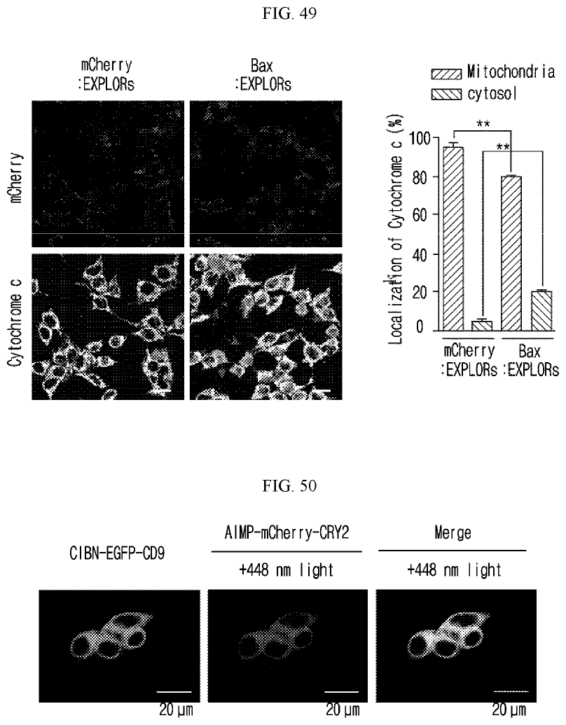

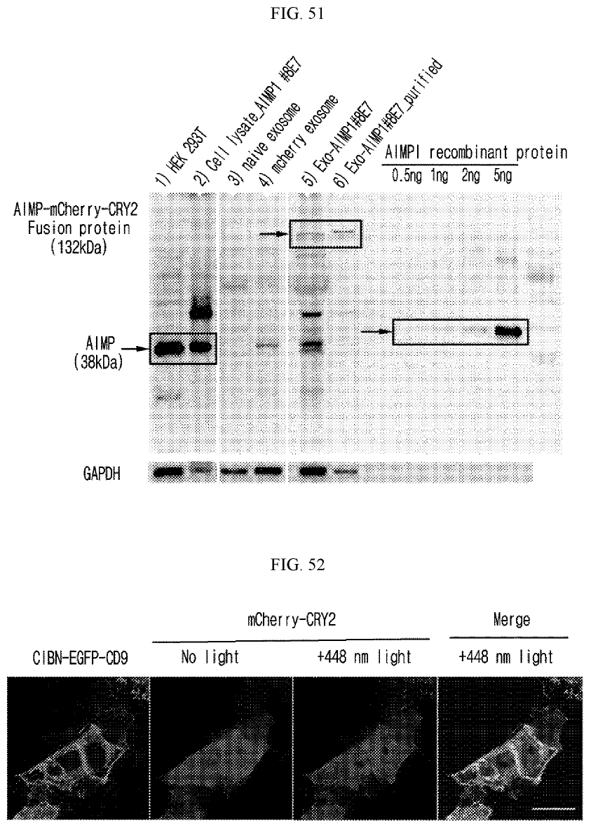

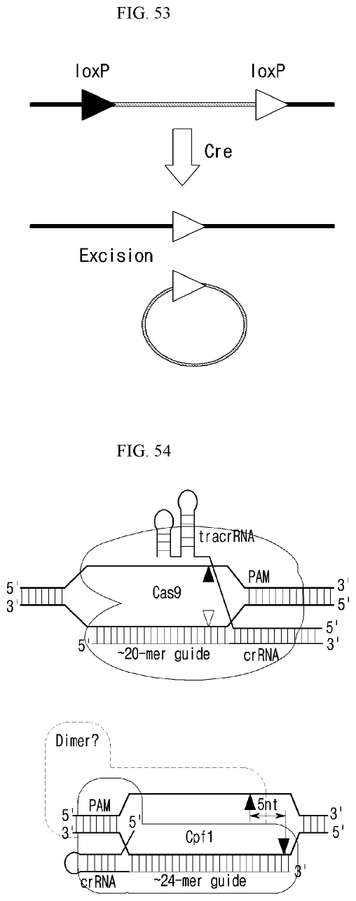

In a preferred embodiment of the present invention, the CRY2/mCherry fusion protein and the CIB/CD9 fusion protein were expressed in HEK293T, the immortalized cell line producing a large amount of exosome. As a result, the distribution of mCherry protein uniformly distributed in cytosol was found to be in cell membrane and endosome-like structure membrane when the blue light was irradiated (FIG. 7). Similar results were observed when the FKF1/mCherry fusion protein and the GIGANTEA/CD9 fusion protein were expressed in HEK293T cells (FIG. 12). The CRY2/mCherry fusion protein and the CIBN/CD9 fusion protein were expressed in HEK293T cells, followed by irradiation with the blue light with regulating the intensity of the light. As a result, when the light was irradiated with the intensity of 20˜50 μW, the level of mCherry protein collected in exosome was the highest (FIG. 9). The exosomes isolated from the cells were treated to HT1080 cells at the concentration of approximately 250 μg/ml. As a result, the exosomes did not show any specific cytotoxicity against the HT1080 cells and it was confirmed that the mCherry protein was delivered in the cytosol thereof (FIG. 10).

To compare the efficiency of introducing the cargo protein in exosome and the efficiency of exosome transfer to the target cell with those of the conventional methods, XPACK vector was used for the conventional method and the expression vectors of the CRY2/mCherry fusion protein and the CIBN/CD9 fusion protein were introduced in HEK293T cells. Then, the production of the cargo protein in exosome was compared. As a result, it was confirmed that the introduction efficiency was remarkably high when the method of the present invention was used (FIG. 15). The exosome separated from the exosome production cell was treated to the target cell (HeLa) to compare the expression of the cargo protein. When the exosome separated by the method of the present invention was used, the expression of the cargo protein was the highest in the target cell (FIG. 16).

In another preferred embodiment of the present invention, the present invention provides a vector for the production of exosome comprising (a) the first expression vector containing the polynucleotide encoding the fusion protein of the exosome specific marker and the first photo-specific binding protein (the first fusion protein); and (b) the second expression vector containing the multicloning site to which the polynucleotide encoding the cargo protein can be introduced and the polynucleotide encoding the second photo-specific binding protein to be linked to the first photo-specific binding protein above.

In the vector for the production of exosome provided by the present invention, the exosome specific marker, the first photo-specific binding protein, the exosome production cell, and the second photo-specific binding protein are same as described above.

The term “transformed cells for exosome production” in this invention indicates the cells capable of producing exosome by expressing the first fusion protein wherein the polynucleotide encoding the fusion protein (the first fusion protein) of the exosome specific marker and the first photo-specific binding protein is introduced.

In this invention, the second expression vector includes a polynucleotide encoding the second photo-specific binding protein and a neighboring multicloning site. When a polynucleotide encoding a cargo protein is inserted in the multicloning site, it is expressed as the fusion protein comprising the second photo-specific binding protein and the cargo protein (the second fusion protein).

The vector for preparing exosome provided by the present invention can contain one or more kinds of constituents, solutions, or devices usable not only for the transformed cells for exosome production and the expression vector; but also for the introduction of the expression vector; for the culture of the transformed cells for exosome production; and for the separation and purification of the exosome produced from the transformed cells for exosome production. For example, a buffer proper for the introduction of the expression vector and a medium and a vessel necessary for the culture of the transformed cells for exosome production can be additionally included.

The term “Cas protein” in this invention indicates the essential protein in CRISPR/Cas system which form active endonuclease or nickase when Cas protein form the complex with two RNA called CRSPR RNA (crRNA) and trans-activating crRNA (tracrRNA).

The term “guide RNA” in this invention indicates target DNA-specific RNA which is able to form complex with Cas protein and guides Cas protein to target DNA.

In this invention, aforementioned guide RNA is able to be made by two RNA, CRISPR RNA (crRNA) and transactivating crRNA (tracrRNA) or single-chain RNA (sgRNA) by fusing the essential parts of crRNA and tracrRNA.

Aforementioned guide RNA is able to be dual RNA including crRNA and tracrRNA. If aforementioned RNA includes the essential parts and target complementary parts of crRNA and tracrRNA, any guide RNA is being able to be applied in this invention. Aforementioned crRNA is able to hybridize target DNA.

Aforementioned guide RNA is able to include one or more additional nucleotide on 5′ terminal of single-chain guide RNA or crRNA in dual RNA.

Desirably, aforementioned guide RNA is able to include two additional guanine nucleotides on 5′ terminal of single-chain guide RNA or crRNA in dual RNA. Guide RNA is able to be delivered to cell or organism as RNA or guide RNA coding DNA. Guide RNA is able to be separated RNA, RNA included in virus vector, or coded in vector. Desirably, aforementioned vector is not limited but is able to be virus vector, plasmid vector, or agrobacterium vector.

Guide RNA coding DNA is able to be vector including guide RNA coding DNA sequence. For example, guide RNA is able to be delivered to cell or organism by transfecting plasmid DNA that includes isolated guide RNA or guide RNA coding sequence and promoter. By other method, guide RNA can be delivered to cell or organism by using virus-mediated gene delivery.

When guide RNA is transfected into cell or organism as isolated RNA, it can be manufactured by in vitro transcription by using any in vitro transcription systems known in industry. Desirably, guide RNA is delivered to cell as isolated RNA rather than plasmid including guide-RNA coding sequence. The term “isolated RNA” can be replaced by “naked RNA” in this invention. It is able to save cost and time in that isolated RNA does not need cloning process. However, the usage of plasmid DNA or virus-mediated gene delivery for guide RNA transfection is not excluded.

The present invention provides the exosome prepared by the method of the invention in which a cargo protein is included.

In another aspect, the present invention provides an exosome produced by the above method, wherein the Cre recombinase is contained in the exosome.

In another aspect, the present invention provides an exosome prepared by the above method, wherein the Cas9 protein is contained therein.

In another aspect, the present invention provides an exosome produced by the above method, wherein GBA (β-glucocerebrosidase) protein is contained therein.

In another aspect, the present invention provides an exosome produced by the above method, wherein the peroxiredoxin (Prx) I or II protein is contained therein.

In another aspect, the present invention provides an exosome produced by the above method and comprising a protein that inhibits NF-kB.

In another aspect, the present invention provides an exosome prepared by the above method, wherein Bax (Bcl-2-associated X protein) protein is contained therein.

The exosome prepared by the method above contains a fusion protein (the first fusion protein) composed of an exosome specific marker and the first photo-specific binding protein on the plasma membrane thereof and another fusion protein (the second fusion protein) composed of the second photo-specific binding protein that can be conjugated to the first photo-specific binding protein and a cargo protein. So, when such exosome is treated to the target tissue cells, the second fusion protein included in the exosome can be delivered to cytosol of the target tissue cells through the fusion of the plasma membrane.

The said exosome containing a cargo protein can be used for the treatment of various diseases in vivo. For example, exosome containing a protein polymer (for example, antibody, etc.) showing the anticancer activity as a cargo protein is prepared, which is then treated to cancer cells. That is, the exosome can be used as a biocompatible anticancer agent better acting than the conventional liposome.

This invention also provides the pharmaceutical components for inflammatory disease prevention and therapy including exosomes with NF-κB inhibiting protein.

Aforementioned inflammatory diseases are not limited but is preferably exemplified by allergy, dermatitis, atopy, conjunctivitis, periodontitis, rhinitis, otitis media, laryngopharyngitis, tonsillitis, pneumonia, gastric ulcer, gastritis, Crohn's disease, colitis, gout, ankylosing spondylitis, rheumatic fever, lupus, fibromyalgia, psoriatic arthritis, osteoarthritis, rheumatoid arthritis, periarthritis of shoulder, tendonitis, tenosynovitis, peritendinitis, myositis, hepatitis, cystitis, nephritis, sjogren's syndrome, multiple sclerosis, acute and chronic inflammatory diseases, sepsis, and ulcerative colitis, etc.

In the experimental examples in this invention, the present inventors confirmed that transfer of NF-κB activated by TNF-α to nucleus is inhibited by pretreating super-repressor-IκB EXPLOR to HeLa cell to verify inflammation inhibitory effect mediated by TNF-α (FIG. 43. Left). In addition, inhibition of DNA binding of NF-κB activated by TNF-α was confirmed (FIG. 43. Right). Also, the present inventors confirmed that symptom of arthritis is decreased in mouse model which is induced arthritis by collagen through injecting retro-orbital three times to verify inflammation inhibitory effects, and thereby super-repressor-IκB:EXPLOR in this invention can be used as the pharmaceutical components for inflammatory disease prevention and therapy.

This invention also provides the pharmaceutical components for cancer prevention and therapy including exosomes with Bax (Bcl-2 associated X protein).

Aforementioned cancer is not limited but is preferably exemplified by breast cancer, colon cancer, lung cancer, small-cell lung cancer, gastric cancer, liver cancer, blood cancer, bone cancer, pancreatic cancer, skin cancer, head or neck cancer, skin or choroidal melanoma, eye cancer, peritoneal cancer, uterine cancer, ovarian cancer, rectal cancer, anal cancer, and cervical cancer, etc.

In the experimental examples in this invention, the present inventors confirmed that cytochrome c release is increased by pretreating Bax::EXPLOR to HeLa cell, and thereby Bax: EXPLOR can be used as the pharmaceutical components for cancer prevention and therapy.

This invention also provides the pharmaceutical components for anti-oxidation including exosomes with peroxiredoxin (Prx) I or II.

Also, this invention provides the pharmaceutical components for prevention and therapy of reactive oxygen species disease exemplified by cancer, arteriosclerosis, respiratory disease, osteoporosis, obesity, and degenerative dementia including exosomes with peroxiredoxin (Prx) I or II.

Also, this invention provides the cosmetic ingredients for anti-oxidation including exosomes with peroxiredoxin (Prx) I or II.

Also, this invention provides the cosmetic ingredients for anti-aging of skin including exosomes with peroxiredoxin (Prx) I or II.

In the experimental examples in this invention, the present inventors confirmed that cytotoxicity by oxidative stress is inhibited statistically significant by pretreating Prx I/II::EXPLOR to HeLa cell to verify the inhibitory effect on cytotoxicity by oxidative stress induced by H₂O₂, and thereby Prx I/II::EXPLOR can be used as the pharmaceutical components for anti-oxidation or prevention and therapy of reactive oxygen species, or the cosmetic ingredients for anti-oxidation or anti-aging of skin.

This invention also provides the components for creating conditional knockout allele of target gene including exosomes with Cre recombinase.

In the experimental examples of this invention, the present inventors confirmed the expression of ZsGreen reporter protein in Cre::EXPLOR treated HT1080 cell and HeLa cell, with identical results of pCMV-Cre vector transfection as positive control, through detecting ZsGreen reporter expression after transfecting pCAG-loxP-STOP-loxP-ZsGreen encoded DNA into HT1080 and HeLa cell to verify the effect of Cre recombinase (FIGS. 19A and 19B). In addition, the present inventors was able to confirm the ZsGreen expression on Cre::EXPOR treated primary mouse embryo neuron performed by experiment identical to aforementioned (FIG. 20). Also, the present inventors confirmed that EYFP is expressed on Cre::EXPLOR treated group after ventrolateral injection of Cre::EXPLOR on pCAG-lowP-STOP-loxP-eNpHR3.0-EYFP transgenic mouse to verify Cre-EXPLOR function in vivo (FIG. 21). Furthermore, it was confirmed that Cre::EXPLOR mainly targets neuron in mouse brain through merged neuronal region in the results of immunohistochemistry to verify Cre::EXPLOR targeting cell (FIG. 22), and thereby Cre::EXPLOR can be used as the components for creating conditional knockout allele of target gene.

This invention also provides the components for engineering DNA sequence including exosomes with Cas9 protein and target DNA specific guide RNA (gRNA).

Aforementioned components are not limited but preferably induces mutation on normal sequence or proofreads mutation. Mutation can be naturally occurred mutation or induced by pathogenic microbes. In other word, mutation is occurred by infection of pathogenic microbes when pathogenic microbes are detected and it becomes clear that biological sample is infected. Pathogenic microbe is not limited but it can be virus or bacteria.

In the experimental examples in this invention, the present inventors confirmed exosome comprising a CRISPR/Cas9 protein can be prepared with a high yield.

This invention also provides the pharmaceutical components for curing Gaucher disease including exosomes with β-glucocerebrosidase (GBA).

In the experimental examples in this invention, the present inventors confirmed that activity of β-glucocerebrosidase (GBA) is recovered by treating GBA::EXPLOR to cells from Gaucher disease patients (FIG. 30), and thereby GBA::EXPLOR can be used as the pharmaceutical components for curing Gaucher disease.

According to the method for preparing exosome containing a cargo protein of the invention, exosome comprising a cargo protein can be prepared with a high yield. Also, a cargo protein presents as being separated from the membrane of exosome, so that it can be widely applied to treat disease.

### Examples

Practical and presently preferred embodiments of the present invention are illustrative as shown in the following Examples. However, it will be appreciated that those skilled in the art, on consideration of this disclosure, may make modifications and improvements within the spirit and scope of the present invention.

### Example 1: Preparation of Exosome

### <1-1> Confirmation of the Binding of CIBN and CRY2 for the Production of Exosome

PcDNA3.1 (+) vector containing CIBN-EGFP-CD9 gene and pcDNA3.1 (+) vector containing mCherry-CRY2 gene were introduced into HEK293T cells, the exosome production cells, under the light-free condition, followed by culture for 24 hours. The medium was replaced with a serum-free medium, followed by additional culture for 48 hours. Upon completion of the culture, the cells were irradiated with the blue light with the wavelength of 460˜490 nm. The location of red fluorescence shown in mCherry before and after the blue light irradiation was confirmed by using a confocal microscope (FIG. 7).

FIG. 7 is a fluorescence image illustrating the changes in the intracellular location of mCherry protein according to the blue light irradiation in the transformed HEK293T cells introduced with CIBN-EGFP-CD9 gene and mCherry-CRY2 gene. As shown in FIG. 7, before the blue light irradiation that could cause the binding of the photo-specific binding proteins CIBN and CRY2, mCherry protein was evenly distributed in the cytosol. However, after the blue light irradiation, mCherry protein was concentrated in the membrane. This clustering of mCherry protein was analyzed to be caused by the binding of CIBN and CRY2, the photo-specific binding proteins.

### <1-2> Confirmation of the Binding of GIGANTEA and FKF1 for the Production of Exosome

PcDNA3.1 (+) vector containing GIGANTEA-EGFP-CD9 gene and pcDNA3.1 (+) vector containing mCherry-FKF1LOV gene were used in this example. The intracellular Exosome was confirmed by the same manner as described in Example<1-1>. (LOV in the FKF1LOV above is an abbreviation of light-oxygen-voltage domain, which indicates the domain that binds to other proteins by light in FKF1 protein, so FKF1 and FKF1LOV are in fact the same herein).

Like Example <1-1>, as shown in FIG. 12, mCherry protein was evenly distributed in the cytosol before the blue light irradiation that could cause the binding of the photo-specific binding proteins GIGANTEA and FKF1. However, after the blue light irradiation, mCherry protein was concentrated in the membrane. This clustering of mCherry protein was analyzed to be caused by the binding of GIGANTEA and FKF1, the photo-specific binding proteins.

### Example 2: Exosome Production and the Effect of Light Intensity on Exosome Production

Each expression vector respectively containing CIBN-EGFP-CD9 gene and mCherry-CRY2 gene was introduced into HEK293T cells under the LED light with the wavelength of 460 nm at the intensity of 0, 5, 20, 50, and 200 μW, followed by culture for 24 hours. Then, the medium was replaced with a serum-free medium, followed by additional culture for 48 hours. Upon completion of the culture, the culture medium was separated, which was centrifuged (3000×g, 15 minutes) to obtain the supernatant excluding cell debris. ExoQuick-TC Exosome Precipitation Solution (System Biosciences, Mountain View, California, USA) was added to the obtained supernatant at the volume of 5 times the supernatant. After the mixing, centrifugation was performed (1500×g, 30 minutes) to obtain the precipitated exosome. The obtained exosome was suspended in PBS, resulting in the exosome suspension. The exosome suspension was filtered with a 0.2 μm filter using a syringe equipped with a 27-G needle. As a result, exosome in the single size was obtained (FIG. 8). Then, exosome lysate was prepared by using lysis buffer, followed by immune-blotting to compare the amount of mCherry protein in the exosome (FIG. 9).

FIG. 9 is an immunoblot analysis image showing the results of measuring the changes of the content of a cargo protein (mCherry protein) captured in exosome according to the intensity of blue light. As shown in FIG. 9, when the cells were irradiated with blue light at the intensity of 20˜50 μW, the amount of mCherry, the cargo protein, in exosome was the highest. From the above results, it was confirmed that the content of the cargo protein captured in exosome could be regulated by controlling the intensity of the light irradiated to the cells in the course of the binding of the photo-specific binding proteins.

### Example 3: Effect of Exosome Treatment

Each expression vector respectively containing CIBN-EGFP-CD9 gene and mCherry-CRY2 gene was introduced into HEK293T cells under the LED light with the wavelength of 460 nm at the intensity of 50 μW, followed by extracting exosome by the same manner as described in Example 2. The extracted exosome was treated to HT1080 cells at the concentration of 250 μg/ml for 24 hours. The HT1080 cells were fixed on 10% gelatin gel by adding with 0.1 M phosphate buffer (pH 7.4) containing 4% PFA and 0.01% GA. The cells attached on the gelatin gel were cooled for a day by using liquid nitrogen. Thin sections cut in 45 nm by using cryoultramicrotome were obtained at −120° C. The thin sections were immuno-stained by using anti-mCherry antibody and Protein A-gold. MCherry protein was observed with Tecnai G2 Spirit Twin TEM (FIG. 10).

FIG. 10 is an electron micrograph illustrating the results of investigation of the introduction of a cargo protein in target cells after treating the target cells (HT1080) with exosome containing the cargo protein (mCherry), wherein the left indicates the target cells not-treated with exosome and the right indicates the target cells treated with exosome. As shown in FIG. 10, it was confirmed that the cargo protein was transferred into the target cells when the target cells were treated with the exosome of the present invention.

### Example 4: Analysis of Exosome with Cargo Protein

Each expression vector respectively containing CIBN-EGFP-CD9 gene and mCherry-CRY2 gene was introduced into HEK293T cells under the LED light with the wavelength of 460 nm at the intensity of 50 μW, followed by extracting exosome by the same manner as described in Example 2. The extracted exosome was treated to HT1080 cells at the concentration of 250 μg/ml for 24 hours. Then, red fluorescence was confirmed in mCherry protein under a fluorescent microscope and the ratio of dead cells was compared between the cells treated with exosome and the cells not-treated with exosome by LDH cell death assay (FIG. 11).

FIG. 11 is a set of a fluorescence image (a) illustrating the results of investigation of the introduction of a cargo protein in target cells after treating the target cells (HT1080) with exosome containing the cargo protein (mCherry); and a graph (b) illustrating the results of comparison of the ratio of apoptotic cells induced by the treatment of exosome. As shown in FIG. 11, it was confirmed that apoptosis did not induced by the treatment of exosome.

### Example 5: Exosome Production and the Comparison of Introduction Efficiency of a Cargo Protein into the Produced Exosome

### <5-1> Confirmation of Exosome Production Efficiency

To compare the exosome production and the introduction efficiency of a cargo protein into the exosome produced thereby according to the present invention with those of the conventional method, the expression of the cargo protein in the exosome production cells was investigated by measuring the luciferase activity therein.

According to the conventional method, XPACK-Luciferase-mCherry was introduced in HEK293T cells by using XPACK (Systems Biosciences), the commercial vector designed for exosome loading technique (XP). On the other hand, according to the method of the invention, Luciferase-mCherry-CRY2 and CIBN-EGFP-CD9 were introduced in HEK293T cells (EXPLOR). Then, the luciferase activity in both cells was measured to compare the efficiency of the two methods. The luciferase activity was measured according to the manufacturer's instructions (Luciferase Assay Reagent, Promega). The standard curve of the results was plotted, and then the number of exosomes in the cells was quantitatively calculated.

As shown in FIG. 14, it was confirmed that the method using the photo-specific binding proteins CIBN and CRY2 of the present invention was significantly higher in the introduction efficiency into exosome than the conventional method (XP) (FIG. 14).

### <5-2> Expression of a Cargo Protein in the Produced Exosome

The cells of Example <5-1> were cultured for 72 hours, followed by extracting exosome (Exoquick-TC, Systems biosciences). The concentration of the cargo protein included in the exosomes separated by the conventional method (XP) or the method of the present invention was compared indirectly by measuring the luciferase activity therein. As shown in FIG. 15, it was confirmed that the method of the present invention could produce exosome containing a remarkably large amount of the cargo protein than the conventional method (FIG. 15).

### <5-3> Comparison of the Introduction Efficiency of the Cargo Protein

The introduction efficiency (E) of the cargo protein was calculated by the mathematical formula below based on the luciferase activity measured in Examples <5-1> and <5-2>.

E=measured value of luciferase activity in produced exosome/measured value of luciferase activity in exosome production cell [Mathematical Formula 1]

As shown in FIG. 15, it was confirmed that the exosome produced using the binding of CRY2 and CIBN of the present invention exhibited 4 to 120 times higher efficiency than those of the other comparative groups (FIG. 15).

### Example 6: Comparison of Exosome Transfer Efficiency to Target Cells

To compare the exosome transfer efficiency, the target cells were treated with exosome containing the cargo protein. Particularly, HeLa cells were treated with 5×10⁹ exosomes for 24 hours, and then the fluorescence intensity expressed in the cells was measured. As shown in FIG. 16, it was confirmed that the fluorescence intensity in the exosome of the present invention (EXPLOR) was remarkably high (FIG. 16).

Therefore, it was confirmed that the method using the exosome of the present invention could deliver the cargo protein to the target cells more efficiently.

### EXPERIMENTAL EXAMPLES

### Experimental Example 1: MMPs (Matrix Metalloproteinases)

The binding of CIBN and CRY2 in cells expressing CIBN-EGFP-CD9 and MMP-mCherry-Cry2 at 488 nm wavelength blue light, and the loading of MMPs within the exosome is evaluated.

For the massive production of MMPs-loaded exosomes, cells stably expressing CIBN-EGFP-CD9 gene and MMP-mCherry-CRY2 gene are established, and exosomes are isolated and purified by Tangential Flow Filtration (TFF) method from culture supernatant.

Functional analysis of MMPs-loaded exosomes is performed in target cells:
Target cells are treated with the MMPs-loaded exosomes to evaluate the functional enzymatic activity.
Animal models are administered with the MMPs-loaded exosomes by i.p. or i.v. to show therapeutic effect.

### Experimental Example 2: TIMPs (Tissue Inhibitor of Metalloproteinase)

The binding of CIBN and CRY2 in cells expressing CIBN-EGFP-CD9 and TIMP-mCherry-Cry2 at 488 nm wavelength blue light, and the loading of TIMPs within the exosome is evaluated.

For the massive production of TIMPs-loaded exosomes, cells stably expressing CIBN-EGFP-CD9 gene and TIMP-mCherry-CRY2 gene are established, and exosomes are isolated and purified by Tangential Flow Filtration (TFF) method from culture supernatant.

Functional analysis of TIMPs-loaded exosomes is performed in target cells:
Target cells are treated with the TIMPs-loaded exosomes to evaluate the functional enzymatic activity.
Animal models are administered with the TIMPs-loaded exosomes by i.p. or i.v. to show therapeutic effect.

### Experimental Example 3: Caspases

The binding of CIBN and CRY2 in cells expressing CIBN-EGFP-CD9 and caspase-mCherry-Cry2 at 488 nm wavelength blue light, and the loading of caspases within the exosome is evaluated.

For the massive production of caspases-loaded exosomes, cells stably expressing CIBN-EGFP-CD9 gene and caspase-mCherry-CRY2 gene are established, and exosomes are isolated and purified by Tangential Flow Filtration (TFF) method from culture supernatant.

Functional analysis of caspases-loaded exosomes is performed in target cells:
Target cells are treated with the caspases-loaded exosomes to evaluate the functional enzymatic activity.
Animal models are administered with the caspases-loaded exosomes by i.p. or i.v. to show therapeutic effect.

### Experimental Example 4: Cathepsins

The binding of CIBN and CRY2 in cells expressing CIBN-EGFP-CD9 and cathepsin-mCherry-Cry2 at 488 nm wavelength blue light, and the loading of cathepsins within the exosome is evaluated.

For the massive production of cathepsins-loaded exosomes, cells stably expressing CIBN-EGFP-CD9 gene and cathepsin-mCherry-CRY2 gene are established, and exosomes are isolated and purified by Tangential Flow Filtration (TFF) method from culture supernatant.

Functional analysis of cathepsins-loaded exosomes is performed in target cells:
Target cells are treated with the cathepsins-loaded exosomes to show the functional enzymatic activity.
Animal models are administered with the cathepsins-loaded exosomes by i.p. or i.v. to show therapeutic effect.

### Experimental Example 5: Cre Recombinase

### <5-1> Production of Cre Recombinase-Loaded Exosome (Cre::EXPLOR)

### A. Confirmation of Cre Recombinase in Exosome

The present inventor confirmed the binding of CIBN and CRY2 in cells expressing CIBN-EGFP-CD9 and Cre-mCherry-Cry2 to verify exosome loading of Cre-recombinase with amino acids recorded as SEQ ID NO: 9. In particular, HEK293T exosome producing cells were additionally cultured 48 hrs in Dulbecco's modified Eagle's medium (DMEM) without fetal bovine serum (FBS), after 24 hrs culture with transfected pcDNA3.1 (+) vector including CIBN-EGFP-CD9 gene and Cre-mCherry-CRY2 gene in non-light condition. After finish culture, position of red fluorescence from mCherry was investigated by confocal microscopy before and after the irradiation of 488 nm wavelength blue light. This experiment was performed more than five times.

According to the results, binding between Cre-mCherry-CRY2 (red) and CIBN-EGFP-CD9 was confirmed (FIG. 18) and thereby exosome loading of Cre recombinase was verified.

### B. Production of Cre Recombinase-Loaded Exosome (Cre::EXPLOR)

The present inventors performed following experiment to yield the Cre recombinase-loaded exosomes.

In particular, CIBN-EGRP-CD9 gene and Cre-mCherry-CRY2 gene included vectors were transfected on HEK293T exosome producing cells and these cells were cultured 24 hrs. After 24 hrs culture, cells were changed their medium as it without fetal bovine serum (FBS) and additionally cultured during 48 hrs on 50 μW power of 488 nm wavelength blue light. After finishing culture, supernatant removed cell debris was yielded by centrifuge (2000×g, 15 min) from isolated culture medium. The supernatant was added ExoQuick-TC Exosome Precipitation Solution (System Biosciences, Mountain View, California, USA) with five times more volume and mixed during 18 hrs on 4° C. Suspended exosomes were obtained by suspending exosome pellet through centrifugation (1500×g, 30 min) of aforementioned supernatant and ExoQuick-TC mixture (FIG. 8).

In addition, HEK293T exosome producing cells which stably express CIBN-EGFP-CD9 gene and Cre-mCherry-CRY2 gene were cultured in medium without fetal bovine serum during 48˜72 hrs on 50 μW power of 488 nm wavelength blue light. After finishing culture, supernatant removed cell debris was yielded by centrifuge (2000×g, 15 min) from isolated culture medium. To remove particles bigger than 200 nm from supernatant, it filtered by 0.2 μl PES membrane (Corning). Tangential Flow Filtration (TFF) method was applied in identical supernatant to remove particles smaller than 20 nm, condense and refine exosomes from filtrate. Vivaflow 50-100 kDa PES membrane (Sartorius) was used in TFF. Exosomes were condensed and refined by rotation of filtrate under 1.5˜2 air pressure of TFF. Then, exosome concentrate was eliminated liquid by centrifugation (10000˜14000 g, 5 min) on Amicon Ultra-0.5 (100 kDa) (Millipore) filter. Finally, exosomes were obtained by reverse-directional centrifugation (10000˜14000 g, 5 min) with preferable buffer on experimental purpose.

### <5-2> Confirmation of Cre Recombinase Function by Cre Recombinase-Loaded Exosome (Cre::EXPLOR)

### A. Functional Confirmation of Cre::EXPLOR on HT1080 and HeLa Cell

Cre recombinase has the function to recombine DNA on loxP regions. The present inventors performed following experiment to investigate the function of Cre::EXPLOR.

In particular, pCAG-loxP-STOP-loxP-ZsGreen encoded DNA was transfected to HT1080 and HeLa cell and washed after 6 hrs. Then 0.25 mg/ml Cre::EXPLOR or Negative::EXPLOR was treated or pCMV-Cre vector was transfected. After 48 hrs culture, expression of ZsGreen with green fluorescence was investigated. Expression of ZsGreen was confirmed on Cre::EXPLOR treated HT1080 and HeLa cell, unlike Negative::EXPLOR treated HT1080 and HeLa cell, and was similar to the results of pCMV-Cre vector transfection in positive control (FIGS. 19A and 19B).

### B. Confirmation of Cre::EXPLOR's Function on Primary Rat Embryonic Neuron

Following experiment was performed to investigate function of Cre::EXPLOR on primary rat embryonic neuron.

In particular, pCAG-loxP-STOP-loxP-ZsGreen encoded DNA was transfected to primary rat embryonic neurons and washed after 6 hrs. Then they were cultured on 0.15 mg/ml Cre::EXPLOR. After 48 hrs culture, expression of ZsGreen with green fluorescence was investigated. This experiment was performed at least three repeats, and thereby expression of ZsGreen was confirmed on Cre::EXPLOR treated primary rat embryonic neuron (FIG. 20).

### C. Confirmation of Cre::EXPLOR Function on In Vivo Transgenic Mouse

The present inventors were performed following experiment to verify Cre::EXPLOR's function on in vivo.

In particular, 50 μl Cre:EXPLORs (10 mg/mL) was injected by ventrolateral injection to pCAG-loxP-STOP-loxP-eNpHR3.0-EYFP transgenic mouse. After injection, fixed brain slices by 4% formaldehyde were imaged by fluorescence microscopy. Green fluorescence indicates expression of eNpHR3.0-EYFP, and blue fluorescence indicates cell nuclei. eNpHR3.0-EYFP expression on neuron in zona incerta (ZI) of Cre::EXPLOR treated mouse was investigated by confocal microscopy, and thereby EYFP expression was confirmed on Cre::EXPLOR treated groups of pCAG-loxP-STOP-loxP-eNpHR3.0-EYFP transgenic mouse (FIG. 21).

### D. Confirmation of Cre::EXPLOR Target Cell on Transgenic Mouse

To confirm specific cell targeting of Cre::EXPLOR on aforementioned in vivo experiment, immunohistochemistry was performed. NeuN antibody specifically stained neuron, and GFAP antibody specifically stained astrocytes, and thereby it was confirmed that Cre::EXPLOR targets specifically neuron in mouse brain through investigating that merged region mainly was neuron (FIG. 22).

### Experimental Example 6: CRISPR-Cas9

### <6-1> Production of Cas9-Loaded Exosome (Cas9::EXPLOR)

### A. Confirmation of Cas9 within Exosome

The present inventors investigated the binding of CIBN and CRY2 expressing the CIBN-EGFP-CD9 and Cas9-mCherry-CRY2 to confirm the loading of Cas9, which is recorded in amino acid SEQ ID NO: 10.

As described in FIG. 23, Cas9-mCherry-CRY2 inserted pcDNA3.1(+) vector has 11,890 base pair in length, and the three protein parts consist of Cas9 with NLS sequence at 5-terminal, mCherry, and Cryptochrome 2 has 45 and 27 base pairs of linker sequences, respectively. Each protein part has 4194, 699, and 1497 base pairs in lengths, respectively.

In particular, HEK293T exosome producing cells were additionally cultured 48 hrs in Dulbecco's modified Eagle's medium (DMEM) without fetal bovine serum (FBS), after 24 hrs culture with transfected pcDNA3.1(+) vector including CIBN-EGFP-CD9 gene and Cas9-mCherry-CRY2 gene in non-light condition. After finish culture, position of red fluorescence from mCherry was investigated by confocal microscopy before and after the irradiation of 488 nm wavelength blue light. This experiment was performed more than five times, and thereby Cas9 protein is loaded within exosome by confirming that CIBN-EGFP-CD9 binds to Cas9-mCherry-CRY2 by the blue light stimulus (FIG. 23).

### B. Production of Cas9-Loaded Exosome (Cas9::EXPLOR)

The present inventors performed following experiment to yield the Cas9-loaded exosomes.

In particular, CIBN-EGRP-CD9 gene and Cas9-mCherry-CRY2 gene included vectors were transfected on HEK293T exosome producing cells and these cells were cultured 24 hrs. After 24 hrs culture, cells were changed their medium as it without fetal bovine serum (FBS) and additionally cultured during 48 hrs on 50 μW power of 488 nm wavelength blue light. After finishing culture, supernatant removed cell debris was yielded by centrifuge (2000×g, 15 min) from isolated culture medium. The supernatant was added ExoQuick-TC Exosome Precipitation Solution (System Biosciences, Mountain View, California, USA) with five times more volume and mixed during 18 hrs on 4° C. Suspended exosomes were obtained by suspending exosome pellet through centrifugation (1500×g, 30 min) of aforementioned supernatant and ExoQuick-TC mixture (FIG. 8).

In addition, HEK293T exosome producing cells which stably express CIBN-EGFP-CD9 gene and Cas9-mCherry-CRY2 gene were cultured in medium without fetal bovine serum during 48˜72 hrs on 50 μW power of 488 nm wavelength blue light. After finishing culture, supernatant removed cell debris was yielded by centrifuge (2000×g, 15 min) from isolated culture medium. To remove particles bigger than 200 nm from supernatant, it filtered by 0.2 μl PES membrane (Corning). Tangential Flow Filtration (TFF) method was applied in identical supernatant to remove particles smaller than 20 nm, condense and refine exosomes from filtrate. Vivaflow 50-100 kDa PES membrane (Sartorius) was used in TFF. Exosomes were condensed and refined by rotation of filtrate under 1.5˜2 air pressure of TFF. Then, exosome concentrate was eliminated liquid by centrifugation (10000˜14000 g, 5 min) on Amicon Ultra-0.5 (100 kDa) (Millipore) filter. Finally, exosomes were obtained by reverse-directional centrifugation (10000˜14000 g, 5 min) with preferable buffer on experimental purpose. The loading of Cas9 within the exosome was evaluated (FIG. 25).

Functional analysis of Cas9-loaded exosomes is performed in target cells:
Target cells are treated with the Cas9-loaded exosomes to show the functional activity.
Animal models are administered with the Cas9-loaded exosomes by i.p. or i.v. to show therapeutic effect.

### Experimental Example 7: Caspase-Activated DNase (CAD)

The binding of CIBN and CRY2 in cells expressing CIBN-EGFP-CD9 and CAD-mCherry-Cry2 at 488 nm wavelength blue light, and the loading of CAD within the exosome is evaluated.

For the massive production of CAD-loaded exosomes, cells stably expressing CIBN-EGFP-CD9 gene and CAD-mCherry-CRY2 gene are established, and exosomes are isolated and purified by Tangential Flow Filtration (TFF) method from culture supernatant.

Functional analysis of CAD-loaded exosomes is performed in target cells:
Target cells are treated with the CAD-loaded exosomes to show the functional activity.
Animal models are administered with the CAD-loaded exosomes by i.p. or i.v. to show therapeutic effect.

### Experimental Example 8: β-glucocerebrosidase

### <8-1> Production of GBA-Loaded Exosome (GBA::EXPLOR)

### A. Confirmation of GBA within Exosome

The present inventors investigated the binding of CIBN and CRY2 expressing the CIBN-EGFP-CD9 and GBA-mCherry-CRY2 to confirm the loading of GBA, which is recorded in amino acid SEQ ID NO: 12.

TABLE 2

| SEQ. | | |
|---|---|---|
| ID | GENE | Nucleotide sequence |
| 12 | β- | MEFSSPSREECPKPLSRVSIMAGSLTGLLLLQAVS |
| | glucocere- | WASGARPCIPKSFGYSSVVCVCNATYCDSFDPPT |
| | brosidase | FPALGTFSRYESTRSGRRMELSMGPIQANHTGTG |
| | | LLLTLQPEQKFQKVKGFGGAMTDAAALNILALS |
| | | PPAQNLLLKSYFSEEGIGYNIIRVPMASCDFSIRT |
| | | YTYADTPDDFQLHNFSLPEEDTKLKIPLIHRALQL |
| | | AQRPVSLLASPWTSPTWLKTNGAVNGKGSLKGQP |
| | | GDIYHQTWARYFVKFLDAYAEHKLQFWAVTAEN |
| | | EPSAGLLSGYPFQCLGFTPEHQRDFIARDLGPTL |
| | | ANSTHHNVRLLMLDDQRLLLPHWAKVVLTDPE |
| | | AAKYVHGIAVHWYLDFLAPAKATLGETHRLFPN |
| | | TMLFASEACVGSKFWEQSVRLGSWDRGMQYSH |
| | | SIITNLLYHVVGWTDWNLALNPEGGPNWVRNFV |
| | | DSPIIVDITKDTFYKQPMFYHLGHFSKFIPEGSQR |
| | | VGLVASQKNDLDAVALMHPDGSAVVVVLNRSSK |
| | | DVPLTIKDPAVGFLETISPGYSIHTYLWRRQ |

In particular, HEK293T exosome producing cells were additionally cultured 48 hrs in Dulbecco's modified Eagle's medium (DMEM) without fetal bovine serum (FBS), after 24 hrs culture with transfected pcDNA3.1(+) vector including CIBN-EGFP-CD9 gene and GBA-mCherry-CRY2 gene in non-light condition. After finish culture, position of red fluorescence from mCherry was investigated by confocal microscopy before and after the irradiation of 488 nm wavelength blue light. This experiment was performed more than five times, and thereby GBA protein is loaded within exosome by confirming that CIBN-EGFP-CD9 binds to GBA-mCherry-CRY2 by the blue light stimulus (FIG. 26).

### B. Production of GBA-Loaded Exosome (GBA::EXPLOR)

The present inventors performed following experiment to yield the GBA-loaded exosomes.

In particular, CIBN-EGRP-CD9 gene and GBA-mCherry-CRY2 gene included vectors were transfected on HEK293T exosome producing cells and these cells were cultured 24 hrs. After 24 hrs culture, cells were changed their medium as it without fetal bovine serum (FBS) and additionally cultured during 48 hrs on 50 μW power of 488 nm wavelength blue light. After finishing culture, supernatant removed cell debris was yielded by centrifuge (2000×g, 15 min) from isolated culture medium. The supernatant was added ExoQuick-TC Exosome Precipitation Solution (System Biosciences, Mountain View, California, USA) with five times more volume and mixed during 18 hrs on 4° C. Suspended exosomes were obtained by suspending exosome pellet through centrifugation (1500×g, 30 min) of aforementioned supernatant and ExoQuick-TC mixture (FIG. 8).

In addition, HEK293T exosome producing cells which stably express CIBN-EGFP-CD9 gene and GBA-mCherry-CRY2 gene were cultured in medium without fetal bovine serum during 48˜72 hrs on 50 μW power of 488 nm wavelength blue light. After finishing culture, supernatant removed cell debris was yielded by centrifuge (2000×g, 15 min) from isolated culture medium. To remove particles bigger than 200 nm from supernatant, it filtered by 0.2 μl PES membrane (Corning). Tangential Flow Filtration (TFF) method was applied in identical supernatant to remove particles smaller than 20 nm, condense and refine exosomes from filtrate. Vivaflow 50-100 kDa PES membrane (Sartorius) was used in TFF. Exosomes were condensed and refined by rotation of filtrate under 1.5˜2 air pressure of TFF. Then, exosome concentrate was eliminated liquid by centrifugation (10000˜14000 g, 5 min) on Amicon Ultra-0.5 (100 kDa) (Millipore) filter. Finally, exosomes were obtained by reverse-directional centrifugation (10000˜14000 g, 5 min) with preferable buffer on experimental purpose.

### <8-2> Measurement of GBA Expression in GBA-Loaded Exosome Producing Cells

The present inventors performed western blot to measure GBA expression in GBA-loaded exosome.

In particular, CIBN-EGRP-CD9 gene and GBA-mCherry-CRY2 gene included vectors were transfected on HEK293T exosome producing cells and these cells were cultured 24 hrs. The HEK293T cells were lysed using MPER (Mammalian Protein Extraction Reagent) and the proteins were analyzed by western blot. Rat primary astrocyte, human primary astrocyte, and Gaucher disease patient-derived fibroblast where β-glucocerebrosidase is deficient due to GBA gene abnormality were lysed to perform western blot and the proteins were analyzed by western blot.

As a result, endogenous GBA was observed in HEK293T cells including CIBN-EGRP-CD9 gene and GBA-mCherry-CRY2 gene, rat primary astrocyte, human primary astrocyte, except Gaucher disease patient-derived fibroblast (FIG. 27).

In addition, GBA-mCherry-CRY2 fusion protein (151 kDa) was observed in HEK293T cells including CIBN-EGRP-CD9 gene and GBA-mCherry-CRY2 gene, and this presents that GBA-mCherry-CRY2 fusion protein is well expressed in GBA-loaded exosome producing cells (FIG. 28).

### <8-3> Confirmation of GBA Activity on Gaucher Disease Patient-Derived Cells by GBA-Loaded Exosome (GBA::EXPLOR)

### A. Enzyme Activity of GBA within Exosome

The present inventor performed experiment for β-glucocerebrosidase enzyme activity to investigate glucocerebroside degrading activity of GBA within exosome.

In particular, CIBN-EGRP-CD9 gene and GBA-mCherry-CRY2 gene included vectors were transfected on HEK293T exosome producing cells and these cells were cultured 24 hrs. After 24 hrs culture, cells were changed their medium as it without fetal bovine serum (FBS) and additionally cultured during 48 hrs on 50 μW power of 488 nm wavelength blue light. After finishing culture, supernatant removed cell debris was yielded by centrifuge (2000×g, 15 min) from isolated culture medium. The supernatant was added ExoQuick-TC Exosome Precipitation Solution (System Biosciences, Mountain View, California, USA) with five times more volume and mixed during 18 hrs on 4° C. Suspended exosomes were obtained by suspending exosome pellet through centrifugation (1500×g, 30 min) of aforementioned supernatant and ExoQuick-TC mixture.

In addition, HEK293T exosome producing cells which stably express CIBN-EGFP-CD9 gene and GBA-mCherry-CRY2 gene were cultured in medium without fetal bovine serum during 48˜72 hrs on 50 μW power of 488 nm wavelength blue light. After finishing culture, supernatant removed cell debris was yielded by centrifuge (2000×g, 15 min) from isolated culture medium. To remove particles bigger than 200 nm from supernatant, it filtered by 0.2 μl PES membrane (Corning). Tangential Flow Filtration (TFF) method was applied in identical supernatant to remove particles smaller than 20 nm, condense and refine exosomes from filtrate. Vivaflow 50-100 kDa PES membrane (Sartorius) was used in TFF. Exosomes were condensed and refined by rotation of filtrate under 1.5˜2 air pressure of TFF. Then, exosome concentrate was eliminated liquid by centrifugation (10000˜14000 g, 5 min) on Amicon Ultra-0.5 (100 kDa) (Millipore) filter. Finally, exosomes were obtained by reverse-directional centrifugation (10000˜14000 g, 5 min). The exosomes were lysed using MPER (Mammalian Protein Extraction Reagent) and the proteins were analyzed.

Increased β-glucocerebrosidase enzyme activity of GBA-loaded GBA::EXPLOR was observed comparing to mCherry-loaded exosomes, and thereby active GBA loading on exosome was confirmed (FIG. 29).

### B. Enzyme Activity of β-Glucocerebrosidase (GBA) on Gaucher Disease Patients-Derived Cells

The present inventors performed following experiment to confirm the recovery of 3-glucocerebrosidase enzyme activity on Gaucher disease patients-derived cells when treated with GBA::EXPLOR.

Gaucher disease patients-derived fibroblast was cultivated at the density of 2×10⁵ cells in 60 mm dish. Then, mCherry::EXPLORs (2×10⁹ exosomes) or GBA::EXPLORs (1.2×10¹⁰ exosomes) were treated to Gaucher disease patients-derived fibroblast cultured in serum-free DMEM medium. Hydrolysis activity of GBA-mCh-CRY2 was measured by detecting the fluorescence using substrate 4-methylumbelliferyl-β-D-glucopyranoside (4-MUG; Sigma). Enzyme reaction was performed on 0.2 ml of 0.2 M citrate phosphate buffer (pH 0.5) containing 50 μl cell lysate of 0.15% (v/v) Triton X-100 (Sigma), 0.8% (w/v) sodium taurocholate (Sigma), 10 mM 4-MUG. After 1 hr incubation at 37° C., the enzyme activity was stopped using 100 μl of 0.1 M glycine, 0.1 M NaOH (pH 10.3). Enzyme reaction product, 4-methylumbelliferone (4-MU) was measured at excitation 365 nm, emission 460 nm condition.

As a result, β-glucocerebrosidase enzyme activity of GBA-loaded GBA::EXPLOR treated Gaucher disease patients-derived cells was recovered (FIG. 30).

### Experimental Example 9: Mitogen Activated Kinases: p38 MAP Kinase

The present inventors confirm the binding of CIBN and CRY2 in cells expressing CIBN-EGFP-CD9 and p38 MAP kinase-mCherry-Cry2 at 488 nm wavelength blue light, and verify the loading of p38 MAP kinase within exosome.

For the massive production of p38 MAP kinase-loaded exosomes, cells stably expressing CIBN-EGFP-CD9 gene and p38 MAP kinase-mCherry-CRY2 gene are established, and exosomes are isolated and purified by Tangential Flow Filtration (TFF) method from culture supernatant.

Functional analysis of p38 MAP kinase-loaded exosomes is performed in target cells

Treatment of p38 MAP kinase-loaded exosomes to target cells shows the functional activity.

Administration of p38 MAP kinase-loaded exosomes by i.p. or i.v. to animal model shows therapeutic effect.

### Experimental Example 10: Inhibitor Kappa B Kinase (IKK)

The binding of CIBN and CRY2 in cells expressing CIBN-EGFP-CD9 and IKK-mCherry-Cry2 at 488 nm wavelength blue light, and the loading of IKK within the exosome is evaluated.

For the massive production of IKK-loaded exosomes, cells stably expressing CIBN-EGFP-CD9 gene and IKK-mCherry-CRY2 gene are established, and exosomes are isolated and purified by Tangential Flow Filtration (TFF) method from culture supernatant.

Functional analysis of IKK kinase-loaded exosomes is performed in target cells:
Target cells are treated with the IKK-loaded exosomes to show the functional activity.
Animal models are administered with the IKK-loaded exosomes by i.p. or i.v. to show therapeutic effect.

### Experimental Example 11: PTEN Phosphatase

The present inventors confirmed the binding of CIBN and CRY2 in cells expressing CIBN-EGFP-CD9 and PTEN-Cry2 at 488 nm wavelength blue light, and the loading of PTEN within exosome.

For the massive production of PTEN-loaded exosomes, cells stably expressing CIBN-EGFP-CD9 gene and PTEN-CRY2 gene were established (FIG. 31), and exosomes were isolated and purified by Tangential Flow Filtration (TFF) method from culture supernatant.

Functional analysis of PTEN kinase-loaded exosomes is performed in target cells

Treatment of PTEN-loaded exosomes to target cells shows the functional activity.

Administration of PTEN-loaded exosomes by i.p. or i.v. to animal model shows therapeutic effect.

### Experimental Example 12: Janus Kinase (JNK)

The binding of CIBN and CRY2 in cells expressing CIBN-EGFP-CD9 and JNK-mCherry-Cry2 at 488 nm wavelength blue light, and the loading of JNK within the exosome is evaluated.

For the massive production of JNK-loaded exosomes, cells stably expressing CIBN-EGFP-CD9 gene and JNK-mCherry-CRY2 gene are established, and exosomes are isolated and purified by Tangential Flow Filtration (TFF) method from culture supernatant.

Functional analysis of JNK-loaded exosomes is performed in target cells:
Target cells are treated with the JNK-loaded exosomes to show the functional activity.
Animal models are administered with the JNK-loaded exosomes by i.p. or i.v. to show therapeutic effect.

### Experimental Example 13: Ubiquitin Ligases

The binding of CIBN and CRY2 in cells expressing CIBN-EGFP-CD9 and Ubiquitin ligase-mCherry-Cry2 at 488 nm wavelength blue light, and the loading of Ubiquitin ligases within the exosome is evaluated.

For the massive production of Ubiquitin ligase-loaded exosomes, cells stably expressing CIBN-EGFP-CD9 gene and Ubiquitin ligase-mCherry-CRY2 gene are established, and exosomes are isolated and purified by Tangential Flow Filtration (TFF) method from culture supernatant.

Functional analysis of Ubiquitin ligase-loaded exosomes is performed in target cells:
Target cells are treated with the Ubiquitin ligase-loaded exosomes to show the functional activity.
Animal models are administered with the Ubiquitin ligase-loaded exosomes by i.p. or i.v. to show therapeutic effect.

### Experimental Example 14: Luciferase

The present inventors confirmed the binding of CIBN and CRY2 in cells expressing CIBN-EGFP-CD9 and luciferase-mcherry-Cry2 at 488 nm wavelength blue light, and the loading of luciferase within exosome (FIG. 32).

For the massive production of luciferase-loaded exosomes, cells stably expressing CIBN-EGFP-CD9 gene and luciferase-mcherry-CRY2 gene were established, and exosomes were isolated and purified by Tangential Flow Filtration (TFF) method from culture supernatant.

Quantitative luciferase activity based on the number of luciferase molecules was analyzed (FIG. 33).

### Experimental Example 15: Peroxiredoxin

### <15-1> Production of Prx I or Prx II-Loaded Exosome (Prx I/II:EXPLOR)

### A. Confirmation of Prx I/II within Exosome

The present inventors investigated the binding of CIBN and CRY2 expressing the CIBN-EGFP-CD9 and Prx I/II-mCherry-CRY2 to confirm the loading of Prx I or Prx II, which is recorded in amino acid SEQ ID NO: 7 or 8.

In particular, HEK293T exosome producing cells were additionally cultured 48 hrs in Dulbecco's modified Eagle's medium (DMEM) without fetal bovine serum (FBS), after 24 hrs culture with transfected pcDNA3.1(+) vector including CIBN-EGFP-CD9 gene and Prx I/II-mCherry-CRY2 gene in non-light condition. After finish culture, position of red fluorescence from mCherry was investigated by confocal microscopy before and after the irradiation of 488 nm wavelength blue light. This experiment was performed more than five times, and thereby aggregation of Prx I/II protein by blue light stimulation was confirmed (FIG. 34). Therefore, Prx I/II protein was loaded in exosome by confirming the co-localization (yellow) of Prx I/II-mCherry-CRY2 (red) and CIBN-EGFP-CD9 (green).

### B. Production of Prx I/II::EXPLOR

The present inventors performed following experiment to yield the Prx I/II-loaded exosomes.

In particular, CIBN-EGRP-CD9 gene and Prx I/II-mCherry-CRY2 gene included vectors were transfected on HEK293T exosome producing cells and these cells were cultured 24 hrs. After 24 hrs culture, cells were changed their medium as it without fetal bovine serum (FBS) and additionally cultured during 48 hrs on 50 μW power of 488 nm wavelength blue light. After finishing culture, supernatant removed cell debris was yielded by centrifuge (2000×g, 15 min) from isolated culture medium. The supernatant was added ExoQuick-TC Exosome Precipitation Solution (System Biosciences, Mountain View, California, USA) with five times more volume and mixed during 18 hrs on 4° C. Suspended exosomes were obtained by suspending exosome pellet through centrifugation (1500×g, 30 min) of aforementioned supernatant and ExoQuick-TC mixture (FIG. 8).

### <15-2> Confirmation of Inhibition Effect on Oxidative Stress-Induced Cytotoxicity by Prx I/II::EXPLOR

The present inventors performed following experiment to confirm the inhibition effect on oxidative stress-induced cytotoxicity by Prx I/II::EXPLOR.

In particular, after changing the serum-free media of HeLa cells, 100 μg/mL of Prx I/IL::EXPLORs was treated and cultivated for 18 hrs. H₂O₂ (0, 0.5, 1 mM) was treated and cultivated for additional 8 hrs. WST assays were used to analyze the cell viability.

Due to pretreat of Prx I/II::EXPLORs, the oxidative stress-induced cytotoxicity was significantly inhibited (FIG. 35).

### Experimental Example 16: NF-κB

The binding of CIBN and CRY2 in cells expressing CIBN-EGFP-CD9 and NF-κB-mCherry-Cry2 at 488 nm wavelength blue light, and the loading of NF-κB within the exosome is evaluated.

For the massive production of NF-κB-loaded exosomes, cells stably expressing CIBN-EGFP-CD9 gene and NF-κB-mCherry-CRY2 gene are established, and exosomes are isolated and purified by Tangential Flow Filtration (TFF) method from culture supernatant.

Functional analysis of NF-κB-loaded exosomes is performed in target cells:
Target cells are treated with the NF-κB-loaded exosomes to show the functional activity.
Animal models are administered with the NF-κB-loaded exosomes by i.p. or i.v. to show therapeutic effect.

### Experimental Example 17: MyoD

The present inventors confirmed the binding of CIBN and CRY2 in cells expressing CIBN-EGFP-CD9 and MyoD-mcherry-Cry2 at 488 nm wavelength blue light (FIG. 36), and verify the loading of MyoD within exosome.

For the massive production of MyoD-loaded exosomes, cells stably expressing CIBN-EGFP-CD9 gene and MyoD-mcherry-CRY2 gene were established, and exosomes were isolated and purified by Tangential Flow Filtration (TFF) method from culture supernatant.

Treatment of MyoD-loaded exosomes to target cells showed the functional activity (FIG. 37).

### Experimental Example 18: Tbx18 (T-Box Transcription Factor 18)

The binding of CIBN and CRY2 in cells expressing CIBN-EGFP-CD9 and Tbx18-mCherry-Cry2 at 488 nm wavelength blue light, and the loading of Tbx18 within the exosome is evaluated.

For the massive production of Tbx18-loaded exosomes, cells stably expressing CIBN-EGFP-CD9 gene and Tbx18-mCherry-CRY2 gene are established, and exosomes are isolated and purified by Tangential Flow Filtration (TFF) method from culture supernatant.

Functional analysis of Tbx18-loaded exosomes is performed in target cells:
Target cells are treated with the Tbx18-loaded exosomes to show the functional activity.
Animal models are administered with the Tbx18-loaded exosomes by i.p. or i.v. to show therapeutic effect.

### Experimental Example 19: p⁵³

The present inventors confirmed the binding of CIBN and CRY2 in cells expressing CIBN-EGFP-CD9 and p53-mcherry-Cry2 at 488 nm wavelength blue light (FIG. 38), and the loading of PTEN within exosome (FIG. 39)

For the massive production of p53-loaded exosomes, cells stably expressing CIBN-EGFP-CD9 gene and p53-mcherry-CRY2 gene were established, and exosomes were isolated and purified by Tangential Flow Filtration (TFF) method from culture supernatant.

Treatment of p53-loaded exosomes to target cells showed the transcriptional activity (FIG. 40).

Administration of p53-loaded exosomes by i.p. or i.v. to animal model shows therapeutic effect.

### Experimental Example 20: HMGB1

The present inventors confirmed the binding of CIBN and CRY2 in cells expressing CIBN-EGFP-CD9 and HMGB1-Cry2 at 488 nm wavelength blue light, and the loading of HMGB1 within exosome (FIG. 41).

For the massive production of HMGB1-loaded exosomes, cells stably expressing CIBN-EGFP-CD9 gene and HMGB1-CRY2 gene were established, and exosomes were isolated and purified by Tangential Flow Filtration (TFF) method from culture supernatant.

Functional analysis of HMGB1-loaded exosomes is performed in target cells
Treatment of HMGB1-loaded exosomes to target cells shows the transcriptional activity.
Administration of HMGB1-loaded exosomes by i.p. or i.v. to animal model shows therapeutic effect.

### Experimental Example 21: Super-Repressor IκB

### <21-1> Production of Super-Repressor-IκB-Loaded Exosome (Super-Repressor-IκB:EXPLOR)

### A. Confirmation of Super-Repressor-IκB in Exosome

The present inventors investigated the binding of CIBN and CRY2 expressing the CIBN-EGFP-CD9 and super-repressor-IκB-mCherry-CRY2 to confirm the loading of super-repressor-IκB, which is recorded in amino acid SEQ ID NO: 5.

In particular, HEK293T exosome producing cells were additionally cultured 48 hrs in Dulbecco's modified Eagle's medium (DMEM) without fetal bovine serum (FBS), after 24 hrs culture with transfected pcDNA3.1(+) vector including CIBN-EGFP-CD9 gene and super-repressor-IκB-mCherry-CRY2 gene in non-light condition. After finish culture, position of red fluorescence from mCherry was investigated by confocal microscopy before and after the irradiation of 488 nm wavelength blue light. This experiment was performed more than five times, and thereby aggregation of super-repressor-IκB protein by blue light stimulation was confirmed (FIG. 42). Therefore, super-repressor-IκB protein was loaded in exosome by confirming the co-localization (yellow) of super-repressor-IκB-mCherry-CRY2 (red) and CIBN-EGFP-CD9 (green).

### B. Production of Super-Repressor-IκB::EXPLOR

The present inventors performed following experiment to yield the super-repressor-IκB-loaded exosomes.

In particular, CIBN-EGRP-CD9 gene and super-repressor-IκB-mCherry-CRY2 gene included vectors were transfected on HEK293T exosome producing cells and these cells were cultured 24 hrs. After 24 hrs culture, cells were changed their medium as it without fetal bovine serum (FBS) and additionally cultured during 48 hrs on 50 μW power of 488 nm wavelength blue light. After finishing culture, supernatant removed cell debris was yielded by centrifuge (2000×g, 15 min) from isolated culture medium. The supernatant was added ExoQuick-TC Exosome Precipitation Solution (System Biosciences, Mountain View, California, USA) with five times more volume and mixed during 18 hrs on 4° C. Suspended exosomes were obtained by suspending exosome pellet through centrifugation (1500×g, 30 min) of aforementioned supernatant and ExoQuick-TC mixture (FIG. 8).

In addition, HEK293T exosome producing cells which stably express CIBN-EGFP-CD9 gene and super-repressor-IκB-mCherry-CRY2 gene were cultured in medium without fetal bovine serum during 48˜72 hrs on 50 μW power of 488 nm wavelength blue light. After finishing culture, supernatant removed cell debris was yielded by centrifuge (2000×g, 15 min) from isolated culture medium. To remove particles bigger than 200 nm from supernatant, it filtered by 0.2 μl PES membrane (Corning). Tangential Flow Filtration (TFF) method was applied in identical supernatant to remove particles smaller than 20 nm, condense and refine exosomes from filtrate. Vivaflow 50-100 kDa PES membrane (Sartorius) was used in TFF. Exosomes were condensed and refined by rotation of filtrate under 1.5˜2 air pressure of TFF. Then, exosome concentrate was eliminated liquid by centrifugation (10000˜14000 g, 5 min) on Amicon Ultra-0.5 (100 kDa) (Millipore) filter. Finally, exosomes were obtained by reverse-directional centrifugation (10000˜14000 g, 5 min) with preferable buffer on experimental purpose.

### <21-2> Confirmation of Super-Repressor-IκB:EXPLOR Inhibition Effect of TNF-α Mediated NF-κB Activity

The present inventors performed following experiments to confirm the TNF-α mediated anti-inflammatory effect using super-repressor-IκB:EXPLOR.

In particular, HeLa was cultured in 100 mg/mL of mCherry:EXPLORs or super-repressor-IκB-mCherry:EXPLORs treated culture medium for 3 hrs. Then, TNF-α (10 ng/mL) was treated and incubated for additional 30 minutes. After fixing with 4% paraformaldehyde, NF-κB p65 was stained with Alexa Fluor 488-conjugated antibody and inspected using confocal microscopy. To measure the binding activity of p65/c-Rel (NF-kB), nuclei lysate was used in TransAM NF-kB and AP-1 assay kit (ActiveMotif, Carlsbad, CA, USA) according to manufacturer's protocol. Data was presented average ±SEM (n=3), and applied using Tukey's post hoc test and decided significant group (**, p<0.01) through ANOVA test.

By pretreat of super-repressor-IκB:EXPLOR on HeLa cells, TNF-α-activated NF-κB transport to nucleus and NF-κB DNA binding were inhibited (FIG. 43).

### <21-3> Confirmation of Anti-Inflammatory Effect of Super-Repressor-IκB:EXPLOR on Collagen-Induced Arthritis Animal Model

The present inventors performed the following experiment to confirm the anti-inflammatory effect of super-repressor-IκB:EXPLOR on Collagen-induced arthritis mouse model.

In particular, mostly used rheumatoid arthritis model, collagen-induced arthritis mouse model was developed by immunization through injecting bovine collagen type II and adjuvant to tail subcutaneous tissue of DBA/1. Super repressor IκB:EXPLOR was retro-orbitally injected 4 times to two collagen-induced arthritis mouse models every 2 days. Progression of rheumatoid arthritis symptom was determined by clinical score as listed in Table 3. Mean Clinical Score is average value of clinical scores from mouse feet according to the aforementioned table.

When super repressor IκB:EXPLOR was retro-orbitally injected to collagen-induced arthritis mouse models, the rheumatoid arthritis mouse showed decreased symptom (FIG. 44).

TABLE 3

| Severity | |
|---|---|
| Score | Phenotypic signs |
| 0 | No evidence of erythema and swelling |
| 1 | Erythema and mild swelling confined |
| | to the tarsals or ankle joint |
| 2 | Erythema and mild swelling extending |
| | from the ankle to the tarsals |
| 3 | Erythema and moderate swelling extending |
| | from the ankle to metatarsal joint |
| 4 | Erythema and severe swelling encompass the |
| | ankle, foot and digits, or ankylosis of the limb |

### <21-4> Effect of srIkB-Loaded Exosomes on LPS-Induced Sepsis Model

In addition, when super repressor IκB:EXPLOR was Intraperitoneally injected to LPS-induced sepsis mouse models, the sepsis mouse showed significantly increased survival (FIG. 45).

### Experimental Example 22: pySTAT3 Intrabody

The present inventors confirmed the binding of CIBN and CRY2 in cells expressing CIBN-EGFP-CD9 and pySTAT3-mcherry-Cry2 at 488 nm wavelength blue light, and the loading of pySTAT3 within exosome (FIG. 46).

For the massive production of pySTAT3-loaded exosomes, cells stably expressing CIBN-EGFP-CD9 gene and pySTAT3-mcherry-CRY2 gene were established, and exosomes were isolated and purified by Tangential Flow Filtration (TFF) method from culture supernatant.

Treatment of pySTAT3-loaded exosomes to target cells shows the functional activity.

Administration of pySTAT3-loaded exosomes by i.p. or i.v. to animal model shows therapeutic effect.

### Experimental Example 23: Bcl-2-Associated X Protein

### <23-1> Production of Bax-Loaded Exosome (Bax::EXPLOR)

### A. Confirmation of Bax in Exosome

The present inventors investigated the binding of CIBN and CRY2 expressing the CIBN-EGFP-CD9 and Bax-mCherry-CRY2 to confirm the loading of Bax, which is recorded in amino acid SEQ ID NO: 6.

In particular, HEK293T exosome producing cells were additionally cultured 48 hrs in Dulbecco's modified Eagle's medium (DMEM) without fetal bovine serum (FBS), after 24 hrs culture with transfected pcDNA3.1(+) vector including CIBN-EGFP-CD9 gene and Bax-mCherry-CRY2 gene in non-light condition. After finish culture, position of red fluorescence from mCherry was investigated by confocal microscopy before and after the irradiation of 488 nm wavelength blue light. This experiment was performed more than five times, and thereby aggregation of Bax protein by blue light stimulation was confirmed (FIG. 48). Therefore, Bax protein was loaded in exosome by confirming the binding of Bax-mCherry-CRY2 (red) and CIBN-EGFP-CD9 (green).

### B. Production of Bax::EXPLOR

The present inventors performed following experiment to yield the Bax-loaded exosomes.

In particular, CIBN-EGRP-CD9 gene and Bax-mCherry-CRY2 gene included vectors were transfected on HEK293T exosome producing cells and these cells were cultured 24 hrs. After 24 hrs culture, cells were changed their medium as it without fetal bovine serum (FBS) and additionally cultured during 48 hrs on 50 μW power of 488 nm wavelength blue light. After finishing culture, supernatant removed cell debris was yielded by centrifuge (2000×g, 15 min) from isolated culture medium. The supernatant was added ExoQuick-TC Exosome Precipitation Solution (System Biosciences, Mountain View, California, USA) with five times more volume and mixed during 18 hrs on 4° C. Suspended exosomes were obtained by suspending exosome pellet through centrifugation (1500×g, 30 min) of aforementioned supernatant and ExoQuick-TC mixture (FIG. 8).

In addition, HEK293T exosome producing cells which stably express CIBN-EGFP-CD9 gene and Bax-mCherry-CRY2 gene were cultured in medium without fetal bovine serum during 48˜72 hrs on 50 μW power of 488 nm wavelength blue light. After finishing culture, supernatant removed cell debris was yielded by centrifuge (2000×g, 15 min) from isolated culture medium. To remove particles bigger than 200 nm from supernatant, it filtered by 0.2 μl PES membrane (Corning). Tangential Flow Filtration (TFF) method was applied in identical supernatant to remove particles smaller than 20 nm, condense and refine exosomes from filtrate. Vivaflow 50-100 kDa PES membrane (Sartorius) was used in TFF. Exosomes were condensed and refined by rotation of filtrate under 1.5˜2 air pressure of TFF. Then, exosome concentrate was eliminated liquid by centrifugation (10000˜14000 g, 5 min) on Amicon Ultra-0.5 (100 kDa) (Millipore) filter. Finally, exosomes were obtained by reverse-directional centrifugation (10000˜14000 g, 5 min) with preferable buffer on experimental purpose.

### <23-2> Confirmation of Apoptosis by Bax:EXPLOR

Bax is apoptotic regulator, thus the BAX overexpression release cytochrome c by binding to mitochondrial membrane, and inducing the apoptosis. The present inventors confirmed the excretion of cytochrome c using Bax:EXPLOR.

In particular, HeLa in 0.1 mg/mL of mCherry:EXPLORs or Bax:EXPLORs containing medium was cultured for 12 hrs. After fixing using 4% paraformaldehyde, to measure the excretion of cytochrome c, the HeLa was stained with Alexa Fluor 647-conjugated antibody and imaged using confocal microscope and the ratio of cytochrome c was analyzed by counting the number of cells (Scale bars, 20 μm). Data was presented average ±SEM (n=3), and applied using Tukey's post hoc test and decided significant group (**, p<0.01) through ANOVA test.

As a result, larger amount of cytochrome c release was observed in Bax:EXPLOR treated HeLa than mCherry:EXPLOR treated HeLa (FIG. 49).

### Experimental Example 24: Bcl-xL

The binding of CIBN and CRY2 in cells expressing CIBN-EGFP-CD9 and Bcl-xL-mCherry-Cry2 at 488 nm wavelength blue light, and the loading of Bcl-xL within the exosome is evaluated.

For the massive production of Bcl-xL-loaded exosomes, cells stably expressing CIBN-EGFP-CD9 gene and Bcl-xL-mCherry-CRY2 gene are established, and exosomes are isolated and purified by Tangential Flow Filtration (TFF) method from culture supernatant.

Functional analysis of Bcl-xL-loaded exosomes is performed in target cells:
Target cells are treated with Bcl-xL-loaded exosomes to show the functional activity.
Animal models are administered with Bcl-xL-loaded exosomes by i.p. or i.v. to show therapeutic effect.

### Experimental Example 25: AIMP

The present inventors confirmed the binding of CIBN and CRY2 in cells expressing CIBN-EGFP-CD9 and AIMP-mcherry-Cry2 at 488 nm wavelength blue light (FIG. 50), and the loading of AIMP within exosome (FIG. 51).

For the massive production of AIMP-loaded exosomes, cells stably expressing CIBN-EGFP-CD9 gene and AIMP-mcherry-CRY2 gene were established, and exosomes were isolated and purified by Tangential Flow Filtration (TFF) method from culture supernatant.

Treatment of AMP-loaded exosomes to target cells shows the functional activity.

Administration of AIMP-loaded exosomes by i.p. or i.v. to animal model shows therapeutic effect.

### Experimental Example 26: mCherry (Fluorescent Protein)

The present inventors confirmed the binding of CIBN and CRY2 in cells expressing CIBN-EGFP-CD9 and mCherry-Cry2 at 488 nm wavelength blue light (FIG. 52), and the loading of AIMP within exosome.

For the massive production of mCherry-loaded exosomes, cells stably expressing CIBN-EGFP-CD9 gene and mCherry-CRY2 gene were established, and exosomes were isolated and purified by Tangential Flow Filtration (TFF) method from culture supernatant.

### Experimental Example 27: Nucleic Acid-Binding Proteins

The binding of CIBN and CRY2 in cells expressing CIBN-EGFP-CD9 and Nucleic acid-binding protein-mCherry-Cry2 at 488 nm wavelength blue light, and the loading of Nucleic acid-binding protein within the exosome is evaluated.

For the massive production of Nucleic acid-binding protein-loaded exosomes, cells stably expressing CIBN-EGFP-CD9 gene and Nucleic acid-binding protein-mCherry-CRY2 gene are established, and exosomes are isolated and purified by Tangential Flow Filtration (TFF) method from culture supernatant.

Functional analysis of Nucleic acid-binding protein-loaded exosomes is performed in target cells:
Target cells are treated with Nucleic acid-binding protein-loaded exosomes to show the functional activity.
Animal models are administered with Nucleic acid-binding protein-loaded exosomes by i.p. or i.v. to show therapeutic effect.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

1-12. (canceled)

13. A composition comprising an exosome and a cargo protein in the exosome.

14. The composition according to claim 1, wherein the cargo protein is a free cargo protein in the exosome.

15. The composition according to claim 1, wherein the cargo protein comprises srlkB.

16. The composition according to claim 1, wherein the cargo protein is a free cargo protein in the exosome, and the cargo protein comprises srlkB.


SEQUENCE LISTING (84 sequences)

SEQ ID NO: 1 - protein, 317 aa, Homo sapiens
MFQAAERPQEWAMEGPRDGLKKERLLDDRHDSGLDSMKDEEYEQMVKELQEIRLEPQEVPRGSEPWKQQLTEDGDSFLHLAIIHEEKALTMEVIRQVKGDLAFLNFQNNLQQTPLHLAVITNQPEIAEALLGAGCDPELRDFRGNTPLHLACEQGCLASVGVLTQSCTTPHLHSILKATNYNGHTCLHLASIHGYLGIVELLVSLGADVNAQEPCNGRTALHLAVDLQNPDLVSLLLKCGADVNRVTYQGYSPYQLTWGRPSTRIQQQLGQLTLENLQMLPESEDEESYDTESEFTEFTEDELPYDDCVFGGQRLTL

SEQ ID NO: 2 - protein, 356 aa, Homo sapiens
MAGVACLGKAADADEWCDSGLGSLGPDAAAPGGPGLGAELGPGLSWAPLVFGYVTEDGDTALHLAVIHQHEPFLDFLLGFSAGTEYMDLQNDLGQTALHLAAILGETSTVEKLYAAGAGLCVAERRGHTALHLACRVGAHACARALLQPRPRRPREAPDTYLAQGPDRTPDTNHTPVALYPDSDLEKEEEESEEDWKLQLEAENYEGHTPLHVAVIHKDVEMVRLLRDAGADLDKPEPTCGRSPLHLAVEAQAADVLELLLRAGANPAARMYGGRTPLGSAMLRPNPILARLLRAHGAPEPEGEDEKSGPCSSSSDSDSGDEGDEYDDIVVHSSRSQTRLPPTPASKPLPDDPRPV

SEQ ID NO: 3 - protein, 500 aa, Homo sapiens
MNQRRSESRPGNHRLQAYAEPGKGDSGGAGPLSGSARRGRGGGGAIRVRRPCWSGGAGRGGGPAWAVRLPTVTAGWTWPALRTLSSLRAGPSEPHSPGRRPPRAGRPLCQADPQPGKAARRSLEPDPAQTGPRPARAAGMSEARKGPDEAEESQYDSGIESLRSLRSLPESTSAPASGPSDGSPQPCTHPPGPVKEPQEKEDADGERADSTYGSSSLTYTLSLLGGPEAEDPAPRLPLPHVGALSPQQLEALTYISEDGDTLVHLAVIHEAPAVLLCCLALLPQEVLDIQNNLYQTALHLAVHLDQPGAVRALVLKGASRALQDRHGDTALHVACQRQHLACARCLLEGRPEPGRGTSHSLDLQLQNWQGLACLHIATLQKNQPLMELLLRNGADIDVQEGTSGKTALHLAVETQERGLVQFLLQAGAQVDARMLNGCTPLHLAAGRGLMGISSTLCKAGADSLLRNVEDETPQDLTEESLVLLPFDDLKISGKLLLCTD

SEQ ID NO: 4 - protein, 454 aa, Homo sapiens
MPRCPAGAMDEGPVDLRTRPKAAGLPGAALPLRKRPLRAPSPEPAAPRGAAGLVVPLDPLRGGCDLPAVPGPPHGLARPEALYYPGALLPLYPTRAMGSPFPLVNLPTPLYPMMCPMEHPLSADIAMATRADEDGDTPLHIAVVQGNLPAVHRLVNLFQQGGRELDIYNNLRQTPLHLAVITTLPSVVRLLVTAGASPMALDRHGQTAAHLACEHRSPTCLRALLDSAAPGTLDLEARNYDGLTALHVAVNTECQETVQLLLERGADIDAVDIKSGRSPLIHAVENNSLSMVQLLLQHGANVNAQMYSGSSALHSASGRGLLPLVRTLVRSGADSSLKNCHNDTPLMVARSRRVIDILRGKATRPASTSQPDPSPDRSANTSPESSSRLSSNGLLSASPSSSPSQSPPRDPPGFPMAPPNFFLPSPSPPAFLPFAGVLRGPGRPVPPSPAPGGS

SEQ ID NO: 5 - protein, 317 aa, Homo sapiens
MFQAAERPQEWAMEGPRDGLKKERLLDDRHDAGLDAMKDEEYEQMVKELQEIRLEPQEVPRGSEPWKQQLTEDGDSFLHLAIIHEEKALTMEVIRQVKGDLAFLNFQNNLQQTPLHLAVITNQPEIAEALLGAGCDPELRDFRGNTPLHLACEQGCLASVGVLTQSCTTPHLHSILKATNYNGHTCLHLASIHGYLGIVELLVSLGADVNAQEPCNGRTALHLAVDLQNPDLVSLLLKCGADVNRVTYQGYSPYQLTWGRPSTRIQQQLGQLTLENLQMLPESEDEESYDTESEFTEFTEDELPYDDCVFGGQRLTL

SEQ ID NO: 6 - protein, 192 aa, Homo sapiens
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMG

SEQ ID NO: 7 - protein, 199 aa, Homo sapiens
MSSGNAKIGHPAPNFKATAVMPDGQFKDISLSDYKGKYVVFFFYPLDFTFVCPTEIIAFSDRAEEFKKLNCQVIGASVDSHFCHLAWVNTPKKQGGLGPMNIPLVSDPKRTIAQDYGVLKADEGISFRGLFIIDDKGILRQITVNDLPVGRSVDETLRLVQAFQFTDKHGEVCPAGWKPGSDTIKPDVQKSKEYFSKQK

SEQ ID NO: 8 - protein, 198 aa, Homo sapiens
MASGNARIGKPAPDFKATAVVDGAFKEVKLSDYKGKYVVLFFYPLDFTFVCPTEIIAFSNRAEDFRKLGCEVLGVSVDSQFTHLAWINTPRKEGGLGPLNIPLLADVTRRLSEDYGVLKTDEGIAYRGLFIIDGKGVLRQITVNDLPVGRSVDEALRLVQAFQYTDEHGEVCPAGWKPGSDTIKPNVDDSKEYFSKHN

SEQ ID NO: 9 - protein, 343 aa, unidentified
  source 1..343: note = Bacteriophage P1
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 10 - protein, 469 aa, Streptococcus pyogenes
MHSFPPLLLLLFWGVVSHSFPATLETQEQDVDLVQKYLEKYYNLKNDGRQVEKRRNSGPVVEKLKQMQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 11 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 12 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 13 - protein, 469 aa, Homo sapiens; identical to SEQ ID NO: 10

SEQ ID NO: 14 - protein, 207 aa, Homo sapiens
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 15 - protein, 220 aa, Homo sapiens
MGAAARTLRLALGLLLLATLLRPADACSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDP

SEQ ID NO: 16 - protein, 211 aa, Homo sapiens
MTPWLGLIVLLGSWSLGDWGAEACTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCNCKIKSCYYLPCFVTSKNECLWTDMLSNFGYPGYQSKHYACIRQKGGYCSWYRGWAPPDKSIINATDP

SEQ ID NO: 17 - protein, 224 aa, Homo sapiens
MPGSPRPAPSWVLLLRLLALLRPPGLGEACSCAPAHPQQHICHSALVIRAKISSEKVVPASADPADTEKMLRYEIKQIKMFKGFEKVKDVQYIYTPFDSSLCGVKLEANSQKQYLLTGQVLSDGKVFIHLCNYIEPWEDLSLVQRESLNHHYHLNCGCQITTCYTVPCTISAPNECLWTDWLLERKLYGYQAQHYVCMKHVDGTCSWYRGHLPLRKEFVDIVQP

SEQ ID NO: 18 - protein, 404 aa, Homo sapiens
MADKVLKEKRKLFIRSMGEGTINGLLDELLQTRVLNKEEMEKVKRENATVMDKTRALIDSVIPKGAQACQICITYICEEDSYLAGTLGLSADQTSGNYLNMQDSQGVLSSFPAPQAVQDNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKDSVGVSGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGH

SEQ ID NO: 19 - protein, 452 aa, Homo sapiens
MAAPSAGSWSTFQHKELMAADRGRRILGVCGMHPHHQETLKKNRVVLAKQLLLSELLEHLLEKDIITLEMRELIQAKVGSFSQNVELLNLLPKRGPQAFDAFCEALRETKQGHLEDMLLTTLSGLQHVLPPLSCDYDLSLPFPVCESCPLYKKLRLSTDTVEHSLDNKDGPVCLQVKPCTPEFYQTHFQLAYRLQSRPRGLALVLSNVHFTGEKELEFRSGGDVDHSTLVTLFKLLGYDVHVLCDQTAQEMQEKLQNFAQLPAHRVTDSCIVALLSHGVEGAIYGVDGKLLQLQEVFQLFDNANCPSLQNKPKMFFIQACRGDETDRGVDQQDGKNHAGSPGCEESDAGKEKLPKMRLPTRSDMICGYACLKGTAAMRNTKRGSWYIEALAQVFSERACDMHVADMLVKVNALIKDREGYAPGTEFHRCKEMSEYCSTLCRHLYLFPGHPPT

SEQ ID NO: 20 - protein, 277 aa, Homo sapiens
MENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETDSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYH

SEQ ID NO: 21 - protein, 377 aa, Homo sapiens
MAEGNHRKKPLKVLESLGKDFLTGVLDNLVEQNVLNWKEEEKKKYYDAKTEDKVRVMADSMQEKQRMAGQMLLQTFFNIDQISPNKKAHPNMEAGPPESGESTDALKLCPHEEFLRLCKERAEEIYPIKERNNRTRLALIICNTEFDHLPPRNGADFDITGMKELLEGLDYSVDVEENLTARDMESALRAFATRPEHKSSDSTFLVLMSHGILEGICGTVHDEKKPDVLLYDTIFQIFNNRNCLSLKDKPKVIIVQACRGANRGELWVRDSPASLEVASSQSSENLEEDAVYKTHVEKDFIAFCSSTPHNVSWRDSTMGSIFITQLITCFQKYSWCCHLEEVFRKVQQSFETPRAKAQMPTIERLSMTRYFYLFPGN

SEQ ID NO: 22 - protein, 434 aa, Homo sapiens
MAEDSGKKKRRKNFEAMFKGILQSGLDNFVINHMLKNNVAGQTSIQTLVPNTDQKSTSVKKDNHKKKTVKMLEYLGKDVLHGVFNYLAKHDVLTLKEEEKKKYYDTKIEDKALILVDSLRKNRVAHQMFTQTLLNMDQKITSVKPLLQIEAGPPESAESTNILKLCPREEFLRLCKKNHDEIYPIKKREDRRRLALIICNTKFDHLPARNGAHYDIVGMKRLLQGLGYTVVDEKNLTARDMESVLRAFAARPEHKSSDSTFLVLMSHGILEGICGTAHKKKKPDVLLYDTIFQIFNNRNCLSLKDKPKVIIVQACRGEKHGELWVRDSPASLALISSQSSENLEADSVCKIHEEKDFIAFCSSTPHNVSWRDRTRGSIFITELITCFQKYSCCCHLMEIFRKVQKSFEVPQAKAQMPTIERATLTRDFYLFPGN

SEQ ID NO: 23 - protein, 293 aa, Homo sapiens
MSSASGLRRGHPAGGEENMTETDAFYKREMFDPAEKYKMDHRRRGIALIFNHERFFWHLTLPERRGTCADRDNLTRRFSDLGFEVKCFNDLKAEELLLKIHEVSTVSHADADCFVCVFLSHGEGNHIYAYDAKIEIQTLTGLFKGDKCHSLVGKPKIFIIQACRGNQHDVPVIPLDVVDNQTEKLDTNITEVDAASVYTLPAGADFLMCYSVAEGYYSHRETVNGSWYIQDLCEMLGKYGSSLEFTELLTLVNRKVSQRRVDFCKDPSAIGKKQVPCFASMLTKKLHFFPKSN

SEQ ID NO: 24 - protein, 303 aa, Homo sapiens
MADDQGCIEEQGVEDSANEDSVDAKPDRSSFVPSLFSKKKKNVTMRSIKTTRDRVPTYQYNMNFEKLGKCIIINNKNFDKVTGMGVRNGTDKDAEALFKCFRSLGFDVIVYNDCSCAKMQDLLKKASEEDHTNAACFACILLSHGEENVIYGKDGVTPIKDLTAHFRGDRCKTLLEKPKLFFIQACRGTELDDGIQADSGPINDTDANPRYKIPVEADFLFAYSTVPGYYSWRSPGRGSWFVQALCSILEEHGKDLEIMQILTRVNDRVARHFESQSDDPHFHEKKQIPCVVSMLTKELYFSQ

SEQ ID NO: 25 - protein, 479 aa, Homo sapiens
MDFSRNLYDIGEQLDSEDLASLKFLSLDYIPQRKQEPIKDALMLFQRLQEKRMLEESNLSFLKELLFRINRLDLLITYLNTRKEEMERELQTPGRAQISAYRVMLYQISEEVSRSELRSFKFLLQEEISKCKLDDDMNLLDIFIEMEKRVILGEGKLDILKRVCAQINKSLLKIINDYEEFSKERSSSLEGSPDEFSNGEELCGVMTISDSPREQDSESQTLDKVYQMKSKPRGYCLIINNHNFAKAREKVPKLHSIRDRNGTHLDAGALTTTFEELHFEIKPHDDCTVEQIYEILKIYQLMDHSNMDCFICCILSHGDKGIIYGTDGQEAPIYELTSQFTGLKCPSLAGKPKVFFIQACQGDNYQKGIPVETDSEEQPYLEMDLSSPQTRYIPDEADFLLGMATVNNCVSYRNPAEGTWYIQSLCQSLRERCPRGDDILTILTEVNYEVSNKDDKKNMGKQMPQPTFTLRKKLVFPSD

SEQ ID NO: 26 - protein, 416 aa, Homo sapiens
MDEADRRLLRRCRLRLVEELQVDQLWDALLSRELFRPHMIEDIQRAGSGSRRDQARQLIIDLETRGSQALPLFISCLEDTGQDMLASFLRTNRQAAKLSKPTLENLTPVVLRPEIRKPEVLRPETPRPVDIGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 27 - protein, 521 aa, Homo sapiens
MKSQGQHWYSSSDKNCKVSFREKLLIIDSNLGVQDVENLKFLCIGLVPNKKLEKSSSASDVFEHLLAEDLLSEEDPFFLAELLYIIRQKKLLQHLNCTKEEVERLLPTRQRVSLFRNLLYELSEGIDSENLKDMIFLLKDSLPKTEMTSLSFLAFLEKQGKIDEDNLTCLEDLCKTVVPKLLRNIEKYKREKAIQIVTPPVDKEAESYQGEEELVSQTDVKTFLEALPQESWQNKHAGSNGNRATNGAPSLVSRGMQGASANTLNSETSTKRAAVYRMNRNHRGLCVIVNNHSFTSLKDRQGTHKDAEILSHVFQWLGFTVHIHNNVTKVEMEMVLQKQKCNPAHADGDCFVFCILTHGRFGAVYSSDEALIPIREIMSHFTALQCPRLAEKPKLFFIQACQGEEIQPSVSIEADALNPEQAPTSLQDSIPAEADFLLGLATVPGYVSFRHVEEGSWYIQSLCNHLKKLVPRMLKFLEKTMEIRGRKRTVWGAKQISATSLPTAISAQTPRPPMRRWSSVS

SEQ ID NO: 28 - protein, 373 aa, Homo sapiens
MAENKHPDKPLKVLEQLGKEVLTEYLEKLVQSNVLKLKEEDKQKFNNAERSDKRWVFVDAMKKKHSKVGEMLLQTFFSVDPGSHHGEANLEMEEPEESLNTLKLCSPEEFTRLCREKTQEIYPIKEANGRTRKALIICNTEFKHLSLRYGANFDIIGMKGLLEDLGYDVVVKEELTAEGMESEMKDFAALSEHQTSDSTFLVLMSHGTLHGICGTMHSEKTPDVLQYDTIYQIFNNCHCPGLRDKPKVIIVQACRGGNSGEMWIRESSKPQLCRGVDLPRNMEADAVKLSHVEKDFIAFYSTTPHHLSYRDKTGGSYFITRLISCFRKHACSCHLFDIFLKVQQSFEKASIHSQMPTIDRATLTRYFYLFPGN

SEQ ID NO: 29 - protein, 341 aa, Homo sapiens
MADEKPSNGVLVHMVKLLIKTFLDGIFDDLMENNVLNTDEIHLIGKCLKFVVSNAENLVDDITETAQTAGKIFREHLWNSKKQLSSDISSDGEREANMPGLNIRNKEFNYLHNRNGSELDLLGMRDLLENLGYSVVIKENLTAQEMETALRQFAAHPEHQSSDSTFLVFMSHSILNGICGTKHWDQEPDVLHDDTIFEIFNNRNCQSLKDKPKVIIMQACRGNGAGIVWFTTDSGKAGADTHGRLLQGNICNDAVTKAHVEKDFIAFKSSTPHNVSWRHETNGSVFISQIIYYFREYSWSHHLEEIFQKVQHSFETPNILTQLPTIERLSMTRYFYLFPGN

SEQ ID NO: 30 - protein, 377 aa, Homo sapiens
MAEDKHNKNPLKMLESLGKELISGLLDDFVEKNVLKLEEEEKKKIYDAKLQDKARVLVDSIRQKNQEAGQVFVQTFLNIDKNSTSIKAPEETVAGPDESVGSAATLKLCPHEEFLKLCKERAGEIYPIKERKDRTRLALIICNTEFDHMPPRNGAALDILGMKQLLEGLGYTVEVEEKLTARDMESVLWKFAAREEHKSSDSTFLVFMSHGILDGICGTMHSEEEPDVLPYDTIFRTFNNRNCLSLKDKPKVIIVQACRGANRGELWVSDSPPALADSFSQSSENLEEDAVYKTHVEKDFIAFCSSTPHNVSWRDIKKGSLFITRLITCFQKYAWCCHLEEVFRKVQQSFEKPNVKAQMPTVERLSMTRYFYLFPGN

SEQ ID NO: 31 - protein, 242 aa, Homo sapiens
MSNPRSLEEEKYDMSGARLALILCVTKAREGSEEDLDALEHMFRQLRFESTMKRDPTAEQFQEELEKFQQAIDSREDPVSCAFVVLMAHGREGFLKGEDGEMVKLENLFEALNNKNCQALRAKPKVYIIQACRGEQRDPGETVGGDEIVMVIKDSPQTIPTYTDALHVYSTVEGYIAYRHDQKGSCFIQTLVDVFTKRKGHILELLTEVTRRMAEAELVQEGKARKTNPEIQSTLRKRLYLQ

SEQ ID NO: 32 - protein, 480 aa, Homo sapiens
MIRAAPPPLFLLLLLLLLLVSWASRGEAAPDQDEIQRLPGLAKQPSFRQYSGYLKGSGSKHLHYWFVESQKDPENSPVVLWLNGGPGCSSLDGLLTEHGPFLVQPDGVTLEYNPYSWNLIANVLYLESPAGVGFSYSDDKFYATNDTEVAQSNFEALQDFFRLFPEYKNNKLFLTGESYAGIYIPTLAVLVMQDPSMNLQGLAVGNGLSSYEQNDNSLVYFAYYHGLLGNRLWSSLQTHCCSQNKCNFYDNKDLECVTNLQEVARIVGNSGLNIYNLYAPCAGGVPSHFRYEKDTVVVQDLGNIFTRLPLKRMWHQALLRSGDKVRMDPPCTNTTAASTYLNNPYVRKALNIPEQLPQWDMCNFLVNLQYRRLYRSMNSQYLKLLSSQKYQILLYNGDVDMACNFMGDEWFVDSLNQKMEVQRRPWLVKYGDSGEQIAGFVKEFSHIAFLTIKGAGHMVPTDKPLAAFTMFSRFLNKQPY

SEQ ID NO: 33 - protein, 339 aa, Homo sapiens
MWQLWASLCCLLVLANARSRPSFHPLSDELVNYVNKRNTTWQAGHNFYNVDMSYLKRLCGTFLGGPKPPQRVMFTEDLKLPASFDAREQWPQCPTIKEIRDQGSCGSCWAFGAVEAISDRICIHTNAHVSVEVSAEDLLTCCGSMCGDGCNGGYPAEAWNFWTRKGLVSGGLYESHVGCRPYSIPPCEHHVNGSRPPCTGEGDTPKCSKICEPGYSPTYKQDKHYGYNSYSVSNSEKDIMAEIYKNGPVEGAFSVYSDFLLYKSGVYQHVTGEMMGGHAIRILGWGVENGTPYWLVANSWNTDWGDNGFFKILRGQDHCGIESEVVAGIPRTDQYWEKI

SEQ ID NO: 34 - protein, 463 aa, Homo sapiens
MGAGPSLLLAALLLLLSGDGAVRCDTPANCTYLDLLGTWVFQVGSSGSQRDVNCSVMGPQEKKVVVYLQKLDTAYDDLGNSGHFTIIYNQGFEIVLNDYKWFAFFKYKEEGSKVTTYCNETMTGWVHDVLGRNWACFTGKKVGTASENVYVNIAHLKNSQEKYSNRLYKYDHNFVKAINAIQKSWTATTYMEYETLTLGDMIRRSGGHSRKIPRPKPAPLTAEIQQKILHLPTSWDWRNVHGINFVSPVRNQASCGSCYSFASMGMLEARIRILTNNSQTPILSPQEVVSCSQYAQGCEGGFPYLIAGKYAQDFGLVEEACFPYTGTDSPCKMKEDCFRYYSSEYHYVGGFYGGCNEALMKLELVHHGPMAVAFEVYDDFLHYKKGIYHHTGLRDPFNPFELTNHAVLLVGYGTDSASGMDYWIVKNSWGTGWGENGYFRIRRGTDECAIESIAVAATPIPKL

SEQ ID NO: 35 - protein, 412 aa, Homo sapiens
MQPSSLLPLALCLLAAPASALVRIPLHKFTSIRRTMSEVGGSVEDLIAKGPVSKYSQAVPAVTEGPIPEVLKNYMDAQYYGEIGIGTPPQCFTVVFDTGSSNLWVPSIHCKLLDIACWIHHKYNSDKSSTYVKNGTSFDIHYGSGSLSGYLSQDTVSVPCQSASSASALGGVKVERQVFGEATKQPGITFIAAKFDGILGMAYPRISVNNVLPVFDNLMQQKLVDQNIFSFYLSRDPDAQPGGELMLGGTDSKYYKGSLSYLNVTRKAYWQVHLDQVEVASGLTLCKEGCEAIVDTGTSLMVGPVDEVRELQKAIGAVPLIQGEYMIPCEKVSTLPAITLKLGGKGYKLSPEDYTLKVSQAGKTLCLSGFMGMDIPPPSGPLWILGDVFIGRYYTVFDRDNNRVGFAEAARL

SEQ ID NO: 36 - protein, 401 aa, Homo sapiens
MKTLLLLLLVLLELGEAQGSLHRVPLRRHPSLKKKLRARSQLSEFWKSHNLDMIQFTESCSMDQSAKEPLINYLDMEYFGTISIGSPPQNFTVIFDTGSSNLWVPSVYCTSPACKTHSRFQPSQSSTYSQPGQSFSIQYGTGSLSGIIGADQVSAFATQVEGLTVVGQQFGESVTEPGQTFVDAEFDGILGLGYPSLAVGGVTPVFDNMMAQNLVDLPMFSVYMSSNPEGGAGSELIFGGYDHSHFSGSLNWVPVTKQAYWQIALDNIQVGGTVMFCSEGCQAIVDTGTSLITGPSDKIKQLQNAIGAAPVDGEYAVECANLNVMPDVTFTINGVPYTLSPTAYTLLDFVDGMQFCSSGFQGLDIHPPAGPLWILGDVFIRQFYSVFDRGNNRVGLAPAVP

SEQ ID NO: 37 - protein, 484 aa, Homo sapiens [sequence omitted]

SEQ ID NO: 38 - protein, 255 aa, Homo sapiens
MQPLLLLLAFLLPTGAEAGEIIGGRESRPHSRPYMAYLQIQSPAGQSRCGGFLVREDFVLTAAHCWGSNINVTLGAHNIQRRENTQQHITARRAIRHPQYNQRTIQNDIMLLQLSRRVRRNRNVNPVALPRAQEGLRPGTLCTVAGWGRVSMRRGTDTLREVQLRVQRDRQCLRIFGSYDPRRQICVGDRRERKAAFKGDSGGPLLCNNVAHGIVSYGKSSGVPPEVFTRVSSFLPWIRTTMRSFKLLDQMETPL

SEQ ID NO: 39 - protein, 335 aa, Homo sapiens
MWATLPLLCAGAWLLGVPVCGAAELCVNSLEKFHFKSWMSKHRKTYSTEEYHHRLQTFASNWRKINAHNNGNHTFKMALNQFSDMSFAEIKHKYLWSEPQNCSATKSNYLRGTGPYPPSVDWRKKGNFVSPVKNQGACGSCWTFSTTGALESAIAIATGKMLSLAEQQLVDCAQDFNNHGCQGGLPSQAFEYILYNKGIMGEDTYPYQGKDGYCKFQPGKAIGFVKDVANITIYDEEAMVEAVALYNPVSFAFEVTQDFMMYRTGIYSSTSCHKTPDKVNHAVLAVGYGEKNGIPYWIVKNSWGPQWGMNGYFLIERGKNMCGLAACASYPIPLV

SEQ ID NO: 40 - protein, 329 aa, Homo sapiens
MWGLKVLLLPVVSFALYPEEILDTHWELWKKTHRKQYNNKVDEISRRLIWEKNLKYISIHNLEASLGVHTYELAMNHLGDMTSEEVVQKMTGLKVPLSHSRSNDTLYIPEWEGRAPDSVDYRKKGYVTPVKNQGQCGSCWAFSSVGALEGQLKKKTGKLLNLSPQNLVDCVSENDGCGGGYMTNAFQYVQKNRGIDSEDAYPYVGQEESCMYNPTGKAAKCRGYREIPEGNEKALKRAVARVGPVSVAIDASLTSFQFYSKGVYYDESCNSDNLNHAVLAVGYGIQKGNKHWIIKNSWGENWGNKGYILMARNKNNACGIANLASFPKM

SEQ ID NO: 41 - protein, 333 aa, Homo sapiens [sequence omitted]

SEQ ID NO: 42 - protein, 334 aa, Homo sapiens
MNLSLVLAAFCLGIASAVPKFDQNLDTKWYQWKATHRRLYGANEEGWRRAVWEKNMKMIELHNGEYSQGKHGFTMAMNAFGDMTNEEFRQMMGCFRNQKFRKGKVFREPLFLDLPKSVDWRKKGYVTPVKNQKQCGSCWAFSATGALEGQMFRKTGKLVSLSEQNLVDCSRPQGNQGCNGGFMARAFQYVKENGGLDSEESYPYVAVDEICKYRPENSVANDTGFTVVAPGKEKALMKAVATVGPISVAMDAGHSSFQFYKSGIYFEPDCSSKNLDHGVLVVGYGFEGANSNNSKYWLVKNSWGPEWGSNGYVKIAKDKNNHCGIATAASYPNV

SEQ ID NO: 43 - protein, 321 aa, Homo sapiens
MDVRALPWLPWLLWLLCRGGGDADSRAPFTPTWPRSREREAAAFRESLNRHRYLNSLFPSENSTAFYGINQFSYLFPEEFKAIYLRSKPSKFPRYSAEVHMSIPNVSLPLRFDWRDKQVVTQVRNQQMCGGCWAFSVVGAVESAYAIKGKPLEDLSVQQVIDCSYNNYGCNGGSTLNALNWLNKMQVKLVKDSEYPFKAQNGLCHYFSGSHSGFSIKGYSAYDFSDQEDEMAKALLTFGPLVVIVDAVSWQDYLGGIIQHHCSSGEANHAVLITGFDKTGSTPYWIVRNSWGSSWGVDGYAHVKMGSNVCGIADSVSSIFV

SEQ ID NO: 44 - protein, 331 aa, Homo sapiens
MKRLVCVLLVCSSAVAQLHKDPTLDHHWHLWKKTYGKQYKEKNEEAVRRLIWEKNLKFVMLHNLEHSMGMHSYDLGMNHLGDMTSEEVMSLMSSLRVPSQWQRNITYKSNPNRILPDSVDWREKGCVTEVKYQGSCGACWAFSAVGALEAQLKLKTGKLVSLSAQNLVDCSTEKYGNKGCNGGFMTTAFQYIIDNKGIDSDASYPYKAMDQKCQYDSKYRAATCSKYTELPYGREDVLKEAVANKGPVSVGVDARHPSFFLYRSGVYYEPSCTQNVNHGVLVVGYGDLNGKEYWLVKNSWGHNFGEEGYIRMARNKGNHCGIASFPSYPEI

SEQ ID NO: 45 - protein, 376 aa, Homo sapiens
MALTAHPSCLLALLVAGLAQGIRGPLRAQDLGPQPLELKEAFKLFQIQFNRSYLSPEEHAHRLDIFAHNLAQAQRLQEEDLGTAEFGVTPFSDLTEEEFGQLYGYRRAAGGVPSMGREIRSEEPEESVPFSCDWRKVASAISPIKDQKNCNCCWAMAAAGNIETLWRISFWDFVDVSVQELLDCGRCGDGCHGGFVWDAFITVLNNSGLASEKDYPFQGKVRAHRCHPKKYQKVAWIQDFIMLQNNEHRIAQYLATYGPITVTINMKPLQLYRKGVIKATPTTCDPQLVDHSVLLVGFGSVKSEEGIWAETVSSQSQPQPPHPTPYWILKNSWGAQWGEKGYFRLHRGSNTCGITKFPLTARVQKPDMKPRVSCPP

SEQ ID NO: 46 - protein, 303 aa, Homo sapiens
MARRGPGWRPLLLLVLLAGAAQGGLYFRRGQTCYRPLRGDGLAPLGRSTYPRPHEYLSPADLPKSWDWRNVDGVNYASITRNQHIPQYCGSCWAHASTSAMADRINIKRKGAWPSTLLSVQNVIDCGNAGSCEGGNDLSVWDYAHQHGIPDETCNNYQAKDQECDKFNQCGTCNEFKECHAIRNYTLWRVGDYGSLSGREKMMAEIYANGPISCGIMATERLANYTGGIYAEYQDTTYINHVVSVAGWGISDGTEYWIVRNSWGEPWGERGWLRIVTSTYKDGKGARYNLAIEEHCTFGDPIV

SEQ ID NO: 47 - protein, 338 aa, Homo sapiens
MLQKPKSVKLRALRSPRKFGVAGRSCQEVLRKGCLRFQLPERGSRLCLYEDGTELTEDYFPSVPDNAELVLLTLGQAWQGYVSDIRRFLSAFHEPQVGLIQAAQQLLCDEQAPQRQRLLADLLHNVSQNIAAETRAEDPPWFEGLESRFQSKSGYLRYSCESRIRSYLREVSSYPSTVGAEAQEEFLRVLGSMCQRLRSMQYNGSYFDRGAKGGSRLCTPEGWFSCQGPFDMDSCLSRHSINPYSNRESRILFSTWNLDHIIEKKRTIIPTLVEAIKEQDGREVDWEYFYGLLFTSENLKLVHIVCHKKTTHKLNCDPSRIYKPQTRLKRKQPVRKRQ

SEQ ID NO: 48 - protein, 360 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 49 - protein, 364 aa, Homo sapiens
MSGPRAGFYRQELNKTVWEVPQRLQGLRPVGSGAYGSVCSAYDARLRQKVAVKKLSRPFQSLIHARRTYRELRLLKHLKHENVIGLLDVFTPATSIEDFSEVYLVTTLMGADLNNIVKCQALSDEHVQFLVYQLLRGLKYIHSAGIIHRDLKPSNVAVNEDCELRILDFGLARQADEEMTGYVATRWYRAPEIMLNWMHYNQTVDIWSVGCIMAELLQGKALFPGSDYIDQLKRIMEVVGTPSPEVLAKISSEHARTYIQSLPPMPQKDLSSIFRGANPLAIDLLGRMLVLDSDQRVSAAEALAHAYFSQYHDPEDEPEAEPYDESVEAKERTLEEWKELTYQEVLSFKPPEPPKPPGSLEIEQ

SEQ ID NO: 50 - protein, 367 aa, Homo sapiens
MSSPPPARSGFYRQEVTKTAWEVRAVYRDLQPVGSGAYGAVCSAVDGRTGAKVAIKKLYRPFQSELFAKRAYRELRLLKHMRHENVIGLLDVFTPDETLDDFTDFYLVMPFMGTDLGKLMKHEKLGEDRIQFLVYQMLKGLRYIHAAGIIHRDLKPGNLAVNEDCELKILDFGLARQADSEMTGYVVTRWYRAPEVILNWMRYTQTVDIWSVGCIMAEMITGKTLFKGSDHLDQLKEIMKVTGTPPAEFVQRLQSDEAKNYMKGLPELEKKDFASILTNASPLAVNLLEKMLVLDAEQRVTAGEALAHPYFESLHDTEDEPQVQKYDDSFDDVDRTLDEWKRVTYKEVLSFKPPRQLGARVSKETPL

SEQ ID NO: 51 - protein, 365 aa, Homo sapiens
MSLIRKKGFYKQDVNKTAWELPKTYVSPTHVGSGAYGSVCSAIDKRSGEKVAIKKLSRPFQSEIFAKRAYRELLLLKHMQHENVIGLLDVFTPASSLRNFYDFYLVMPFMQTDLQKIMGMEFSEEKIQYLVYQMLKGLKYIHSAGVVHRDLKPGNLAVNEDCELKILDFGLARHADAEMTGYVVTRWYRAPEVILSWMHYNQTVDIWSVGCIMAEMLTGKTLFKGKDYLDQLTQILKVTGVPGTEFVQKLNDKAAKSYIQSLPQTPRKDFTQLFPRASPQAADLLEKMLELDVDKRLTAAQALTHPFFEPFRDPEEETEAQQPFDDSLEHEKLTVDEWKQHIYKEIVNFSPIARKDSRRRSGMKL

SEQ ID NO: 52 - protein, 403 aa, Homo sapiens [sequence omitted]

SEQ ID NO: 53 - protein, 1154 aa, Homo sapiens
MQYLNIKEDCNAMAFCAKMRSSKKTEVNLEAPEPGVEVIFYLSDREPLRLGSGEYTAEELCIRAAQACRISPLCHNLFALYDENTKLWYAPNRTITVDDKMSLRLHYRMRFYFTNWHGTNDNEQSVWRHSPKKQKNGYEKKKIPDATPLLDASSLEYLFAQGQYDLVKCLAPIRDPKTEQDGHDIENECLGMAVLAISHYAMMKKMQLPELPKDISYKRYIPETLNKSIRQRNLLTRMRINNVFKDFLKEFNNKTICDSSVSTHDLKVKYLATLETLTKHYGAEIFETSMLLISSENEMNWFHSNDGGNVLYYEVMVTGNLGIQWRHKPNVVSVEKEKNKLKRKKLENKHKKDEEKNKIREEWNNFSYFPEITHIVIKESVVSINKQDNKKMELKLSSHEEALSFVSLVDGYFRLTADAHHYLCTDVAPPLIVHNIQNGCHGPICTEYAINKLRQEGSEEGMYVLRWSCTDFDNILMTVTCFEKSEQVQGAQKQFKNFQIEVQKGRYSLHGSDRSFPSLGDLMSHLKKQILRTDNISFMLKRCCQPKPREISNLLVATKKAQEWQPVYPMSQLSFDRILKKDLVQGEHLGRGTRTHIYSGTLMDYKDDEGTSEEKKIKVILKVLDPSHRDISLAFFEAASMMRQVSHKHIVYLYGVCVRDVENIMVEEFVEGGPLDLFMHRKSDVLTTPWKFKVAKQLASALSYLEDKDLVHGNVCTKNLLLAREGIDSECGPFIKLSDPGIPITVLSRQECIERIPWIAPECVEDSKNLSVAADKWSFGTTLWEICYNGEIPLKDKTLIEKERFYESRCRPVTPSCKELADLMTRCMNYDPNQRPFFRAIMRDINKLEEQNPDIVSEKKPATEVDPTHFEKRFLKRIRDLGEGHFGKVELCRYDPEGDNTGEQVAVKSLKPESGGNHIADLKKEIEILRNLYHENIVKYKGICTEDGGNGIKLIMEFLPSGSLKEYLPKNKNKINLKQQLKYAVQICKGMDYLGSRQYVHRDLAARNVLVESEHQVKIGDFGLTKAIETDKEYYTVKDDRDSPVFWYAPECLMQSKFYIASDVWSFGVTLHELLTYCDSDSSPMALFLKMIGPTHGQMTVTRLVNTLKEGKRLPCPPNCPDEVYQLMRKCWEFQPSNRTSFQNLIEGFEALLK

SEQ ID NO: 54 - protein, 1132 aa, Homo sapiens
MGMACLTMTEMEGTSTSSIYQNGDISGNANSMKQIDPVLQVYLYHSLGKSEADYLTFPSGEYVAEEICIAASKACGITPVYHNMFALMSETERIWYPPNHVFHIDESTRHNVLYRIRFYFPRWYCSGSNRAYRHGISRGAEAPLLDDFVMSYLFAQWRHDFVHGWIKVPVTHETQEECLGMAVLDMMRIAKENDQTPLAIYNSISYKTFLPKCIRAKIQDYHILTRKRIRYRFRRFIQQFSQCKATARNLKLKYLINLETLQSAFYTEKFEVKEPGSGPSGEEIFATIIITGNGGIQWSRGKHKESETLTEQDLQLYCDFPNIIDVSIKQANQEGSNESRVVTIHKQDGKNLEIELSSLREALSFVSLIDGYYRLTADAHHYLCKEVAPPAVLENIQSNCHGPISMDFAISKLKKAGNQTGLYVLRCSPKDFNKYFLTFAVERENVIEYKHCLITKNENEEYNLSGTKKNFSSLKDLLNCYQMETVRSDNIIFQFTKCCPPKPKDKSNLLVFRTNGVSDVPTSPTLQRPTHMNQMVFHKIRNEDLIFNESLGQGTFTKIFKGVRREVGDYGQLHETEVLLKVLDKAHRNYSESFFEAASMMSKLSHKHLVLNYGVCVCGDENILVQEFVKFGSLDTYLKKNKNCINILWKLEVAKQLAWAMHFLEENTLIHGNVCAKNILLIREEDRKTGNPPFIKLSDPGISITVLPKDILQERIPWVPPECIENPKNLNLATDKWSFGTTLWEICSGGDKPLSALDSQRKLQFYEDRHQLPAPKWAELANLINNCMDYEPDFRPSFRAIIRDLNSLFTPDYELLTENDMLPNMRIGALGFSGAFEDRDPTQFEERHLKFLQQLGKGNFGSVEMCRYDPLQDNTGEVVAVKKLQHSTEEHLRDFEREIEILKSLQHDNIVKYKGVCYSAGRRNLKLIMEYLPYGSLRDYLQKHKERIDHIKLLQYTSQICKGMEYLGTKRYIHRDLATRNILVENENRVKIGDFGLTKVLPQDKEYYKVKEPGESPIFWYAPESLTESKFSVASDVWSFGVVLYELFTYIEKSKSPPAEFMRMIGNDKQGQMIVFHLIELLKNNGRLPRPDGCPDEIYMIMTECWNNNVNQRPSFRDLALRVDQIRDNMAG

SEQ ID NO: 55 - protein, 1124 aa, Homo sapiens
MAPPSEETPLIPQRSCSLLSTEAGALHVLLPARGPGPPQRLSFSFGDHLAEDLCVQAAKASGILPVYHSLFALATEDLSCWFPPSHIFSVEDASTQVLLYRIRFYFPNWFGLEKCHRFGLRKDLASAILDLPVLEHLFAQHRSDLVSGRLPVGLSLKEQGECLSLAVLDLARMAREQAQRPGELLKTVSYKACLPPSLRDLIQGLSFVTRRRIRRTVRRALRRVAACQADRHSLMAKYIMDLERLDPAGAAETFHVGLPGALGGHDGLGLLRVAGDGGIAWTQGEQEVLQPFCDFPEIVDISIKQAPRVGPAGEHRLVTVTRTDNQILEAEFPGLPEALSFVALVDGYFRLTTDSQHFFCKEVAPPRLLEEVAEQCHGPITLDFAINKLKTGGSRPGSYVLRRSPQDFDSFLLTVCVQNPLGPDYKGCLIRRSPTGTFLLVGLSRPHSSLRELLATCWDGGLHVDGVAVTLTSCCIPRPKEKSNLIVVQRGHSPPTSSLVQPQSQYQLSQMTFHKIPADSLEWHENLGHGSFTKIYRGCRHEVVDGEARKTEVLLKVMDAKHKNCMESFLEAASLMSQVSYRHLVLLHGVCMAGDSTMVQEFVHLGAIDMYLRKRGHLVPASWKLQVVKQLAYALNYLEDKGLPHGNVSARKVLLAREGADGSPPFIKLSDPGVSPAVLSLEMLTDRIPWVAPECLREAQTLSLEADKWGFGATVWEVFSGVTMPISALDPAKKLQFYEDRQQLPAPKWTELALLIQQCMAYEPVQRPSFRAVIRDLNSLISSDYELLSDPTPGALAPRDGLWNGAQLYACQDPTIFEERHLKYISQLGKGNFGSVELCRYDPLGDNTGALVAVKQLQHSGPDQQRDFQREIQILKALHSDFIVKYRGVSYGPGRQSLRLVMEYLPSGCLRDFLQRHRARLDASRLLLYSSQICKGMEYLGSRRCVHRDLAARNILVESEAHVKIADFGLAKLLPLDKDYYVVREPGQSPIFWYAPESLSDNIFSRQSDVWSFGVVLYELFTYCDKSCSPSAEFLRMMGCERDVPALCRLLELLEEGQRLPAPPACPAEVHELMKLCWAPSPQDRPSFSALGPQLDMLWSGSRGCETHAFTAHPEGKHHSLSFS

SEQ ID NO: 56 - protein, 1187 aa, Homo sapiens
MPLRHWGMARGSKPVGDGAQPMAAMGGLKVLLHWAGPGGGEPWVTFSESSLTAEEVCIHIAHKVGITPPCFNLFALFDAQAQVWLPPNHILEIPRDASLMLYFRIRFYFRNWHGMNPREPAVYRCGPPGTEASSDQTAQGMQLLDPASFEYLFEQGKHEFVNDVASLWELSTEEEIHHFKNESLGMAFLHLCHLALRHGIPLEEVAKKTSFKDCIPRSFRRHIRQHSALTRLRLRNVFRRFLRDFQPGRLSQQMVMVKYLATLERLAPRFGTERVPVCHLRLLAQAEGEPCYIRDSGVAPTDPGPESAAGPPTHEVLVTGTGGIQWWPVEEEVNKEEGSSGSSGRNPQASLFGKKAKAHKAVGQPADRPREPLWAYFCDFRDITHVVLKEHCVSIHRQDNKCLELSLPSRAAALSFVSLVDGYFRLTADSSHYLCHEVAPPRLVMSIRDGIHGPLLEPFVQAKLRPEDGLYLIHWSTSHPYRLILTVAQRSQAPDGMQSLRLRKFPIEQQDGAFVLEGWGRSFPSVRELGAALQGCLLRAGDDCFSLRRCCLPQPGETSNLIIMRGARASPRTLNLSQLSFHRVDQKEITQLSHLGQGTRTNVYEGRLRVEGSGDPEEGKMDDEDPLVPGRDRGQELRVVLKVLDPSHHDIALAFYETASLMSQVSHTHLAFVHGVCVRGPENIMVTEYVEHGPLDVWLRRERGHVPMAWKMVVAQQLASALSYLENKNLVHGNVCGRNILLARLGLAEGTSPFIKLSDPGVGLGALSREERVERIPWLAPECLPGGANSLSTAMDKWGFGATLLEICFDGEAPLQSRSPSEKEHFYQRQHRLPEPSCPQLATLTSQCLTYEPTQRPSFRTILRDLTRLQPHNLADVLTVNPDSPASDPTVFHKRYLKKIRDLGEGHFGKVSLYCYDPTNDGTGEMVAVKALKADCGPQHRSGWKQEIDILRTLYHEHIIKYKGCCEDQGEKSLQLVMEYVPLGSLRDYLPRHSIGLAQLLLFAQQICEGMAYLHAQHYIHRDLAARNVLLDNDRLVKIGDFGLAKAVPEGHEYYRVREDGDSPVFWYAPECLKEYKFYYASDVWSFGVTLYELLTHCDSSQSPPTKFLELIGIAQGQMTVLRLTELLERGERLPRPDKCPCEVYHLMKNCWETEASFRPTFENLIPILKTVHEKYQGQAPSVFSVC

SEQ ID NO: 57 - protein, 906 aa, Homo sapiens
MAGNVKKSSGAGGGSGSGGSGSGGLIGLMKDAFQPHHHHHHHLSPHPPGTVDKKMVEKCWKLMDKVVRLCQNPKLALKNSPPYILDLLPDTYQHLRTILSRYEGKMETLGENEYFRVFMENLMKKTKQTISLFKEGKERMYEENSQPRRNLTKLSLIFSHMLAELKGIFPSGLFQGDTFRITKADAAEFWRKAFGEKTIVPWKSFRQALHEVHPISSGLEAMALKSTIDLTCNDYISVFEFDIFTRLFQPWSSLLRNWNSLAVTHPGYMAFLTYDEVKARLQKFIHKPGSYIFRLSCTRLGQWAIGYVTADGNILQTIPHNKPLFQALIDGFREGFYLFPDGRNQNPDLTGLCEPTPQDHIKVTQEQYELYCEMGSTFQLCKICAENDKDVKIEPCGHLMCTSCLTSWQESEGQGCPFCRCEIKGTEPIVVDPFDPRGSGSLLRQGAEGAPSPNYDDDDDERADDTLFMMKELAGAKVERPPSPFSMAPQASLPPVPPRLDLLPQRVCVPSSASALGTASKAASGSLHKDKPLPVPPTLRDLPPPPPPDRPYSVGAESRPQRRPLPCTPGDCPSRDKLPPVPSSRLGDSWLPRPIPKVPVSAPSSSDPWTGRELTNRHSLPFSLPSQMEPRPDVPRLGSTFSLDTSMSMNSSPLVGPECDHPKIKPSSSANAIYSLAARPLPVPKLPPGEQCEGEEDTEYMTPSSRPLRPLDTSQSSRACDCDQQIDSCTYEAMYNIQSQAPSITESSTFGEGNLAAAHANTGPEESENEDDGYDVPKPPVPAVLARRTLSDISNASSSFGWLSLDGDPTTNVTEGSQVPERPPKPFPRRINSERKAGSCQQGSGPAASAATASPQLSSEIENLMSQGYSYQDIQKALVIAQNNIEMAKNILREFVSISSPAHVAT

SEQ ID NO: 58 - protein, 465 aa, Homo sapiens
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 59 - protein, 108 aa, Homo sapiens [sequence omitted]

SEQ ID NO: 60 - protein, 501 aa, Homo sapiens
MAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEACSRQHRLDQDKIEALSSKVQQLERSIGLKDLAMADLEQKVLEMEASTYDGVFIWKISDFARKRQEAVAGRIPAIFSPAFYTSRYGYKMCLRIYLNGDGTGRGTHLSLFFVVMKGPNDALLRWPFNQKVTLMLLDQNNREHVIDAFRPDVTSSSFQRPVNDMNIASGCPLFCPVSKMEAKNSYVRDDAIFIKAIVDLTGL

SEQ ID NO: 61 - protein, 491 aa, Homo sapiens
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 62 - protein, 240 aa, Renilla reniformis
MTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNY

SEQ ID NO: 63 - protein, 320 aa, Homo sapiens
MELLSPPLRDVDLTAPDGSLCSFATTDDFYDDPCFDSPDLRFFEDLDPRLMHVGALLKPEEHSHFPAAVHPAPGAREDEHVRAPSGHHQAGRCLLWACKACKRKTTNADRRKAATMRERRRLSKVNEAFETLKRCTSSNPNQRLPKVEILRNAIRYIEGLQALLRDQDAAPPGAAAAFYAPGPLPPGRGGEHYSGDSDASSPRSNCSDGMMDYSGPPSGARRRNCYEGAYYNEAPSEPRPGKSAAVSSLDCLSSIVERISTESPAAPALLLADVPSESPPRRQEAAAPSEGESSGDPTQSPDAAPQCPAGANPNPIYQVL

SEQ ID NO: 64 - protein, 607 aa, Homo sapiens
MAEKRRGSPCSMLSLKAHAFSVEALIGAEKQQQLQKKRRKLGAEEAAGAVDDGGCSRGGGAGEKGSSEGDEGAALPPPAGATSGPARSGADLERGAAGGCEDGFQQGASPLASPGGSPKGSPARSLARPGTPLPSPQAPRVDLQGAELWKRFHEIGTEMIITKAGRRMFPAMRVKISGLDPHQQYYIAMDIVPVDNKRYRYVYHSSKWMVAGNADSPVPPRVYIHPDSPASGETWMRQVISFDKLKLTNNELDDQGHIILHSMHKYQPRVHVIRKDCGDDLSPIKPVPSGEGVKAFSFPETVFTTVTAYQNQQITRLKIDRNPFAKGFRDSGRNRMGLEALVESYAFWRPSLRTLTFEDIPGIPKQGNASSSTLLQGTGNGVPATHPHLLSGSSCSSPAFHLGPNTSQLCSLAPADYSACARSGLTLNRYSTSLAETYNRLTNQAGETFAPPRTPSYVGVSSSTSVNMSMGGTDGDTFSCPQTSLSMQISGMSPQLQYIMPSPSSNAFATNQTHQGSYNTFRLHSPCALYGYNFSTSPKLAASPEKIVSSQGSFLGSSPSGTMTDRQMLPPVEGVHLLSSGGQQSFFDSRTLGSLTLSSSQVSAHMV

SEQ ID NO: 65 - protein, 393 aa, Homo sapiens [sequence omitted]

SEQ ID NO: 66 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 67 - protein, 356 aa, Homo sapiens
MTKSYSESGLMGEPQPQGPPSWTDECLSSQDEEHEADKKEDDLETMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 68 - protein, 498 aa, Homo sapiens
MNQSIPVAPTPPRRVRLKPWLVAQVNSCQYPGLQWVNGEKKLFCIPWRHATRHGPSQDGDNTIFKAWAKETGKYTEGVDEADPAKWKANLRCALNKSRDFRLIYDGPRDMPPQPYKIYEVCSNGPAPTDSQPPEDYSFGAGEEEEEEEELQRMLPSLSLTEDVKWPPTLQPPTLRPPTLQPPTLQPPVVLGPPAPDPSPLAPPPGNPAGFRELLSEVLEPGPLPASLPPAGEQLLPDLLISPHMLPLTDLEIKFQYRGRPPRALTISNPHGCRLFYSQLEATQEQVELFGPISLEQVRFPSPEDIPSDKQRFYTNQLLDVLDRGLILQLQGQDLYAIRLCQCKVFWSGPCASAHDSCPNPIQREVKTKLFSLEHFLNELILFQKGQTNTPPPFEIFFCFGEEWPDRKPREKKLITVQVVPVAARLLLEMFSGELSWSADSIRLQISNPDLKDRMVEQFKELHHIWQSQQRLQPVAQAPPGAGLGVGQGPWPMHPAGMQ

SEQ ID NO: 69 - protein, 427 aa, Homo sapiens
MGTPKPRILPWLVSQLDLGQLEGVAWVNKSRTRFRIPWKHGLRQDAQQEDFGIFQAWAEATGAYVPGRDKPDLPTWKRNFRSALNRKEGLRLAEDRSKDPHDPHKIYEFVNSGVGDFSQPDTSPDTNGGGSTSDTQEDILDELLGNMVLAPLPDPGPPSLAVAPEPCPQPLRSPSLDNPTPFPNLGPSENPLKRLLVPGEEWEFEVTAFYRGRQVFQQTISCPEGLRLVGSEVGDRTLPGWPVTLPDPGMSLTDRGVMSYVRHVLSCLGGGLALWRAGQWLWAQRLGHCHTYWAVSEELLPNSGHGPDGEVPKDKEGGVFDLGPFIVDLITFTEGSGRSPRYALWFCVGESWPQDQPWTKRLVMVKVVPTCLRALVEMARVGGASSLENTVDLHISNSHPLSLTSDQYKAYLQDLVEGMDFQGPGES

SEQ ID NO: 70 - protein, 750 aa, Homo sapiens
MSQWYELQQLDSKFLEQVHQLYDDSFPMEIRQYLAQWLEKQDWEHAANDVSFATIRFHDLLSQLDDQYSRFSLENNFLLQHNIRKSKRNLQDNFQEDPIQMSMIIYSCLKEERKILENAQRFNQAQSGNIQSTVMLDKQKELDSKVRNVKDKVMCIEHEIKSLEDLQDEYDFKCKTLQNREHETNGVAKSDQKQEQLLLKKMYLMLDNKRKEVVHKIIELLNVTELTQNALINDELVEWKRRQQSACIGGPPNACLDQLQNWFTIVAESLQQVRQQLKKLEELEQKYTYEHDPITKNKQVLWDRTFSLFQQLIQSSFVVERQPCMPTHPQRPLVLKTGVQFTVKLRLLVKLQELNYNLKVKVLFDKDVNERNTVKGFRKFNILGTHTKVMNMEESTNGSLAAEFRHLQLKEQKNAGTRTNEGPLIVTEELHSLSFETQLCQPGLVIDLETTSLPVVVISNVSQLPSGWASILWYNMLVAEPRNLSFFLTPPCARWAQLSEVLSWQFSSVTKRGLNVDQLNMLGEKLLGPNASPDGLIPWTRFCKENINDKNFPFWLWIESILELIKKHLLPLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDGPKGTGYIKTELISVSEVHPSRLQTTDNLLPMSPEEFDEVSRIVGSVEFDSMMNTV

SEQ ID NO: 71 - protein, 225 aa, Homo sapiens
MVTHSKFPAAGMSRPLDTSLRLKTFSSKSEYQLVVNAVRKLQESGFYWSAVTGGEANLLLSAEPAGTFLIRDSSDQRHFFTLSVKTQSGTKNLRIQCEGGSFSLQSDPRSTQPVPRFDCVLKLVHHYMPPPGAPSFPSPPTEPSSEVPEQPSAQPLPGSPPRRAYYIYSGGEKIPLVLSRPLSSNVATLQHLCRKTVNGHLDSYEKVTQLPGPIREFLDQYDAPL

SEQ ID NO: 72 - protein, 770 aa, Homo sapiens
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 73 - protein, 246 aa, Homo sapiens
MELVLTQTPSPVSAVVGGTVTINCQSSQSVWGNNRLSWYQQKPGQPPRLLMYYASNLASGVSSRFKGSGSGTQFTLTISDVQCDDAATYYCQGGFECSGGDCVGFGGGTELEILGGSSRSSSSGGGGSGGGGQSVEESGGRLVAPGGSLTLTCTVSGIDLSSDAMSWVRQAPGKGLEWIGTIYGSAGTYYATWAKGRFTISKTSTTVDLKMTSLTTEDTATYFCTRAFSNTRLDLWGQGTLVTISS

SEQ ID NO: 74 - protein, 233 aa, Homo sapiens
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 75 - protein, 312 aa, Homo sapiens
MANNDAVLKRLEQKGAEADQIIEYLKQQVSLLKEKAILQATLREEKKLRVENAKLKKEIEELKQELIQAEIQNGVKQIPFPSGTPLHANSMVSENVIQSTAVTTVSSGTKEQIKGGTGDEKKAKEKIEKKGEKKEKKQQSIAGSADSKPIDVSRLDLRIGCIITARKHPDADSLYVEEVDVGEIAPRTVVSGLVNHVPLEQMQNRMVILLCNLKPAKMRGVLSQAMVMCASSPEKIEILAPPNGSVPGDRITFDAFPGEPDKELNPKKKIWEQIQPDLHTNDECVATYKGVPFEVKGKGVCRAQTMSNSGIK

SEQ ID NO: 76 - protein, 320 aa, Homo sapiens
MPMYQVKPYHGGGAPLRVELPTCMYRLPNVHGRSYGPAPGAGHVQEESNLSLQALESRQDDILKRLYELKAAVDGLSKMIQTPDADLDVTNIIQADEPTTLTTNALDLNSVLGKDYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPFNTALKLLK

SEQ ID NO: 77 - protein, 233 aa, Homo sapiens
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDE

SEQ ID NO: 78 - protein, 267 aa, Homo sapiens
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSGSGLRSRAQASNSAVDGTAGPGSTGS

SEQ ID NO: 79 - protein, 425 aa, Homo sapiens
MADKEAAFDDAVEERVINEEYKIWKKNTPFLYDLVMTHALEWPSLTAQWLPDVTRPEGKDFSIHRLVLGTHTSDEQNHLVIASVQLPNDDAQFDASHYDSEKGEFGGFGSVSGKIEIEIKINHEGEVNRARYMPQNPCIIATKTPSSDVLVFDYTKHPSKPDPSGECNPDLRLRGHQKEGYGLSWNPNLSGHLLSASDDHTICLWDISAVPKEGKVVDAKTIFTGHTAVVEDVSWHLLHESLFGSVADDQKLMIWDTRSNNTSKPSHSVDAHTAEVNCLSFNPYSEFILATGSADKTVALWDLRNLKLKLHSFESHKDEIFQVQWSPHNETILASSGTDRRLNVWDLSKIGEEQSPEDAEDGPPELLFIHGGHTAKISDFSWNPNEPWVICSVSEDNIMQVWQMAENIYNDEDPEGSVDPEGQGS

SEQ ID NO: 80 - protein, 375 aa, Homo sapiens
MADHSFSDGVPSDSVEAAKNASNTEKLTDQVMQNPRVLAALQERLDNVPHTPSSYIETLPKAVKRRINALKQLQVRCAHIEAKFYEEVHDLERKYAALYQPLFDKRREFITGDVEPTDAESEWHSENEEEEKLAGDMKSKVVVTEKAAATAEEPDPKGIPEFWFTIFRNVDMLSELVQEYDEPILKHLQDIKVKFSDPGQPMSFVLEFHFEPNDYFTNSVLTKTYKMKSEPDKADPFSFEGPEIVDCDGCTIDWKKGKNVTVKTIKKKQKHKGRGTVRTITKQVPNESFFNFFNPLKASGDGESLDEDSEFTLASDFEIGHFFRERIVPRAVLYFTGEAIEDDDNFEEGEEGEEEELEGDEEGEDEDDAEINPKV

SEQ ID NO: 81 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 82 - protein, 462 aa, Homo sapiens
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHESGASIKIDEPLEGSEDRIITITGTQDQIQNAQYLLQNSVKQYSGKF

SEQ ID NO: 83 - protein, 745 aa, Homo sapiens
MERPPGLRPGAGGPWEMRERLGTGGFGNVCLYQHRELDLKIAIKSCRLELSTKNRERWCHEIQIMKKLNHANVVKACDVPEELNILIHDVPLLAMEYCSGGDLRKLLNKPENCCGLKESQILSLLSDIGSGIRYLHENKIIHRDLKPENIVLQDVGGKIIHKIIDLGYAKDVDQGSLCTSFVGTLQYLAPELFENKPYTATVDYWSFGTMVFECIAGYRPFLHHLQPFTWHEKIKKKDPKCIFACEEMSGEVRFSSHLPQPNSLCSLVVEPMENWLQLMLNWDPQQRGGPVDLTLKQPRCFVLMDHILNLKIVHILNMTSAKIISFLLPPDESLHSLQSRIERETGINTGSQELLSETGISLDPRKPASQCVLDGVRGCDSYMVYLFDKSKTVYEGPFASRSLSDCVNYIVQDSKIQLPIIQLRKVWAEAVHYVSGLKEDYSRLFQGQRAAMLSLLRYNANLTKMKNTLISASQQLKAKLEFFHKSIQLDLERYSEQMTYGISSEKMLKAWKEMEEKAIHYAEVGVIGYLEDQIMSLHAEIMELQKSPYGRRQGDLMESLEQRAIDLYKQLKHRPSDHSYSDSTEMVKIIVHTVQSQDRVLKELFGHLSKLLGCKQKIIDLLPKVEVALSNIKEADNTVMFMQGKRQKEIWHLLKIACTQSSARSLVGSSLEGAVTPQTSAWLPPTSAEHDHSLSCVVTPQDGETSAQMIEENLNCLGHLSTIIHEANEEQGNSMMNLDWSWLTE

SEQ ID NO: 84 - protein, 968 aa, Homo sapiens
MAEDDPYLGRPEQMFHLDPSLTHTIFNPEVFQPQMALPTDGPYLQILEQPKQRGFRFRYVCEGPSHGGLPGASSEKNKKSYPQVKICNYVGPAKVIVQLVTNGKNIHLHAHSLVGKHCEDGICTVTAGPKDMVVGFANLGILHVTKKKVFETLEARMTEACIRGYNPGLLVHPDLAYLQAEGGGDRQLGDREKELIRQAALQQTKEMDLSVVRLMFTAFLPDSTGSFTRRLEPVVSDAIYDSKAPNASNLKIVRMDRTAGCVTGGEEIYLLCDKVQKDDIQIRFYEEEENGGVWEGFGDFSPTDVHRQFAIVFKTPKYKDINITKPASVFVQLRRKSDLETSEPKPFLYYPEIKDKEEVQRKRQKLMPNFSDSFGGGSGAGAGGGGMFGSGGGGGGTGSTGPGYSFPHYGFPTYGGITFHPGTTKSNAGMKHGTMDTESKKDPEGCDKSDDKNTVNLFGKVIETTEQDQEPSEATVGNGEVTLTYATGTKEESAGVQDNLFLEKAMQLAKRHANALFDYAVTGDVKMLLAVQRHLTAVQDENGDSVLHLAIIHLHSQLVRDLLEVTSGLISDDIINMRNDLYQTPLHLAVITKQEDVVEDLLRAGADLSLLDRLGNSVLHLAAKEGHDKVLSILLKHKKAALLLDHPNGDGLNAIHLAMMSNSLPCLLLLVAAGADVNAQEQKSGRTALHLAVEHDNISLAGCLLLEGDAHVDSTTYDGTTPLHIAAGRGSTRLAALLKAAGADPLVENFEPLYDLDDSWENAGEDEGVVPGTTPLDMATSWQVFDILNGKPYEPEFTSDDLLAQGDMKQLAEDVKLQLYKLLEIPDPDKNWATLAQKLGLGILNNAFRLSPAPSKTLMDNYEVSGGTVRELVEALRQMGYTEAIEVIQAASSPVKTTSQAHSLPLSPASTRQQIDELRDSDSVCDSGVETSFRKLSFTESLTSGASLLTLNKMPHDYGQEGPLEGKI